(19) United States
(12) Patent Application Publication
(10) Pub. No.: US 2023/0218563 A1
(43) Pub. Date: Jul. 13, 2023

(54) METHODS FOR TREATING OR PREVENTING CHRONIC KIDNEY DISEASE

(71) Applicant: Arthrosi Therapeutics, Inc., Laguna Hills, CA (US)

(72) Inventors: Litain YEH, San Diego, CA (US); Shunqi YAN, San Diego, CA (US); Rongzi YAN, San Diego, CA (US); Zancong SHEN, San Diego, CA (US)

(21) Appl. No.: 18/001,427

(22) PCT Filed: Jun. 9, 2021
(86) PCT No.: PCT/US2021/036620

§ 371 Date: Dec. 9, 2022

Related U.S. Application Data
(60) Provisional application No. 63/195,411, filed on Jun. 1, 2021.
(60) Provisional application No. 63/037,469, filed on Jun. 10, 2020.

(51) Int. Cl. A61K 31/343; A61K 31/519; A61K 31/381; A61K 31/7048; A61P 13/12; A61P 9/04

(52) U.S. Cl. CPC A61K 31/343; A61K 31/519; A61K 31/381; A61K 31/7048; A61P 13/12; A61P 9/04

(57) ABSTRACT

Described herein are methods for treating or preventing chronic kidney disease, and other diseases, comprising the administration (3,5-dibromo-4-hydroxyphenyl)(2-(1-hydroxyethy)benzofuran-3-yl-4,5,6,7-d₄)methanone, and solvates thereof.

### CROSS-REFERENCE

This application claims benefit of U.S. Provisional Patent Application No. 63/037,469, filed on Jun. 10, 2020, and U.S. Provisional Patent Application No. 63/195,411, filed on Jun. 1, 2021, both of which are incorporated herein by reference in their entirety.

### BACKGROUND

Chronic kidney disease describes the gradual loss of kidney function. Kidneys filter wastes and excess fluids from the blood, which are then excreted in the urine. When chronic kidney disease reaches an advanced stage, dangerous levels of fluid, electrolytes, and wastes can build up in the body. Chronic kidney disease can progress to end-stage kidney failure, which is fatal without dialysis or a kidney transplant. New chronic kidney disease medications are needed to treat or prevent this disease.

### SUMMARY OF THE INVENTION

In one aspect, described herein is a method for treating or preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone, or solvate thereof. In some embodiments is a method for treating chronic kidney disease in an individual in need thereof. In some embodiments is a method for preventing chronic kidney disease in an individual in need thereof.

In another aspect, described herein is a method for treating or preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone, or solvate thereof. In some embodiments is a method for treating heart failure in an individual in need thereof. In some embodiments is a method for preventing heart failure in an individual in need thereof.

In another aspect, described herein is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, comprising administering to the individual a 3-yl-4,5,6,7-d₄)methanone, or solvate thereof. In some embodiments is a method for treating heart failure in an individual in need thereof. In some embodiments is a method for preventing heart failure in an individual in need thereof.

In some embodiments, the therapeutically effective amount of (3,5-dibromo hydroxyphenyl)(2-(1-hydroxyethyl)benzofuran-3-yl-4,5,6,7-d₄)methanone, or solvate thereof, is from about 3 mg to about 1500 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 3 mg to about 600 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 5 mg to about 300 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 10 mg to about 200 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 10 mg to about 100 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered orally. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is taken with food. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is taken without food. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered to the individual once per day. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered to the individual twice per day. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with at least one additional therapeutic agent. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor selected from allopurinol, oxypurinol, febuxostat, topiroxostat, and inositol. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with a sodium-glucose co-transporter-2 (SGLT2) inhibitor. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with an SGLT2 inhibitor selected from canagliflozin, dapagliflozin, empagliflozin, empagliflozin/linagliptin, empagliflozin/metformin, and dapagliflozin/metformin. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor and a SGLT2 inhibitor. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor and a SGLT2 inhibitor, wherein the xanthine oxidase inhibitor is selected from allopurinol, oxypurinol, febuxostat, topiroxostat, and inositol, and the SGLT2 inhibitor is selected from canagliflozin, dapagliflozin, empagliflozin, empagliflozin/linagliptin, empagliflozin/metformin, and dapagliflozin/metformin.

In another aspect is a pharmaceutical composition for use in the treatment or prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone and at least one inactive ingredient selected from pharmaceutically acceptable carriers, diluents, and excipients. In some embodiments is a pharmaceutical composition for use in the treatment of chronic kidney disease in an individual in need thereof. In some embodiments is a pharmaceutical composition for use in the prevention of chronic kidney disease in an individual in need thereof.

In another aspect is a pharmaceutical composition for use in the treatment or prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone and at least one inactive ingredient selected from pharmaceutically acceptable carriers, diluents, and excipients. In some embodiments is a pharmaceutical composition for use in the treatment of heart failure in an individual in need thereof. In some embodiments is a pharmaceutical composition for use in the prevention of heart failure in an individual in need thereof.

In another aspect is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a 3-yl-4,5,6,7-d₄)methanone and at least one inactive ingredient selected from pharmaceutically acceptable carriers, diluents, and excipients.

In some embodiments, the therapeutically effective amount of (3,5-dibromo hydroxyphenyl)(2-(1-hydroxyethyl)benzofuran-3-yl-4,5,6,7-d₄)methanone, or solvate thereof, is from about 3 mg to about 1500 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 3 mg to about 600 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 5 mg to about 300 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 10 mg to about 200 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 10 mg to about 100 mg. In some embodiments, the therapeutically effective amount of (3,5-dibromo-4-hydroxyphenyl)(2-(1-hydroxyethyl)benzofuran-3-yl-4,5,6,7-d₄)methanone, or solvate thereof, is from about 25 mg to about 75 mg. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered orally. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is taken with food. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is taken without food. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered to the individual once per day. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered to the individual twice per day. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered with at least one additional therapeutic agent. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor. In some embodiments, the pharmaceutical composition of (3,5-dibromo-4-hydroxyphenyl)(2-(1-hydroxyethyl)benzofuran yl-4,5,6,7-d₄)methanone, or solvate thereof, is administered with a xanthine oxidase inhibitor selected from allopurinol, oxypurinol, febuxostat, topiroxostat, and inositol. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered with an SGLT2 inhibitor. In some embodiments, the pharmaceutical composition of (3,5-dibromo hydroxyphenyl)(2-(1-hydroxyethyl)benzofuran-3-yl-4,5,6,7-d₄)methanone, or solvate thereof, is administered with an SGLT2 inhibitor selected from canagliflozin, dapagliflozin, empagliflozin, empagliflozin/linagliptin, empagliflozin/metformin, and dapagliflozin/metformin. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor and an SGLT2 inhibitor. In some embodiments, the pharmaceutical composition of (3,5-dibromo-4-hydroxyphenyl)(2-(1-hydroxyethyl)benzofuran-3-yl-4,5,6,7-d₄)methanone, or solvate thereof, is administered with a xanthine oxidase inhibitor and an SGLT2 inhibitor, wherein the xanthine oxidase inhibitor is selected from allopurinol, oxypurinol, febuxostat, topiroxostat, and inositol, and the SGLT2 inhibitor is selected from canagliflozin, dapagliflozin, empagliflozin, empagliflozin/linagliptin, empagliflozin/metformin, and dapagliflozin/metformin.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1. Illustrates the mean plasma concentration profiles of Compound 1 following a single oral dose of Compound 1 (15 mg, 50 mg, 100 mg, and 150 mg) in the fasted state.

FIG. 2. Illustrates the mean plasma concentration profiles of Compound 1 following a single oral dose of Compound 1 at 50 mg in the fasted versus fed state.

FIG. 3. Illustrates the dose proportionality of Compound 1 AUC following a single oral dose of Compound 1 (15 mg, 50 mg, 100 mg, and 150 mg) in the fasted state.

FIG. 4. Illustrates the dose proportionality of Compound 1 Cmax following a single oral dose of Compound 1 (15 mg, 50 mg, 100 mg, and 150 mg) in the fasted state.

FIG. 5. Illustrates mean serum uric acid levels (mg/dL) following a single oral dose of Compound 1 at various doses under fasted conditions.

FIG. 6. Illustrates mean time-matched (Day-1) percent change in serum uric acid concentration from baseline following a single oral dose of Compound 1 at various doses under fasted conditions.

FIG. 7. Illustrates mean time-matched (Day-1) percent change in serum uric acid concentration from baseline following a single oral dose of Compound 1 at 50 mg in the fasted versus fed state.

FIG. 8. Illustrates the mean plasma concentration profiles of Compound 1 following once-daily oral doses of Compound 1 for 10 days at various doses under fasted conditions.

FIG. 9. Illustrates mean serum uric acid levels (mg/dL) following once-daily oral doses of Compound 1 for 10 days at various doses under fasted conditions.

FIG. 10. Illustrates mean time-matched (Day-1) percent change in serum uric acid concentration from baseline following once-daily oral doses of Compound 1 for 10 days at various doses under fasted conditions.

### DETAILED DESCRIPTION OF THE INVENTION

Benzbromarone is a uricosuric agent effective in lowering serum uric acid (sUA). It has been found that therapy using benzbromarone can lead to lowering of sUA even following a single dose and continue to be lowered following multiple doses, and that chronic therapy can bring sUA into target levels of <6 mg/dL. However, in certain patients, benzbromarone is associated with hepatotoxicity. A high proportion of these patients developed acute liver failure leading to death or emergency liver transplantation. As a result, benzbromarone was never approved for use in the United States. In addition, the hepatotoxicity of benzbromarone led to its withdrawal in Europe in 2003. Benzbromarone is converted to reactive metabolites by CYP2C9. Benzbromarone is metabolized to 5,6-dihydroxybenzbromarone via 6-OH benzbromarone by CYP2C9, followed by the oxidation of 5,6-dihydroxybenzbromarone to a reactive ortho-quinone intermediate. The mechanism of benzbromarone hepatotoxicity is believed to be a result of its hepatic metabolism by CYP2C9 and possible effects of the 6-OH benzbromarone and its further metabolites on mitochondrial function (Iwamura et al., Drug Metabolism and Disposition, 2011, 39, 838-846; Uchida et al., Drug Metab. Pharmacokinet., 2010, 25, 605-610).

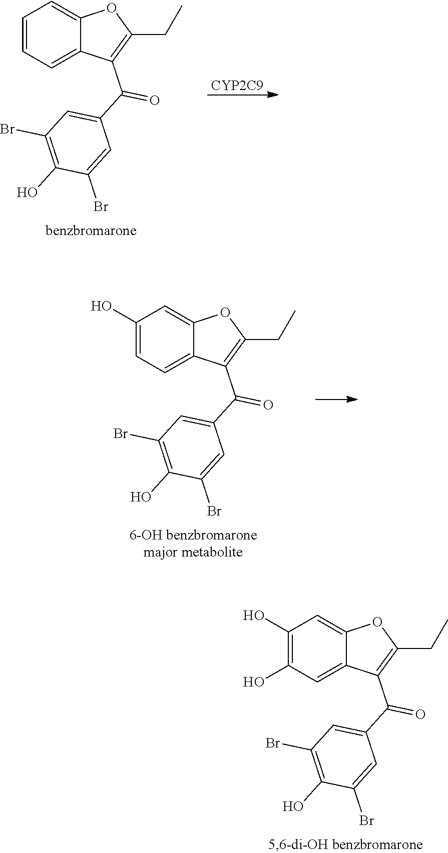

Described herein is (3,5-dibromo-4-hydroxyphenyl)(2-(1-hydroxyethyl)benzofuran-3-yl-4,5,6,7-d₄)methanone (Compound 1), a 4,5,6,7-tertradeutero analog of benzbromarone. Compound 1 showed better in vitro URAT1 potency than benzbromarone. Compound 1 also demonstrated an improved metabolic profile compared to benzbromarone. Compound 1 is more stable than benzbromarone in human microsomes. The CYP2C9 metabolic pathway of the compound is significantly reduced and the 6-OH benzbromarone 5,6-di-OH benzbromarone metabolites are not formed. Thus, Compound 1 represents a prospective therapeutic agent for the treatment or prevention of chronic kidney disease with an improved hepatotoxicity profile. In addition, Compound 1 is a prospective therapeutic agent for the treatment or prevention of heart failure.

### Compound 1

In one embodiment is (3,5-dibromo-4-hydroxyphenyl)(2-(1-hydroxyethyl)benzofuran-3-yl-4,5,6,7-d₄)methanone. “Compound 1” or “(3,5-dibromo-4-hydroxyphenyl)(2-(1-hydroxyethyl)benzofuran-3-yl-4,5,6,7-d₄)methanone” refers to the compound with the following structure:

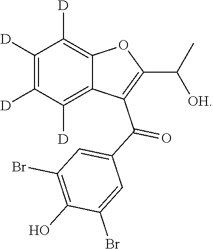 [SMILES: [2H]c1c([2H])c([2H])c2c(C(=O)c3cc(Br)c(O)c(Br)c3)c(C(C)O)oc2c1[2H]]

In some embodiments, Compound 1 includes the solvent addition forms (solvates). Solvates contain either stoichiometric or non-stoichiometric amounts of a solvent, and are formed during the process of product formation or isolation with pharmaceutically acceptable solvents such as water, ethanol, methanol, tert-butyl methyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In some embodiments, solvates are formed using, but not limited to, Class 3 solvent(s). In some embodiments, solvates are formed using, but not limited to, Class 2 solvent(s). Categories of solvents are defined in, for example, the International Conference on Harmonization of Technical Requirements for Registration of Pharmaceuticals for Human Use (ICH), “Impurities: Guidelines for Residual Solvents Q3C(R6),” (October 2016). Hydrates are formed when the solvent is water, or alcoholates are formed when the solvent is alcohol.

In some embodiments, Compound 1 is solvated. In some embodiments, Compound 1 is unsolvated. In some embodiments, Compound 1 is in a pharmaceutically acceptable salt form. In other embodiments, Compound 1 is prepared in various forms, including but not limited to, an amorphous phase, crystalline forms, milled forms, and nano-particulate forms.

### Compound 2

Further described herein is (3,5-dibromo-4-hydroxyphenyl)(6-hydroxy-2-(1-hydroxyethyl)benzofuran-3-yl-4,5,7-d₃)methanone (Compound 2), an active metabolite of (3,5-dibromo-4-hydroxyphenyl)(2-(1-hydroxyethyl)benzofuran-3-yl-4,5,6,7-d₄)methanone (Compound 1).

“Compound 2” or “(3,5-dibromo-4-hydroxyphenyl)(6-hydroxy-2-(1-hydroxyethyl)benzofuran-3-yl-4,5,7-d₃)methanone” refers to the compound with the following structure:

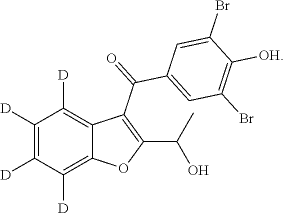 [SMILES: [2H]c1c([2H])c([2H])c2c(C(=O)c3cc(Br)c(O)c(Br)c3)c(C(C)O)oc2c1[2H]]

In some embodiments, Compound 2 includes the solvent addition forms (solvates). Solvates contain either stoichiometric or non-stoichiometric amounts of a solvent, and are formed during the process of product formation or isolation with pharmaceutically acceptable solvents such as water, ethanol, methanol, tert-butyl methyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In some embodiments, solvates are formed using, but not limited to, Class 3 solvent(s). In some embodiments, solvates are formed using, but not limited to, Class 2 solvent(s). Categories of solvents are defined in, for example, the International Conference on Harmonization of Technical Requirements for Registration of Pharmaceuticals for Human Use (ICH), “Impurities: Guidelines for Residual Solvents Q3C(R6),” (October 2016). Hydrates are formed when the solvent is water, or alcoholates are formed when the solvent is alcohol.

In some embodiments, Compound 2 is solvated. In some embodiments, Compound 2 is unsolvated. In some embodiments, Compound 2 is in a pharmaceutically acceptable salt form. In other embodiments, Compound 2 is prepared in various forms, including but not limited to, an amorphous phase, crystalline forms, milled forms, and nano-particulate forms.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy and thermal analysis), as described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features. The term “about” when referring to a number or a numerical range means that the number or numerical range referred to is an approximation within experimental variability (or within statistical experimental error), and thus the number or numerical range may vary between 1% and 15% of the stated number or numerical range.

The section headings used herein are for organizational purposes only and are not to be construed as limiting the subject matter described. All documents, or portions of documents, cited in the application including, but not limited to, patents, patent applications, articles, books, manuals, and treatises are hereby expressly incorporated by reference in their entirety.

The term “acceptable” or “pharmaceutically acceptable”, with respect to a formulation, composition or ingredient, as used herein, means having no persistent detrimental effect on the general health of the subject being treated or does not abrogate the biological activity or properties of the compound, and is relatively nontoxic.

As used herein, “amelioration” of the symptoms of a particular disease, disorder, or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

“Bioavailability” refers to the percentage of Compound 1 dosed that is delivered into the general circulation of the animal or human being studied. The total exposure (AUC_{(0-∞)}) of a drug when administered intravenously is usually defined as 100% bioavailable (F %). “Oral bioavailability” refers to the extent to which Compound 1 is absorbed into the general circulation when the pharmaceutical composition is taken orally as compared to intravenous injection.

“Blood plasma concentration” refers to the concentration of Compound 1 in the plasma component of blood of a subject. It is understood that the plasma concentration of Compound 1 may vary significantly between subjects, due to variability with respect to metabolism and/or possible interactions with other therapeutic agents. In accordance with one embodiment disclosed herein, the blood plasma concentration of Compound 1 may vary from subject to subject. Likewise, values such as maximum plasma concentration (Cₘₐₓ) or time to reach maximum plasma concentration (Tₘₐₓ), or total area under the plasma concentration time curve (AUC_{(0-∞)}) may vary from subject to subject. Due to this variability, the amount necessary to constitute “a therapeutically effective amount” of Compound 1 may vary from subject to subject.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of Compound 1, age, weight, general condition of the subject, the condition being treated, the severity of the condition being treated, and the judgment of the prescribing physician. By way of example only, therapeutically effective amounts may be determined by a dose escalation clinical trial.

The terms “enhance” or “enhancing” means to increase or prolong either in potency or duration a desired effect. By way of example, “enhancing” the effect of therapeutic agents refers to the ability to increase or prolong, either in potency or duration, the effect of therapeutic agents on during treatment of a disease, disorder, or condition. An “enhancing-effective amount,” as used herein, refers to an amount adequate to enhance the effect of a therapeutic agent in the treatment of a disease, disorder, or condition. When used in a patient, amounts effective for this use will depend on the severity and course of the disease, disorder, or condition, previous therapy, the patient's health status and response to the drugs, and the judgment of the treating physician.

The term “prophylactically effective amount,” as used herein, refers that amount of a composition applied to a patient which will relieve to some extent one or more of the symptoms of a disease, condition or disorder being treated. In such prophylactic applications, such amounts may depend on the patient's state of health, weight, and the like. As an example, one can determine such prophylactically effective amounts by a dose escalation clinical trial.

As used herein, the terms “subject”, “patient”, or “individual” is used to mean an animal, preferably a mammal, including a human or non-human. The terms individual, patient and subject may be used interchangeably.

As used herein, the term “target activity” refers to a biological activity capable of being modulated by a selective modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “treat,” “treating” or “treatment”, as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. The terms “treat,” “treating” or “treatment”, include, but are not limited to, prophylactic and/or therapeutic treatments.

As used herein, IC₅₀ refers to a dosage, concentration or amount of a particular test compound that elicits a dose-dependent response at 50% of maximal expression of a particular response that is induced, provoked or potentiated by the particular test compound.

### Pharmaceutical Compositions/Formulations

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

In some embodiments, a pharmaceutical composition, as used herein, refers to a mixture of (3,5-dibromo-4-hydroxyphenyl)(2-(1-hydroxyethyl)benzofuran-3-yl-4,5,6,7-d₄)methanone (Compound 1) or (3,5-dibromo-4-hydroxyphenyl)(6-hydroxy-2-(1-hydroxyethyl)benzofuran-3-yl-4,5,7-d₃)methanone (Compound 2) with other chemical components, such as carriers, stabilizers, diluents, dispersing agents, suspending agents, thickening agents, and/or excipients. The pharmaceutical composition facilitates administration of the compound to a mammal. In practicing the methods of treatment or use provided herein, therapeutically effective amounts of Compound 1 are administered in a pharmaceutical composition to a mammal having a disease, disorder, or condition to be treated. Preferably, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

In some embodiments is a pharmaceutical composition comprising (3,5-dibromo-4-hydroxyphenyl)(2-(1-hydroxyethyl)benzofuran-3-yl-4,5,6,7-d₄)methanone (Compound 1), and at least one inactive ingredient selected from pharmaceutically acceptable carriers, diluents, and excipients.

In some embodiments is a pharmaceutical composition for use in the treatment or prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the treatment of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the treatment or prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the treatment of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients.

In some embodiments is a pharmaceutical composition for use in the treatment or prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the treatment of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1) and at least one inactive ingredient selected from pharmaceutically acceptable carriers, diluents, and excipients. In some embodiments is a pharmaceutical composition for use in the treatment or prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the treatment of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg, and at least one inactive ingredient selected from pharmaceutically acceptable carriers, diluents, and excipients.

In some embodiments is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of hypertension, wherein the pharmaceutical composition comprises a therapeutically effective amount of (3,5-dibromo hydroxyphenyl)(2-(1-hydroxyethyl)benzofuran-3-yl-4,5,6,7-d₄)methanone (Compound 1). In some embodiments is a pharmaceutical composition for use in the treatment of coronary artery disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of Lesch-Nyhan syndrome, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of Kelley-Seegmiller syndrome, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of kidney stones, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of kidney failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of diabetic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of joint inflammation, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of arthritis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of urolithiasis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of plumbism, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of hyperparathyroidism, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of psoriasis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of sarcoidosis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1). In some embodiments is a pharmaceutical composition for use in the treatment of hypoxanthine-guanine phosphoribosyltransferase (HPRT) deficiency, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1).

In some embodiments is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of hypertension, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of coronary artery disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of Lesch-Nyhan syndrome, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of Kelley-Seegmiller syndrome, wherein the pharmaceutical composition comprises a therapeutically effective amount of (3,5-dibromo hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of kidney stones, wherein the pharmaceutical composition comprises a therapeutically effective amount of (3,5-dibromo hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of kidney failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of diabetic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of joint inflammation, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of arthritis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of urolithiasis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of plumbism, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of hyperparathyroidism, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of psoriasis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of sarcoidosis, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a pharmaceutical composition for use in the treatment of hypoxanthine-guanine phosphoribosyltransferase (HPRT) deficiency, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg.

In some embodiments of the pharmaceutical compositions described herein, the therapeutically effective amount of (3,5-dibromo-4-hydroxyphenyl)(2-(1-hydroxyethyl)benzofuran-3-yl-4,5,6,7-d₄)methanone (Compound 1) is from about 3 mg to about 1250 mg. In some embodiments of the pharmaceutical compositions described herein, the therapeutically effective amount of Compound 1 is from about 3 mg to about 1000 mg. In some embodiments of the pharmaceutical compositions described herein, the therapeutically effective amount of Compound 1 is from about 3 mg to about 750 mg. In some embodiments of the pharmaceutical compositions described herein, the therapeutically effective amount of Compound 1 is from about 3 mg to about 600 mg. In some embodiments of the pharmaceutical compositions described herein, the therapeutically effective amount of Compound 1 is from about 3 mg to about 500 mg. In some embodiments of the pharmaceutical compositions described herein, the therapeutically effective amount of Compound 1 is from about 3 mg to about 400 mg. In some embodiments of the pharmaceutical compositions described herein, the therapeutically effective amount of Compound 1 is from about 3 mg to about 300 mg. In some embodiments of the pharmaceutical compositions described herein, the therapeutically effective amount of Compound 1 is from about 3 mg to about 250 mg. In some embodiments of the pharmaceutical compositions described herein, the therapeutically effective amount of Compound 1 is from about 3 mg to about 200 mg. In some embodiments of the pharmaceutical compositions described herein, the therapeutically effective amount of Compound 1 is from about 3 mg to about 150 mg. In some embodiments of the pharmaceutical compositions described herein, the therapeutically effective amount of Compound 1 is from about 3 mg to about 100 mg. In some embodiments of the pharmaceutical compositions described herein, the therapeutically effective amount of Compound 1 is from about 3 mg to about 75 mg. In some embodiments of the pharmaceutical compositions described herein, the therapeutically effective amount of Compound 1 is from about 3 mg to about 50 mg. In some embodiments of the pharmaceutical compositions described herein, the therapeutically effective amount of Compound 1 is from about 3 mg to about 45 mg. In some embodiments of the pharmaceutical compositions described herein, the therapeutically effective amount of Compound 1 is from about 3 mg to about 40 mg. In some embodiments of the pharmaceutical compositions described herein, the therapeutically effective amount of Compound 1 is from about 3 mg to about 35 mg. In some embodiments of the pharmaceutical compositions described herein, the therapeutically effective amount of Compound 1 is from about 3 mg to about 30 mg. In some embodiments of the pharmaceutical compositions described herein, the therapeutically effective amount of Compound 1 is from about 3 mg to about 25 mg. In some embodiments of the pharmaceutical compositions described herein, the therapeutically effective amount of Compound 1 is from about 5 mg to about 300 mg. In some embodiments of the pharmaceutical compositions described herein, the therapeutically effective amount of Compound 1 is from about 5 mg to about 250 mg. In some embodiments of the pharmaceutical compositions described herein, the therapeutically effective amount of Compound 1 is from about 5 mg to about 200 mg. In some embodiments of the pharmaceutical compositions described herein, the therapeutically effective amount of Compound 1 is from about 5 mg to about 150 mg. In some embodiments of the pharmaceutical compositions described herein, the therapeutically effective amount of Compound 1 is from about 5 mg to about 100 mg. In some embodiments of the pharmaceutical compositions described herein, the therapeutically effective amount of Compound 1 is from about 5 mg to about 75 mg. In some embodiments of the pharmaceutical compositions described herein, the therapeutically effective amount of Compound 1 is from about 5 mg to about 50 mg. In some embodiments of the pharmaceutical compositions described herein, the therapeutically effective amount of Compound 1 is from about 5 mg to about 45 mg. In some embodiments of the pharmaceutical compositions described herein, the therapeutically effective amount of Compound 1 is from about 5 mg to about 40 mg. In some embodiments of the pharmaceutical compositions described herein, the therapeutically effective amount of Compound 1 is from about 5 mg to about 35 mg. In some embodiments of the pharmaceutical compositions described herein, the therapeutically effective amount of Compound 1 is from about 5 mg to about 30 mg. In some embodiments of the pharmaceutical compositions described herein, the therapeutically effective amount of Compound 1 is from about 5 mg to about 25 mg. In some embodiments of the pharmaceutical compositions described herein, the therapeutically effective amount of Compound 1 is from about 10 mg to about 200 mg. In some embodiments of the pharmaceutical compositions described herein, the therapeutically effective amount of Compound 1 is from about 10 mg to about 150 mg. In some embodiments of the pharmaceutical compositions described herein, the therapeutically effective amount of Compound 1 is from about 10 mg to about 100 mg. In some embodiments of the pharmaceutical compositions described herein, the therapeutically effective amount of Compound 1 is from about 10 mg to about 75 mg. In some embodiments of the pharmaceutical compositions described herein, the therapeutically effective amount of Compound 1 is from about 25 mg to about 75 mg. In some embodiments of the pharmaceutical compositions described herein, the therapeutically effective amount of Compound 1 is from about 10 mg to about 50 mg. In some embodiments of the pharmaceutical compositions described herein, the therapeutically effective amount of Compound 1 is from about 10 mg to about 45 mg. In some embodiments of the pharmaceutical compositions described herein, the therapeutically effective amount of Compound 1 is from about 10 mg to about 40 mg. In some embodiments of the pharmaceutical compositions described herein, the therapeutically effective amount of Compound 1 is from about 10 mg to about 35 mg. In some embodiments of the pharmaceutical compositions described herein, the therapeutically effective amount of Compound 1 is from about 10 mg to about 30 mg. In some embodiments of the pharmaceutical compositions described herein, the therapeutically effective amount of Compound 1 is from about 10 mg to about 25 mg.

In some embodiments is a pharmaceutical composition comprising (3,5-dibromo-4-hydroxyphenyl)(6-hydroxy-2-(1-hydroxyethyl)benzofuran-3-yl-4,5,7-d₃)methanone (Compound 2), and at least one inactive ingredient selected from pharmaceutically acceptable carriers, diluents, and excipients.

In some embodiments is a pharmaceutical composition for use in the treatment or prevention of chronic kidney disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the treatment of chronic kidney disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the prevention of chronic kidney disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients.

In some embodiments is a pharmaceutical composition for use in the treatment or prevention of heart failure, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the treatment of heart failure, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients. In some embodiments is a pharmaceutical composition for use in the prevention of heart failure, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg, and at least one inactive ingredient selected from pharmaceutically acceptable carriers, diluents, and excipients.

In some embodiments is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of hypertension, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of coronary artery disease, wherein the pharmaceutical composition comprises a therapeutically effective amount of (3,5-dibromo-4-hydroxyphenyl)(6-hydroxy-2-(1-hydroxyethyl)benzofuran yl-4,5,7-d₃)methanone (Compound 2). In some embodiments is a pharmaceutical composition for use in the treatment of Lesch-Nyhan syndrome, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of Kelley-Seegmiller syndrome, wherein the pharmaceutical composition comprises a therapeutically effective amount of (3,5-dibromo hydroxyphenyl)(6-hydroxy-2-(1-hydroxyethyl)benzofuran-3-yl-4,5,7-d₃)methanone (Compound 2). In some embodiments is a pharmaceutical composition for use in the treatment of kidney stones, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of kidney failure, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of diabetic kidney disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of joint inflammation, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of arthritis, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of urolithiasis, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of plumbism, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of hyperparathyroidism, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of psoriasis, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a pharmaceutical composition for use in the treatment of sarcoidosis, wherein the pharmaceutical composition comprises a therapeutically effective amount of (3,5-dibromo hydroxyphenyl)(6-hydroxy-2-(1-hydroxyethyl)benzofuran-3-yl-4,5,7-d₃)methanone (Compound 2). In some embodiments is a pharmaceutical composition for use in the treatment of hypoxanthine-guanine phosphoribosyltransferase (HPRT) deficiency, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2).

In some embodiments is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of hypertension, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of coronary artery disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of Lesch-Nyhan syndrome, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of Kelley-Seegmiller syndrome, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of kidney stones, wherein the pharmaceutical composition comprises a therapeutically effective amount of (3,5-dibromo-4-hydroxyphenyl)(6-hydroxy-2-(1-hydroxyethyl)benzofuran yl-4,5,7-d₃)methanone (Compound 2), wherein the therapeutically effective amount is from about 3 mg to about 1500 mg. In some embodiments is a pharmaceutical composition for use in the treatment of kidney failure, wherein the pharmaceutical composition comprises a therapeutically effective amount of (3,5-dibromo-4-hydroxyphenyl)(6-hydroxy-2-(1-hydroxyethyl)benzofuran yl-4,5,7-d₃)methanone (Compound 2), wherein the therapeutically effective amount is from about 3 mg to about 1500 mg. In some embodiments is a pharmaceutical composition for use in the treatment of diabetic kidney disease, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of joint inflammation, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of arthritis, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of urolithiasis, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of plumbism, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of hyperparathyroidism, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of psoriasis, wherein the pharmaceutical composition comprises a therapeutically effective amount of (3,5-dibromo hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of sarcoidosis, wherein the pharmaceutical composition comprises a therapeutically effective amount of (3,5-dibromo hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a pharmaceutical composition for use in the treatment of hypoxanthine-guanine phosphoribosyltransferase (HPRT) deficiency, wherein the pharmaceutical composition comprises a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg.

In some embodiments of the pharmaceutical compositions described herein, the therapeutically effective amount of (3,5-dibromo-4-hydroxyphenyl)(6-hydroxy-2-(1-hydroxyethyl)benzofuran-3-yl-4,5,7-d₃)methanone (Compound 2) is from about 3 mg to about 1250 mg. In some embodiments of the pharmaceutical compositions described herein, the therapeutically effective amount of Compound 2 is from about 3 mg to about 1000 mg. In some embodiments of the pharmaceutical compositions described herein, the therapeutically effective amount of Compound 2 is from about 3 mg to about 750 mg. In some embodiments of the pharmaceutical compositions described herein, the therapeutically effective amount of Compound 2 is from about 3 mg to about 600 mg. In some embodiments of the pharmaceutical compositions described herein, the therapeutically effective amount of Compound 2 is from about 3 mg to about 500 mg. In some embodiments of the pharmaceutical compositions described herein, the therapeutically effective amount of Compound 2 is from about 3 mg to about 400 mg. In some embodiments of the pharmaceutical compositions described herein, the therapeutically effective amount of Compound 2 is from about 3 mg to about 300 mg. In some embodiments of the pharmaceutical compositions described herein, the therapeutically effective amount of Compound 2 is from about 3 mg to about 250 mg. In some embodiments of the pharmaceutical compositions described herein, the therapeutically effective amount of Compound 2 is from about 3 mg to about 200 mg. In some embodiments of the pharmaceutical compositions described herein, the therapeutically effective amount of Compound 2 is from about 3 mg to about 150 mg. In some embodiments of the pharmaceutical compositions described herein, the therapeutically effective amount of Compound 2 is from about 3 mg to about 100 mg. In some embodiments of the pharmaceutical compositions described herein, the therapeutically effective amount of Compound 2 is from about 3 mg to about 75 mg. In some embodiments of the pharmaceutical compositions described herein, the therapeutically effective amount of Compound 2 is from about 3 mg to about 50 mg. In some embodiments of the pharmaceutical compositions described herein, the therapeutically effective amount of Compound 2 is from about 3 mg to about 45 mg. In some embodiments of the pharmaceutical compositions described herein, the therapeutically effective amount of Compound 2 is from about 3 mg to about 40 mg. In some embodiments of the pharmaceutical compositions described herein, the therapeutically effective amount of Compound 2 is from about 3 mg to about 35 mg. In some embodiments of the pharmaceutical compositions described herein, the therapeutically effective amount of Compound 2 is from about 3 mg to about 30 mg. In some embodiments of the pharmaceutical compositions described herein, the therapeutically effective amount of Compound 2 is from about 3 mg to about 25 mg. In some embodiments of the pharmaceutical compositions described herein, the therapeutically effective amount of Compound 2 is from about 5 mg to about 300 mg. In some embodiments of the pharmaceutical compositions described herein, the therapeutically effective amount of Compound 2 is from about 5 mg to about 250 mg. In some embodiments of the pharmaceutical compositions described herein, the therapeutically effective amount of Compound 2 is from about 5 mg to about 200 mg. In some embodiments of the pharmaceutical compositions described herein, the therapeutically effective amount of Compound 2 is from about 5 mg to about 150 mg. In some embodiments of the pharmaceutical compositions described herein, the therapeutically effective amount of Compound 2 is from about 5 mg to about 100 mg. In some embodiments of the pharmaceutical compositions described herein, the therapeutically effective amount of Compound 2 is from about 5 mg to about 75 mg. In some embodiments of the pharmaceutical compositions described herein, the therapeutically effective amount of Compound 2 is from about 5 mg to about 50 mg. In some embodiments of the pharmaceutical compositions described herein, the therapeutically effective amount of Compound 2 is from about 5 mg to about 45 mg. In some embodiments of the pharmaceutical compositions described herein, the therapeutically effective amount of Compound 2 is from about 5 mg to about 40 mg. In some embodiments of the pharmaceutical compositions described herein, the therapeutically effective amount of Compound 2 is from about 5 mg to about 35 mg. In some embodiments of the pharmaceutical compositions described herein, the therapeutically effective amount of Compound 2 is from about 5 mg to about 30 mg. In some embodiments of the pharmaceutical compositions described herein, the therapeutically effective amount of Compound 2 is from about 5 mg to about 25 mg. In some embodiments of the pharmaceutical compositions described herein, the therapeutically effective amount of Compound 2 is from about 10 mg to about 200 mg. In some embodiments of the pharmaceutical compositions described herein, the therapeutically effective amount of Compound 2 is from about 10 mg to about 150 mg. In some embodiments of the pharmaceutical compositions described herein, the therapeutically effective amount of Compound 2 is from about 10 mg to about 100 mg. In some embodiments of the pharmaceutical compositions described herein, the therapeutically effective amount of Compound 2 is from about 10 mg to about 75 mg. In some embodiments of the pharmaceutical compositions described herein, the therapeutically effective amount of Compound 2 is from about 25 mg to about 75 mg. In some embodiments of the pharmaceutical compositions described herein, the therapeutically effective amount of Compound 2 is from about 10 mg to about 50 mg. In some embodiments of the pharmaceutical compositions described herein, the therapeutically effective amount of Compound 2 is from about 10 mg to about 45 mg. In some embodiments of the pharmaceutical compositions described herein, the therapeutically effective amount of Compound 2 is from about 10 mg to about 40 mg. In some embodiments of the pharmaceutical compositions described herein, the therapeutically effective amount of Compound 2 is from about 10 mg to about 35 mg. In some embodiments of the pharmaceutical compositions described herein, the therapeutically effective amount of Compound 2 is from about 10 mg to about 30 mg. In some embodiments of the pharmaceutical compositions described herein, the therapeutically effective amount of Compound 1 is from about 10 mg to about 25 mg.

In some embodiments is a pharmaceutical composition for use in the treatment or prevention of chronic kidney disease, wherein the pharmaceutical composition comprises a compound selected from:

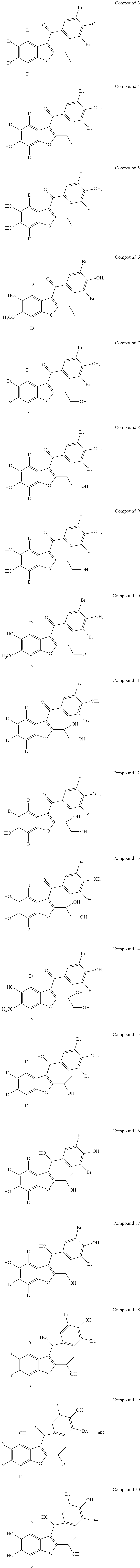

and at least one inactive ingredient selected from pharmaceutically acceptable carriers, diluents, and excipients.

In some embodiments is a pharmaceutical composition for use in the treatment or prevention of heart failure, wherein the pharmaceutical composition comprises a compound selected from:

[chemical structure]

and at least one inactive ingredient selected from pharmaceutically acceptable carriers, diluents, and excipients.

In some embodiments is a pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a compound selected from:

[chemical structure]

and at least one inactive ingredient selected from pharmaceutically acceptable carriers, diluents, and excipients.

In some embodiments of the pharmaceutical compositions described herein, the therapeutically effective amount of any one of Compounds 3-20 is from about 3 mg to about 1500 mg. In some embodiments of the pharmaceutical compositions described herein, the therapeutically effective amount of any one of Compounds 3-20 is from about 3 mg to about 1250 mg. In some embodiments of the pharmaceutical compositions described herein, the therapeutically effective amount of any one of Compounds 3-20 is from about 3 mg to about 1000 mg. In some embodiments of the pharmaceutical compositions described herein, the therapeutically effective amount of any one of Compounds 3-20 is from about 3 mg to about 750 mg. In some embodiments of the pharmaceutical compositions described herein, the therapeutically effective amount of any one of Compounds 3-20 is from about 3 mg to about 600 mg. In some embodiments of the pharmaceutical compositions described herein, the therapeutically effective amount of any one of Compounds 3-20 is from about 3 mg to about 500 mg. In some embodiments of the pharmaceutical compositions described herein, the therapeutically effective amount of any one of Compounds 3-20 is from about 3 mg to about 400 mg. In some embodiments of the pharmaceutical compositions described herein, the therapeutically effective amount of any one of Compounds 3-20 is from about 3 mg to about 300 mg. In some embodiments of the pharmaceutical compositions described herein, the therapeutically effective amount of any one of Compounds 3-20 is from about 3 mg to about 250 mg. In some embodiments of the pharmaceutical compositions described herein, the therapeutically effective amount of any one of Compounds 3-20 is from about 3 mg to about 200 mg. In some embodiments of the pharmaceutical compositions described herein, the therapeutically effective amount of any one of Compounds 3-20 is from about 3 mg to about 150 mg. In some embodiments of the pharmaceutical compositions described herein, the therapeutically effective amount of any one of Compounds 3-20 is from about 3 mg to about 100 mg. In some embodiments of the pharmaceutical compositions described herein, the therapeutically effective amount of any one of Compounds 3-20 is from about 3 mg to about 75 mg. In some embodiments of the pharmaceutical compositions described herein, the therapeutically effective amount of any one of Compounds 3-20 is from about 3 mg to about 50 mg. In some embodiments of the pharmaceutical compositions described herein, the therapeutically effective amount of any one of Compounds 3-20 is from about 3 mg to about 45 mg. In some embodiments of the pharmaceutical compositions described herein, the therapeutically effective amount of any one of Compounds 3-20 is from about 3 mg to about 40 mg. In some embodiments of the pharmaceutical compositions described herein, the therapeutically effective amount of any one of Compounds 3-20 is from about 3 mg to about 35 mg. In some embodiments of the pharmaceutical compositions described herein, the therapeutically effective amount of any one of Compounds 3-20 is from about 3 mg to about 30 mg. In some embodiments of the pharmaceutical compositions described herein, the therapeutically effective amount of any one of Compounds 3-20 is from about 3 mg to about 25 mg. In some embodiments of the pharmaceutical compositions described herein, the therapeutically effective amount of any one of Compounds 3-20 is from about 5 mg to about 300 mg. In some embodiments of the pharmaceutical compositions described herein, the therapeutically effective amount of any one of Compounds 3-20 is from about 5 mg to about 250 mg. In some embodiments of the pharmaceutical compositions described herein, the therapeutically effective amount of any one of Compounds 3-20 is from about 5 mg to about 200 mg. In some embodiments of the pharmaceutical compositions described herein, the therapeutically effective amount of any one of Compounds 3-20 is from about 5 mg to about 150 mg. In some embodiments of the pharmaceutical compositions described herein, the therapeutically effective amount of any one of Compounds 3-20 is from about 5 mg to about 100 mg. In some embodiments of the pharmaceutical compositions described herein, the therapeutically effective amount of any one of Compounds 3-20 is from about 5 mg to about 75 mg. In some embodiments of the pharmaceutical compositions described herein, the therapeutically effective amount of any one of Compounds 3-20 is from about 5 mg to about 50 mg. In some embodiments of the pharmaceutical compositions described herein, the therapeutically effective amount of any one of Compounds 3-20 is from about 5 mg to about 45 mg. In some embodiments of the pharmaceutical compositions described herein, the therapeutically effective amount of any one of Compounds 3-20 is from about 5 mg to about 40 mg. In some embodiments of the pharmaceutical compositions described herein, the therapeutically effective amount of any one of Compounds 3-20 is from about 5 mg to about 35 mg. In some embodiments of the pharmaceutical compositions described herein, the therapeutically effective amount of any one of Compounds 3-20 is from about 5 mg to about 30 mg. In some embodiments of the pharmaceutical compositions described herein, the therapeutically effective amount of any one of Compounds 3-20 is from about 5 mg to about 25 mg. In some embodiments of the pharmaceutical compositions described herein, the therapeutically effective amount of any one of Compounds 3-20 is from about 10 mg to about 200 mg. In some embodiments of the pharmaceutical compositions described herein, the therapeutically effective amount of any one of Compounds 3-20 is from about 10 mg to about 150 mg. In some embodiments of the pharmaceutical compositions described herein, the therapeutically effective amount of any one of Compounds 3-20 is from about 10 mg to about 100 mg. In some embodiments of the pharmaceutical compositions described herein, the therapeutically effective amount of any one of Compounds 3-20 is from about 10 mg to about 75 mg. In some embodiments of the pharmaceutical compositions described herein, the therapeutically effective amount of any one of Compounds 3-20 is from about 25 mg to about 75 mg. In some embodiments of the pharmaceutical compositions described herein, the therapeutically effective amount of any one of Compounds 3-20 is from about 10 mg to about 50 mg. In some embodiments of the pharmaceutical compositions described herein, the therapeutically effective amount of any one of Compounds 3-20 is from about 10 mg to about 45 mg. In some embodiments of the pharmaceutical compositions described herein, the therapeutically effective amount of any one of Compounds 3-20 is from about 10 mg to about 40 mg. In some embodiments of the pharmaceutical compositions described herein, the therapeutically effective amount of any one of Compounds 3-20 is from about 10 mg to about 35 mg. In some embodiments of the pharmaceutical compositions described herein, the therapeutically effective amount of any one of Compounds 3-20 is from about 10 mg to about 30 mg. In some embodiments of the pharmaceutical compositions described herein, the therapeutically effective amount of Compound 1 is from about 10 mg to about 25 mg.

The term “pharmaceutical combination” as used herein, means a product that results from the mixing or combining of more than one active ingredient and includes both fixed and non-fixed combinations of the active ingredients. The term “fixed combination” means that the active ingredients, e.g. Compound 1 or Compound 2, and a co-agent, are both administered to a patient simultaneously in the form of a single entity or dosage. The term “non-fixed combination” means that the active ingredients, e.g. Compound 1 or Compound 2, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

In some embodiments, the compounds described herein are incorporated into pharmaceutical compositions to provide solid oral dosage forms. In other embodiments, the compounds described herein are used to prepare pharmaceutical compositions other than oral solid dosage forms. The pharmaceutical formulations described herein can be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a compound described herein may be manufactured in a conventional manner, such as, by way of example only, by means of conventional mixing, dissolving, granulating, dragee-making, levigating, emulsifying, encapsulating, entrapping or compression processes.

### Methods

In some embodiments is a method for treating or preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating or preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg.

In some embodiments is a method for treating or preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating or preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg.

In some embodiments is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating hypertension in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating coronary artery disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating Lesch-Nyhan syndrome in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating Kelley-Seegmiller syndrome in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating kidney stones in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating kidney failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating diabetic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating joint inflammation in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating arthritis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating urolithiasis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating plumbism in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating hyperparathyroidism in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating psoriasis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating sarcoidosis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1). In some embodiments is a method for treating hypoxanthine-guanine phosphoribosyltransferase (HPRT) deficiency in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1).

In some embodiments is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating hypertension in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating coronary artery disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating Lesch-Nyhan syndrome in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating Kelley-Seegmiller syndrome in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating kidney stones in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating kidney failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating diabetic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating joint inflammation in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating arthritis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating urolithiasis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating plumbism in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating hyperparathyroidism in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating psoriasis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating sarcoidosis in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg. In some embodiments is a method for treating hypoxanthine-guanine phosphoribosyltransferase (HPRT) deficiency in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone (Compound 1), wherein the therapeutically effective amount is from about 3 mg to about 1500 mg.

In some embodiments of the methods described herein, the therapeutically effective amount of (3,5-dibromo-4-hydroxyphenyl)(2-(1-hydroxyethyl)benzofuran-3-yl-4,5,6,7-d₄)methanone (Compound 1) is from about 3 mg to about 1250 mg. In some embodiments of the methods described herein, the therapeutically effective amount of Compound 1 is from about 3 mg to about 1000 mg. In some embodiments of the methods described herein, the therapeutically effective amount of Compound 1 is from about 3 mg to about 750 mg. In some embodiments of the methods described herein, the therapeutically effective amount of Compound 1 is from about 3 mg to about 600 mg. In some embodiments of the methods described herein, the therapeutically effective amount of Compound 1 is from about 3 mg to about 500 mg. In some embodiments of the methods described herein, the therapeutically effective amount of Compound 1 is from about 3 mg to about 400 mg. In some embodiments of the methods described herein, the therapeutically effective amount of Compound 1 is from about 3 mg to about 300 mg. In some embodiments of the methods described herein, the therapeutically effective amount of Compound 1 is from about 3 mg to about 250 mg. In some embodiments of the methods described herein, the therapeutically effective amount of Compound 1 is from about 3 mg to about 200 mg. In some embodiments of the methods described herein, the therapeutically effective amount of Compound 1 is from about 3 mg to about 150 mg. In some embodiments of the methods described herein, the therapeutically effective amount of Compound 1 is from about 3 mg to about 100 mg. In some embodiments of the methods described herein, the therapeutically effective amount of Compound 1 is from about 3 mg to about 75 mg. In some embodiments of the methods described herein, the therapeutically effective amount of Compound 1 is from about 3 mg to about 50 mg. In some embodiments of the methods described herein, the therapeutically effective amount of Compound 1 is from about 3 mg to about 45 mg. In some embodiments of the methods described herein, the therapeutically effective amount of Compound 1 is from about 3 mg to about 40 mg. In some embodiments of the methods described herein, the therapeutically effective amount of Compound 1 is from about 3 mg to about 35 mg. In some embodiments of the methods described herein, the therapeutically effective amount of Compound 1 is from about 3 mg to about 30 mg. In some embodiments of the methods described herein, the therapeutically effective amount of Compound 1 is from about 3 mg to about 25 mg. In some embodiments of the methods described herein, the therapeutically effective amount of Compound 1 is from about 5 mg to about 300 mg. In some embodiments of the methods described herein, the therapeutically effective amount of Compound 1 is from about 5 mg to about 250 mg. In some embodiments of the methods described herein, the therapeutically effective amount of Compound 1 is from about 5 mg to about 200 mg. In some embodiments of the methods described herein, the therapeutically effective amount of Compound 1 is from about 5 mg to about 150 mg. In some embodiments of the methods described herein, the therapeutically effective amount of Compound 1 is from about 5 mg to about 100 mg. In some embodiments of the methods described herein, the therapeutically effective amount of Compound 1 is from about 5 mg to about 75 mg. In some embodiments of the methods described herein, the therapeutically effective amount of Compound 1 is from about 5 mg to about 50 mg. In some embodiments of the methods described herein, the therapeutically effective amount of Compound 1 is from about 5 mg to about 45 mg. In some embodiments of the methods described herein, the therapeutically effective amount of Compound 1 is from about 5 mg to about 40 mg. In some embodiments of the methods described herein, the therapeutically effective amount of Compound 1 is from about 5 mg to about 35 mg. In some embodiments of the methods described herein, the therapeutically effective amount of Compound 1 is from about 5 mg to about 30 mg. In some embodiments of the methods described herein, the therapeutically effective amount of Compound 1 is from about 5 mg to about 25 mg. In some embodiments of the methods described herein, the therapeutically effective amount of Compound 1 is from about 10 mg to about 200 mg. In some embodiments of the methods described herein, the therapeutically effective amount of Compound 1 is from about 10 mg to about 150 mg. In some embodiments of the methods described herein, the therapeutically effective amount of Compound 1 is from about 10 mg to about 100 mg. In some embodiments of the methods described herein, the therapeutically effective amount of Compound 1 is from about 10 mg to about 75 mg. In some embodiments of the methods described herein, the therapeutically effective amount of Compound 1 is from about 25 mg to about 75 mg. In some embodiments of the methods described herein, the therapeutically effective amount of Compound 1 is from about 10 mg to about 50 mg. In some embodiments of the methods described herein, the therapeutically effective amount of Compound 1 is from about 10 mg to about 45 mg. In some embodiments of the methods described herein, the therapeutically effective amount of Compound 1 is from about 10 mg to about 40 mg. In some embodiments of the methods described herein, the therapeutically effective amount of Compound 1 is from about 10 mg to about 35 mg. In some embodiments of the methods described herein, the therapeutically effective amount of Compound 1 is from about 10 mg to about 30 mg. In some embodiments of the methods described herein, the therapeutically effective amount of Compound 1 is from about 10 mg to about 25 mg.

In some embodiments is a method for treating or preventing chronic kidney disease comprising administering to the individual in need thereof a therapeutically effective amount of (3,5-dibromo-4-hydroxyphenyl)(6-hydroxy-2-(1-hydroxyethyl)benzofuran-3-yl-4,5,7-d₃)methanone (Compound 2) described herein. In some embodiments is a method for treating or preventing chronic kidney disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating chronic kidney disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for preventing chronic kidney disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg.

In some embodiments is a method for treating or preventing heart failure comprising administering to the individual in need thereof a therapeutically effective amount of (3,5-dibromo-4-hydroxyphenyl)(6-hydroxy-2-(1-hydroxyethyl)benzofuran-3-yl-4,5,7-d₃)methanone (Compound 2) described herein. In some embodiments is a method for treating or preventing heart failure in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating heart failure in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for preventing heart failure in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg.

In some embodiments is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating hypertension in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating coronary artery disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating Lesch-Nyhan syndrome in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating Kelley-Seegmiller syndrome in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating kidney stones in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating kidney failure in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating diabetic kidney disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating joint inflammation in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating arthritis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating urolithiasis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating plumbism in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating hyperparathyroidism in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating psoriasis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating sarcoidosis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2). In some embodiments is a method for treating hypoxanthine-guanine phosphoribosyltransferase (HPRT) deficiency in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2).

In some embodiments is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating hypertension in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating coronary artery disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating Lesch-Nyhan syndrome in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating Kelley-Seegmiller syndrome in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating kidney stones in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating kidney failure in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating diabetic kidney disease in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating joint inflammation in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating arthritis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating urolithiasis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating plumbism in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating hyperparathyroidism in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating psoriasis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating sarcoidosis in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg. In some embodiments is a method for treating hypoxanthine-guanine phosphoribosyltransferase (HPRT) deficiency in an individual in need thereof, comprising administering to the individual a therapeutically effective amount of (3,5-dibromo-4-hydroxyphenyl)(6-hydroxy-2-(1-hydroxyethyl)benzofuran-3-yl-4,5,7-d₃)methanone (Compound 2), wherein the therapeutically effective amount is from about 3 mg to about 1500 mg.

In some embodiments of the methods described herein, the therapeutically effective amount of (3,5-dibromo-4-hydroxyphenyl)(6-hydroxy-2-(1-hydroxyethyl)benzofuran-3-yl-4,5,7-d₃)methanone (Compound 2) is from about 3 mg to about 1250 mg. In some embodiments of the methods described herein, the therapeutically effective amount of Compound 2 is from about 3 mg to about 1000 mg. In some embodiments of the methods described herein, the therapeutically effective amount of Compound 2 is from about 3 mg to about 750 mg. In some embodiments of the methods described herein, the therapeutically effective amount of Compound 2 is from about 3 mg to about 600 mg. In some embodiments of the methods described herein, the therapeutically effective amount of Compound 2 is from about 3 mg to about 500 mg. In some embodiments of the methods described herein, the therapeutically effective amount of Compound 2 is from about 3 mg to about 400 mg. In some embodiments of the methods described herein, the therapeutically effective amount of Compound 2 is from about 3 mg to about 300 mg. In some embodiments of the methods described herein, the therapeutically effective amount of Compound 2 is from about 3 mg to about 250 mg. In some embodiments of the methods described herein, the therapeutically effective amount of Compound 2 is from about 3 mg to about 200 mg. In some embodiments of the methods described herein, the therapeutically effective amount of Compound 2 is from about 3 mg to about 150 mg. In some embodiments of the methods described herein, the therapeutically effective amount of Compound 2 is from about 3 mg to about 100 mg. In some embodiments of the methods described herein, the therapeutically effective amount of Compound 2 is from about 3 mg to about 75 mg. In some embodiments of the methods described herein, the therapeutically effective amount of Compound 2 is from about 3 mg to about 50 mg. In some embodiments of the methods described herein, the therapeutically effective amount of Compound 2 is from about 3 mg to about 45 mg. In some embodiments of the methods described herein, the therapeutically effective amount of Compound 2 is from about 3 mg to about 40 mg. In some embodiments of the methods described herein, the therapeutically effective amount of Compound 2 is from about 3 mg to about 35 mg. In some embodiments of the methods described herein, the therapeutically effective amount of Compound 2 is from about 3 mg to about 30 mg. In some embodiments of the methods described herein, the therapeutically effective amount of Compound 2 is from about 3 mg to about 25 mg. In some embodiments of the methods described herein, the therapeutically effective amount of Compound 2 is from about 5 mg to about 300 mg. In some embodiments of the methods described herein, the therapeutically effective amount of Compound 2 is from about 5 mg to about 250 mg. In some embodiments of the methods described herein, the therapeutically effective amount of Compound 2 is from about 5 mg to about 200 mg. In some embodiments of the methods described herein, the therapeutically effective amount of Compound 2 is from about 5 mg to about 150 mg. In some embodiments of the methods described herein, the therapeutically effective amount of Compound 2 is from about 5 mg to about 100 mg. In some embodiments of the methods described herein, the therapeutically effective amount of Compound 2 is from about 5 mg to about 75 mg. In some embodiments of the methods described herein, the therapeutically effective amount of Compound 2 is from about 5 mg to about 50 mg. In some embodiments of the methods described herein, the therapeutically effective amount of Compound 2 is from about 5 mg to about 45 mg. In some embodiments of the methods described herein, the therapeutically effective amount of Compound 2 is from about 5 mg to about 40 mg. In some embodiments of the methods described herein, the therapeutically effective amount of Compound 2 is from about 5 mg to about 35 mg. In some embodiments of the methods described herein, the therapeutically effective amount of Compound 2 is from about 5 mg to about 30 mg. In some embodiments of the methods described herein, the therapeutically effective amount of Compound 2 is from about 5 mg to about 25 mg. In some embodiments of the methods described herein, the therapeutically effective amount of Compound 2 is from about 10 mg to about 200 mg. In some embodiments of the methods described herein, the therapeutically effective amount of Compound 2 is from about 10 mg to about 150 mg. In some embodiments of the methods described herein, the therapeutically effective amount of Compound 2 is from about 10 mg to about 100 mg. In some embodiments of the methods described herein, the therapeutically effective amount of Compound 2 is from about 10 mg to about 75 mg. In some embodiments of the methods described herein, the therapeutically effective amount of Compound 2 is from about 25 mg to about 75 mg. In some embodiments of the methods described herein, the therapeutically effective amount of Compound 2 is from about 10 mg to about 50 mg. In some embodiments of the methods described herein, the therapeutically effective amount of Compound 2 is from about 10 mg to about 45 mg. In some embodiments of the methods described herein, the therapeutically effective amount of Compound 2 is from about 10 mg to about 40 mg. In some embodiments of the methods described herein, the therapeutically effective amount of Compound 2 is from about 10 mg to about 35 mg. In some embodiments of the methods described herein, the therapeutically effective amount of Compound 2 is from about 10 mg to about 30 mg. In some embodiments of the methods described herein, the therapeutically effective amount of Compound 2 is from about 10 mg to about 25 mg.

In some embodiments described herein is a method for treating or preventing chronic kidney disease comprising administering to the individual in need thereof a therapeutically effective amount of a compound selected from:

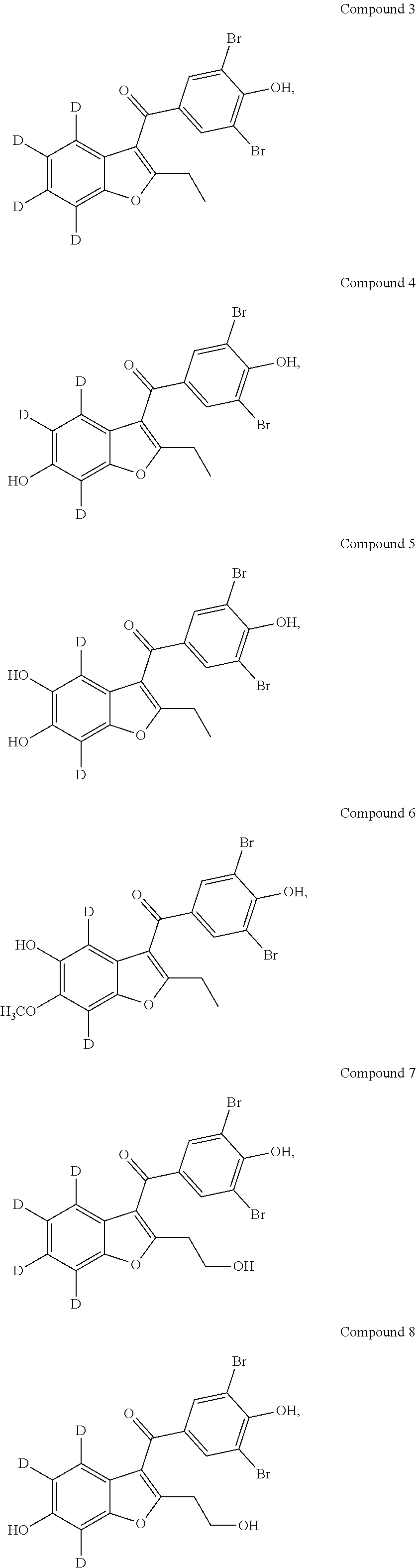 [SMILES: [2H]c1c([2H])c([2H])c2c(C(=O)c3cc(Br)c(O)c(Br)c3)c(CC)oc2c1[2H] ; [2H]c1c(O)c([2H])c2oc(CC)c(C(=O)c3cc(Br)c(O)c(Br)c3)c2c1[2H] ; [2H]c1c(O)c(O)c([2H])c2c(C(=O)c3cc(Br)c(O)c(Br)c3)c(CC)oc12 ; [2H]c1c(OC)c(O)c([2H])c2c(C(=O)c3cc(Br)c(O)c(Br)c3)c(CC)oc12 ; [2H]c1c([2H])c([2H])c2c(C(=O)c3cc(Br)c(O)c(Br)c3)c(CCO)oc2c1[2H] ; [2H]c1c(O)c([2H])c2oc(CCO)c(C(=O)c3cc(Br)c(O)c(Br)c3)c2c1[2H]]

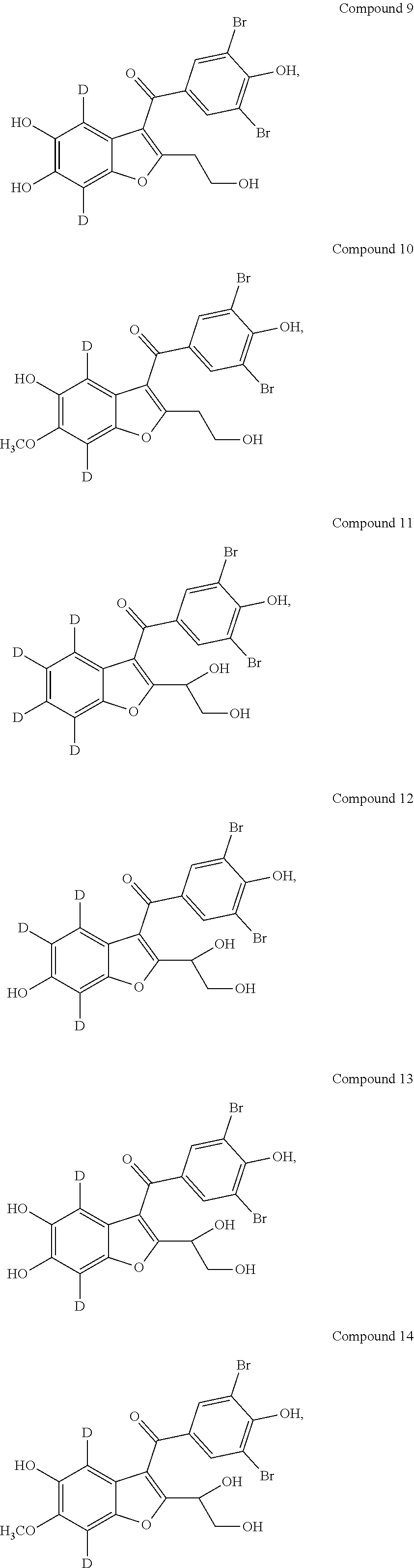 [SMILES: [2H]c1c(O)c(O)c([2H])c2c(C(=O)c3cc(Br)c(O)c(Br)c3)c(CCO)oc12 ; [2H]c1c(OC)c(O)c([2H])c2c(C(=O)c3cc(Br)c(O)c(Br)c3)c(CCO)oc12 ; [2H]c1c([2H])c([2H])c2c(C(=O)c3cc(Br)c(O)c(Br)c3)c(C(O)CO)oc2c1[2H] ; [2H]c1c(O)c([2H])c2oc(C(O)CO)c(C(=O)c3cc(Br)c(O)c(Br)c3)c2c1[2H] ; [2H]c1c(O)c(O)c([2H])c2c(C(=O)c3cc(Br)c(O)c(Br)c3)c(C(O)CO)oc12 ; [2H]c1c(OC)c(O)c([2H])c2c(C(=O)c3cc(Br)c(O)c(Br)c3)c(C(O)CO)oc12]

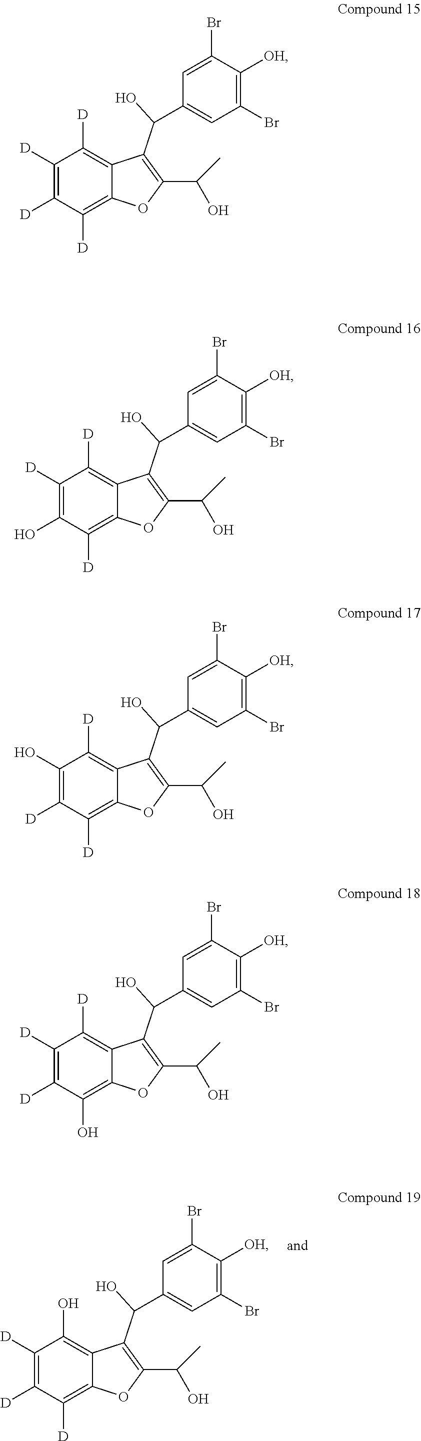 [SMILES: [2H]c1c([2H])c([2H])c2c(C(O)c3cc(Br)c(O)c(Br)c3)c(C(C)O)oc2c1[2H] ; [2H]c1c(O)c([2H])c2oc(C(C)O)c(C(O)c3cc(Br)c(O)c(Br)c3)c2c1[2H] ; [2H]c1c(O)c([2H])c2c(C(O)c3cc(Br)c(O)c(Br)c3)c(C(C)O)oc2c1[2H] ; [2H]c1c([2H])c([2H])c2c(C(O)c3cc(Br)c(O)c(Br)c3)c(C(C)O)oc2c1O ; [2H]c1c([2H])c(O)c2c(C(O)c3cc(Br)c(O)c(Br)c3)c(C(C)O)oc2c1[2H] ; "and"]

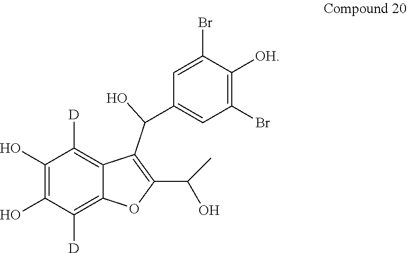 [SMILES: [2H]c1c(O)c(O)c([2H])c2c(C(O)c3cc(Br)c(O)c(Br)c3)c(C(C)O)oc12]

In some embodiments described herein is a method for treating or preventing heart failure comprising administering to the individual in need thereof a therapeutically effective amount of a compound selected from:

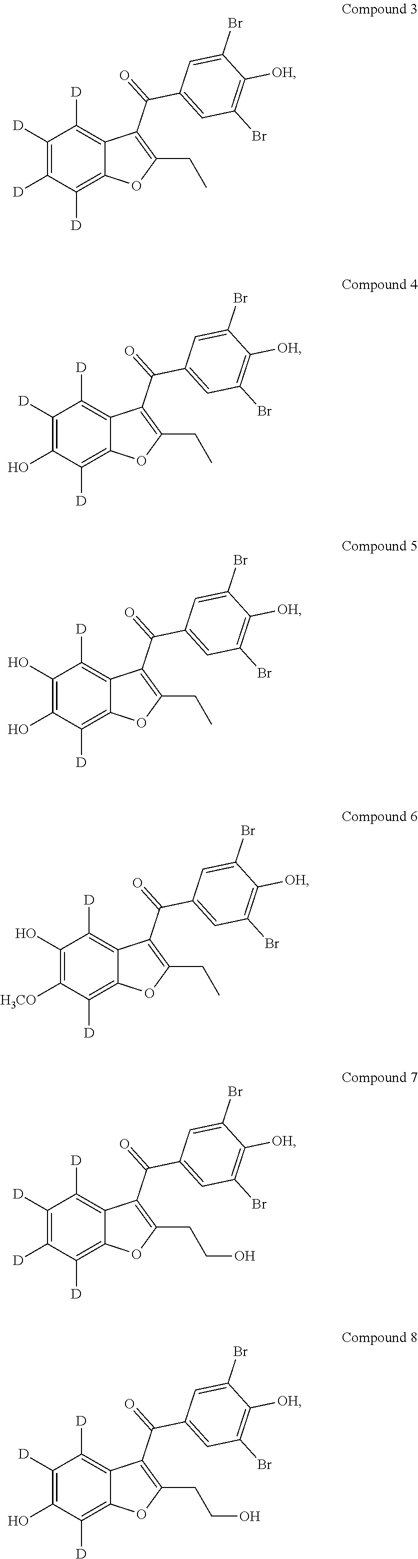 [SMILES: [2H]c1c([2H])c([2H])c2c(C(=O)c3cc(Br)c(O)c(Br)c3)c(CC)oc2c1[2H] ; [2H]c1c(O)c([2H])c2oc(CC)c(C(=O)c3cc(Br)c(O)c(Br)c3)c2c1[2H] ; [2H]c1c(O)c(O)c([2H])c2c(C(=O)c3cc(Br)c(O)c(Br)c3)c(CC)oc12 ; [2H]c1c(OC)c(O)c([2H])c2c(C(=O)c3cc(Br)c(O)c(Br)c3)c(CC)oc12 ; [2H]c1c([2H])c([2H])c2c(C(=O)c3cc(Br)c(O)c(Br)c3)c(CCO)oc2c1[2H] ; [2H]c1c(O)c([2H])c2oc(CCO)c(C(=O)c3cc(Br)c(O)c(Br)c3)c2c1[2H]]

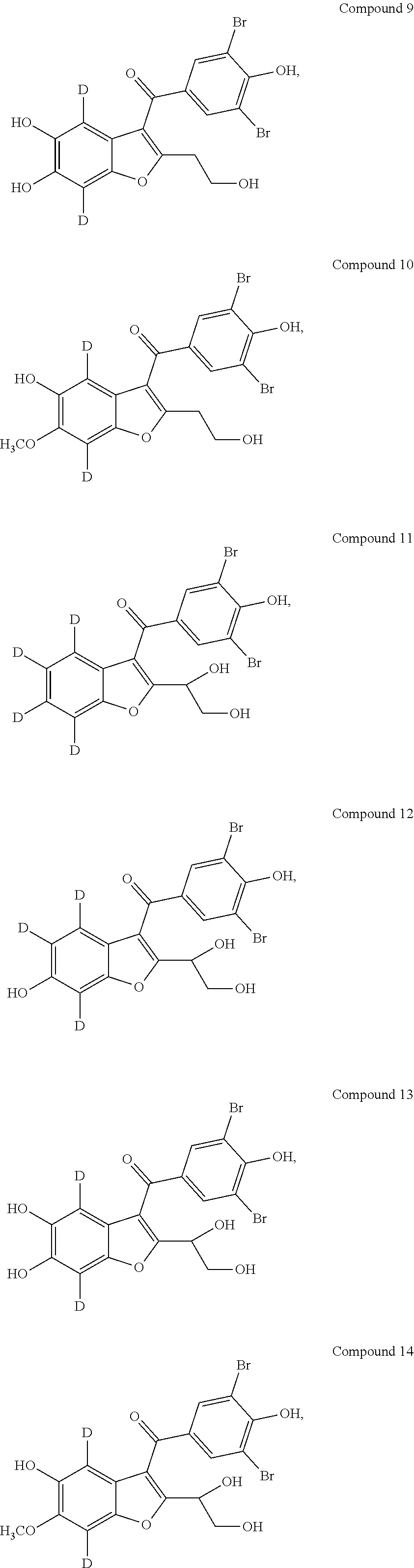 [SMILES: [2H]c1c(O)c(O)c([2H])c2c(C(=O)c3cc(Br)c(O)c(Br)c3)c(CCO)oc12 ; [2H]c1c(OC)c(O)c([2H])c2c(C(=O)c3cc(Br)c(O)c(Br)c3)c(CCO)oc12 ; [2H]c1c([2H])c([2H])c2c(C(=O)c3cc(Br)c(O)c(Br)c3)c(C(O)CO)oc2c1[2H] ; [2H]c1c(O)c([2H])c2oc(C(O)CO)c(C(=O)c3cc(Br)c(O)c(Br)c3)c2c1[2H] ; [2H]c1c(O)c(O)c([2H])c2c(C(=O)c3cc(Br)c(O)c(Br)c3)c(C(O)CO)oc12 ; [2H]c1c(OC)c(O)c([2H])c2c(C(=O)c3cc(Br)c(O)c(Br)c3)c(C(O)CO)oc12]

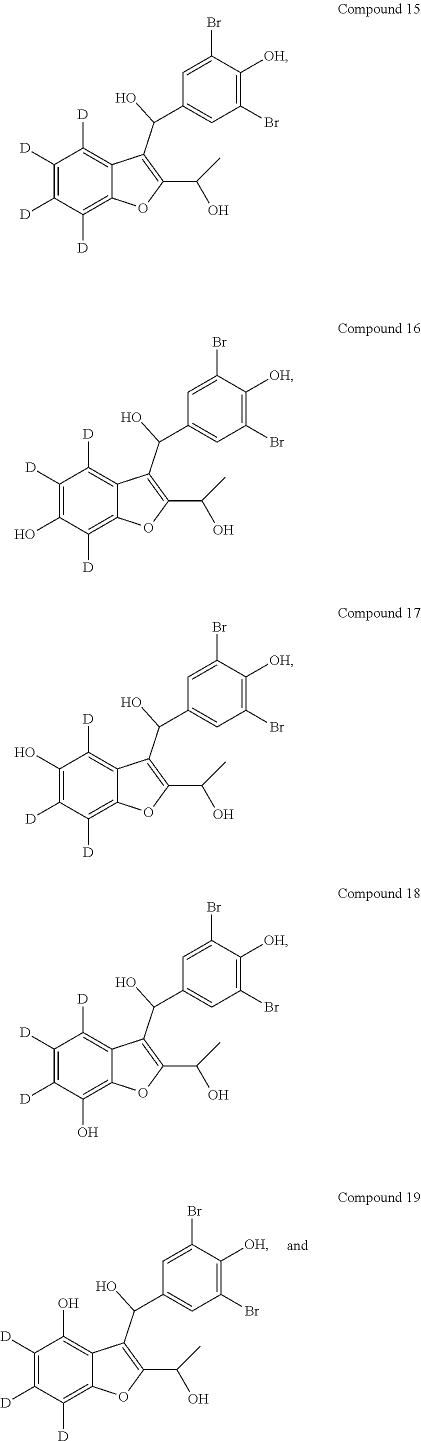 [SMILES: [2H]c1c([2H])c([2H])c2c(C(O)c3cc(Br)c(O)c(Br)c3)c(C(C)O)oc2c1[2H] ; [2H]c1c(O)c([2H])c2oc(C(C)O)c(C(O)c3cc(Br)c(O)c(Br)c3)c2c1[2H] ; [2H]c1c(O)c([2H])c2c(C(O)c3cc(Br)c(O)c(Br)c3)c(C(C)O)oc2c1[2H] ; [2H]c1c([2H])c([2H])c2c(C(O)c3cc(Br)c(O)c(Br)c3)c(C(C)O)oc2c1O ; [2H]c1c([2H])c(O)c2c(C(O)c3cc(Br)c(O)c(Br)c3)c(C(C)O)oc2c1[2H] ; "and"]

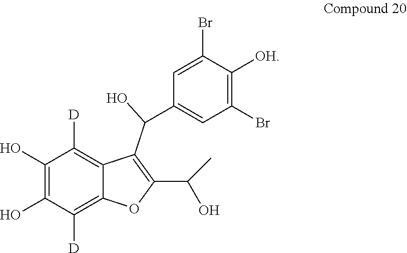 [SMILES: [2H]c1c(O)c(O)c([2H])c2c(C(O)c3cc(Br)c(O)c(Br)c3)c(C(C)O)oc12]

In some embodiments described herein is a method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, comprising administering to the individual in need thereof a therapeutically effective amount of a compound selected from:

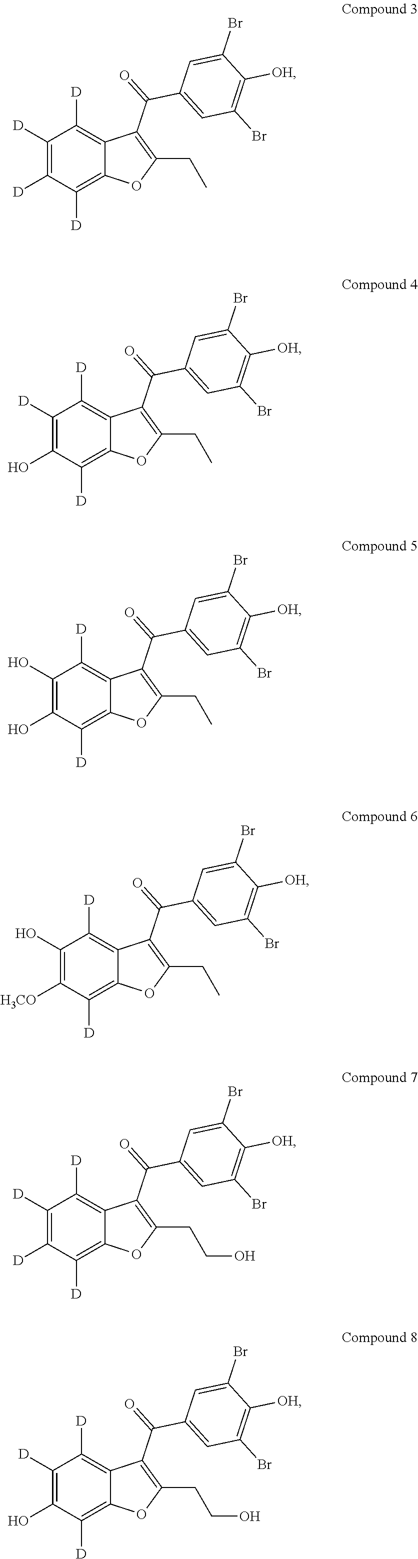 [SMILES: [2H]c1c([2H])c([2H])c2c(C(=O)c3cc(Br)c(O)c(Br)c3)c(CC)oc2c1[2H] ; [2H]c1c(O)c([2H])c2oc(CC)c(C(=O)c3cc(Br)c(O)c(Br)c3)c2c1[2H] ; [2H]c1c(O)c(O)c([2H])c2c(C(=O)c3cc(Br)c(O)c(Br)c3)c(CC)oc12 ; [2H]c1c(OC)c(O)c([2H])c2c(C(=O)c3cc(Br)c(O)c(Br)c3)c(CC)oc12 ; [2H]c1c([2H])c([2H])c2c(C(=O)c3cc(Br)c(O)c(Br)c3)c(CCO)oc2c1[2H] ; [2H]c1c(O)c([2H])c2oc(CCO)c(C(=O)c3cc(Br)c(O)c(Br)c3)c2c1[2H]]

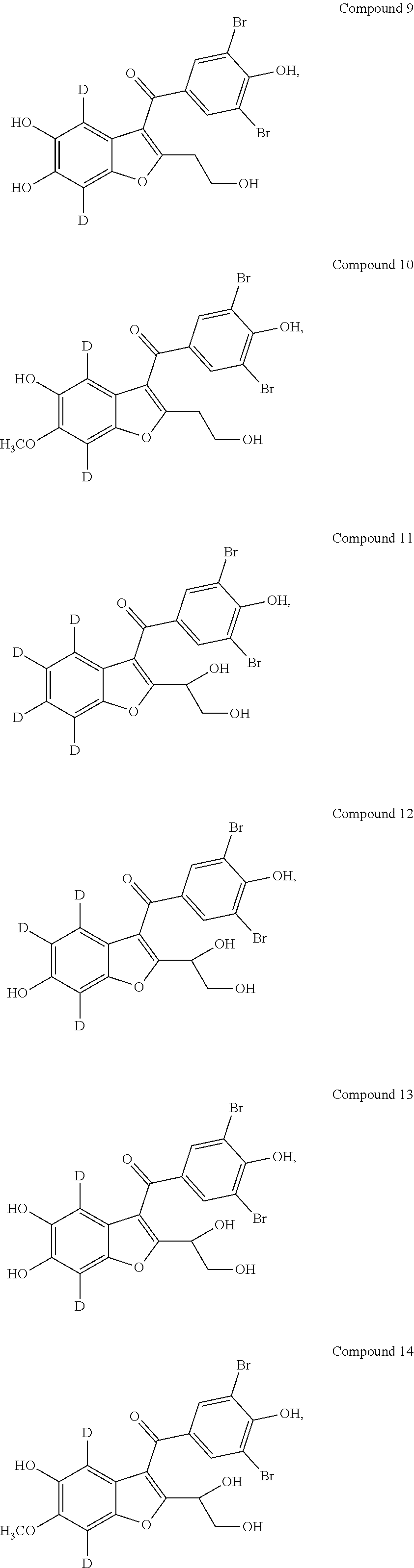 [SMILES: [2H]c1c(O)c(O)c([2H])c2c(C(=O)c3cc(Br)c(O)c(Br)c3)c(CCO)oc12 ; [2H]c1c(OC)c(O)c([2H])c2c(C(=O)c3cc(Br)c(O)c(Br)c3)c(CCO)oc12 ; [2H]c1c([2H])c([2H])c2c(C(=O)c3cc(Br)c(O)c(Br)c3)c(C(O)CO)oc2c1[2H] ; [2H]c1c(O)c([2H])c2oc(C(O)CO)c(C(=O)c3cc(Br)c(O)c(Br)c3)c2c1[2H] ; [2H]c1c(O)c(O)c([2H])c2c(C(=O)c3cc(Br)c(O)c(Br)c3)c(C(O)CO)oc12 ; [2H]c1c(OC)c(O)c([2H])c2c(C(=O)c3cc(Br)c(O)c(Br)c3)c(C(O)CO)oc12]

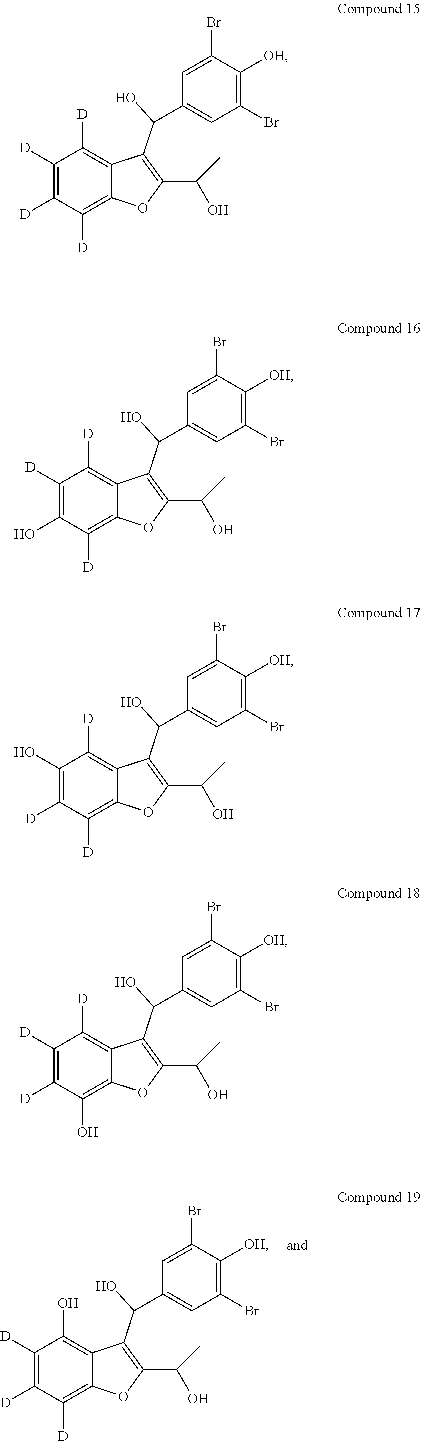 [SMILES: [2H]c1c([2H])c([2H])c2c(C(O)c3cc(Br)c(O)c(Br)c3)c(C(C)O)oc2c1[2H] ; [2H]c1c(O)c([2H])c2oc(C(C)O)c(C(O)c3cc(Br)c(O)c(Br)c3)c2c1[2H] ; [2H]c1c(O)c([2H])c2c(C(O)c3cc(Br)c(O)c(Br)c3)c(C(C)O)oc2c1[2H] ; [2H]c1c([2H])c([2H])c2c(C(O)c3cc(Br)c(O)c(Br)c3)c(C(C)O)oc2c1O ; [2H]c1c([2H])c(O)c2c(C(O)c3cc(Br)c(O)c(Br)c3)c(C(C)O)oc2c1[2H] ; "and"]

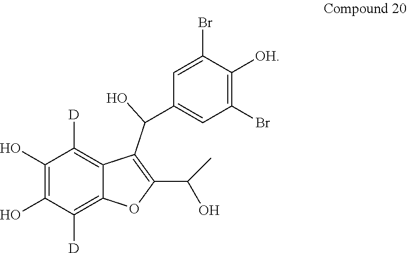 [SMILES: [2H]c1c(O)c(O)c([2H])c2c(C(O)c3cc(Br)c(O)c(Br)c3)c(C(C)O)oc12]

In some embodiments of the methods described herein, the therapeutically effective amount of any one of Compounds 3-20 is from about 3 mg to about 1500 mg. In some embodiments of the methods described herein, the therapeutically effective amount of any one of Compounds 3-20 is from about 3 mg to about 1250 mg. In some embodiments of the methods described herein, the therapeutically effective amount of any one of Compounds 3-20 is from about 3 mg to about 1000 mg. In some embodiments of the methods described herein, the therapeutically effective amount of any one of Compounds 3-20 is from about 3 mg to about 750 mg. In some embodiments of the methods described herein, the therapeutically effective amount of any one of Compounds 3-20 is from about 3 mg to about 600 mg. In some embodiments of the methods described herein, the therapeutically effective amount of any one of Compounds 3-20 is from about 3 mg to about 500 mg. In some embodiments of the methods described herein, the therapeutically effective amount of any one of Compounds 3-20 is from about 3 mg to about 400 mg. In some embodiments of the methods described herein, the therapeutically effective amount of any one of Compounds 3-20 is from about 3 mg to about 300 mg. In some embodiments of the methods described herein, the therapeutically effective amount of any one of Compounds 3-20 is from about 3 mg to about 250 mg. In some embodiments of the methods described herein, the therapeutically effective amount of any one of Compounds 3-20 is from about 3 mg to about 200 mg. In some embodiments of the methods described herein, the therapeutically effective amount of any one of Compounds 3-20 is from about 3 mg to about 150 mg. In some embodiments of the methods described herein, the therapeutically effective amount of any one of Compounds 3-20 is from about 3 mg to about 100 mg. In some embodiments of the methods described herein, the therapeutically effective amount of any one of Compounds 3-20 is from about 3 mg to about 75 mg. In some embodiments of the methods described herein, the therapeutically effective amount of any one of Compounds 3-20 is from about 3 mg to about 50 mg. In some embodiments of the methods described herein, the therapeutically effective amount of any one of Compounds 3-20 is from about 3 mg to about 45 mg. In some embodiments of the methods described herein, the therapeutically effective amount of any one of Compounds 3-20 is from about 3 mg to about 40 mg. In some embodiments of the methods described herein, the therapeutically effective amount of any one of Compounds 3-20 is from about 3 mg to about 35 mg. In some embodiments of the methods described herein, the therapeutically effective amount of any one of Compounds 3-20 is from about 3 mg to about 30 mg. In some embodiments of the methods described herein, the therapeutically effective amount of any one of Compounds 3-20 is from about 3 mg to about 25 mg. In some embodiments of the methods described herein, the therapeutically effective amount of any one of Compounds 3-20 is from about 5 mg to about 300 mg. In some embodiments of the methods described herein, the therapeutically effective amount of any one of Compounds 3-20 is from about 5 mg to about 250 mg. In some embodiments of the methods described herein, the therapeutically effective amount of any one of Compounds 3-20 is from about 5 mg to about 200 mg. In some embodiments of the methods described herein, the therapeutically effective amount of any one of Compounds 3-20 is from about 5 mg to about 150 mg. In some embodiments of the methods described herein, the therapeutically effective amount of any one of Compounds 3-20 is from about 5 mg to about 100 mg. In some embodiments of the methods described herein, the therapeutically effective amount of any one of Compounds 3-20 is from about 5 mg to about 75 mg. In some embodiments of the methods described herein, the therapeutically effective amount of any one of Compounds 3-20 is from about 5 mg to about 50 mg. In some embodiments of the methods described herein, the therapeutically effective amount of any one of Compounds 3-20 is from about 5 mg to about 45 mg. In some embodiments of the methods described herein, the therapeutically effective amount of any one of Compounds 3-20 is from about 5 mg to about 40 mg. In some embodiments of the methods described herein, the therapeutically effective amount of any one of Compounds 3-20 is from about 5 mg to about 35 mg. In some embodiments of the methods described herein, the therapeutically effective amount of any one of Compounds 3-20 is from about 5 mg to about 30 mg. In some embodiments of the methods described herein, the therapeutically effective amount of any one of Compounds 3-20 is from about 5 mg to about 25 mg. In some embodiments of the methods described herein, the therapeutically effective amount of any one of Compounds 3-20 is from about 10 mg to about 200 mg. In some embodiments of the methods described herein, the therapeutically effective amount of any one of Compounds 3-20 is from about 10 mg to about 150 mg. In some embodiments of the methods described herein, the therapeutically effective amount of any one of Compounds 3-20 is from about 10 mg to about 100 mg. In some embodiments of the methods described herein, the therapeutically effective amount of any one of Compounds 3-20 is from about 10 mg to about 75 mg. In some embodiments of the methods described herein, the therapeutically effective amount of any one of Compounds 3-20 is from about 25 mg to about 75 mg. In some embodiments of the methods described herein, the therapeutically effective amount of any one of Compounds 3-20 is from about 10 mg to about 50 mg. In some embodiments of the methods described herein, the therapeutically effective amount of any one of Compounds 3-20 is from about 10 mg to about 45 mg. In some embodiments of the methods described herein, the therapeutically effective amount of any one of Compounds 3-20 is from about 10 mg to about 40 mg. In some embodiments of the methods described herein, the therapeutically effective amount of any one of Compounds 3-20 is from about 10 mg to about 35 mg. In some embodiments of the methods described herein, the therapeutically effective amount of any one of Compounds 3-20 is from about 10 mg to about 30 mg. In some embodiments of the methods described herein, the therapeutically effective amount of any one of Compounds 3-20 is from about 10 mg to about 25 mg.

### Methods of Dosing and Treatment Regimens

In some embodiments, Compound 1 or Compound 2 is used in the preparation of medicaments for the treatment of diseases or conditions that would benefit from lowering serum uric acid (sUA). In addition, a method for treating any of the diseases or conditions described herein in an individual in need of such treatment, involves administration of pharmaceutical compositions containing Compound 1 or Compound 2, or pharmaceutically acceptable solvate thereof, in therapeutically effective amounts to said individual.

In some embodiments, compositions containing Compound 1 or Compound 2 are administered for prophylactic, therapeutic, or maintenance treatment. In some embodiments, compositions containing Compound 1 or Compound 2 are administered for therapeutic applications. In some embodiments, compositions containing Compound 1 or Compound 2 are administered for prophylactic applications.

In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder, or condition, previous therapy, the patient's health status and response to the drugs, and the judgment of the treating physician.

In some embodiments, Compound 1 or Compound 2 is administered daily. In some embodiments, Compound 1 or Compound 2 is administered every other day.

In some embodiments, Compound 1 or Compound 2 is administered once per day. In some embodiments, Compound 1 or Compound 2 is administered twice per day. In some embodiments, Compound 1 or Compound 2 is administered three times per day. In some embodiments, Compound 1 or Compound 2 is administered four times per day.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered, if necessary. Subsequently, the dosage or the frequency of administration, or both, can be reduced, as a function of the symptoms, to a level at which the improved disease, disorder, or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

The daily dosages appropriate for the compounds described herein are from about 0.01 mg/kg to about 20 mg/kg. In one embodiment, the daily dosages are from about 0.1 mg/kg to about 10 mg/kg. An indicated daily dosage in the larger mammal, including, but not limited to, humans, is in the range from about 3 mg to about 1500 mg, conveniently administered in a single dose or in divided doses, including, but not limited to, up to four times a day or in extended release form. Suitable unit dosage forms for oral administration include from about 1 to about 500 mg active ingredient. In one embodiment, the unit dosage is about 1 mg, about 5 mg, about, 10 mg, about 20 mg, about 50 mg, about 100 mg, about 200 mg, about 250 mg, about 400 mg, or about 500 mg. In another embodiment, the unit dosage is about 1 mg, about 5 mg, about, 10 mg, about 20 mg, about 25 mg, about 50 mg, about 75 mg, about 100 mg, about 150 mg, about 200 mg, about 250 mg, about 400 mg, or about 500 mg. The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

### Combination Treatments

Compound 1 or Compound 2 described herein, and compositions thereof, may also be used in combination with other therapeutic agents that are selected for their therapeutic value for the condition to be treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the clinician. The initial administration can be made according to established protocols recognized in the field, and then, based upon the observed effects, the dosage, modes of administration and times of administration can be modified by the clinician.

In certain instances, it may be appropriate to administer Compound 1 or Compound 2 described herein in combination with another therapeutic agent. By way of example only, if one of the side effects experienced by a patient upon receiving one of the compounds herein, such as Compound 1 or Compound 2, is nausea, then it may be appropriate to administer an anti-nausea agent in combination with the initial therapeutic agent. Or, by way of example only, the therapeutic effectiveness of one of the compounds described herein may be enhanced by administration of an adjuvant (i.e., by itself the adjuvant may have minimal therapeutic benefit, but in combination with another therapeutic agent, the overall therapeutic benefit to the patient is enhanced). Or, by way of example only, the benefit experienced by a patient may be increased by administering one of the compounds described herein with another therapeutic agent (which also includes a therapeutic regimen) that also has therapeutic benefit. In any case, regardless of the disease, disorder, or condition being treated, the overall benefit experienced by the patient may simply be additive of the two therapeutic agents or the patient may experience a synergistic benefit.

In some embodiments, Compound 1 or Compound 2 is administered in combination with a xanthine oxidase inhibitor. In some embodiments, Compound 1 or Compound 2 is administered in combination with a xanthine oxidase inhibitor, wherein the xanthine oxidase inhibitor is allopurinol, oxypurinol, febuxostat, topiroxostat, or inositol. In some embodiments, Compound 1 or Compound 2 is administered in combination with a xanthine oxidase inhibitor, wherein the xanthine oxidase inhibitor is allopurinol. In some embodiments, Compound 1 or Compound 2 is administered in combination with a xanthine oxidase inhibitor, wherein the xanthine oxidase inhibitor is oxypurinol. In some embodiments, Compound 1 or Compound 2 is administered in combination with a xanthine oxidase inhibitor, wherein the xanthine oxidase inhibitor is febuxostat. In some embodiments, Compound 1 or Compound 2 is administered in combination with a xanthine oxidase inhibitor, wherein the xanthine oxidase inhibitor is topiroxostat. In some embodiments, Compound 1 or Compound 2 is administered in combination with a xanthine oxidase inhibitor, wherein the xanthine oxidase inhibitor is ositol.

In some embodiments, Compound 1 or Compound 2 and the xanthine oxidase inhibitor are administered in combination in a single dosage form. In some embodiments, Compound 1 or Compound 2 and the xanthine oxidase inhibitor are administered in combination in separate dosage forms.

In some embodiments, Compound 1 or Compound 2 is administered in combination with an SGLT2 inhibitor. In some embodiments, Compound 1 or Compound 2 is administered in combination with an SGLT2 inhibitor, wherein the SGLT2 inhibitor is canagliflozin, dapagliflozin, empagliflozin, empagliflozin/linagliptin, empagliflozin/metformin, or dapagliflozin/metformin. In some embodiments, Compound 1 or Compound 2 is administered in combination with an SGLT2 inhibitor, wherein the SGLT2 inhibitor is canagliflozin. In some embodiments, Compound 1 or Compound 2 is administered in combination with an SGLT2 inhibitor, wherein the SGLT2 inhibitor is dapagliflozin. In some embodiments, Compound 1 or Compound 2 is administered in combination with an SGLT2 inhibitor, wherein the SGLT2 inhibitor is empagliflozin. In some embodiments, Compound 1 or Compound 2 is administered in combination with an SGLT2 inhibitor, wherein the SGLT2 inhibitor is empagliflozin/linagliptin. In some embodiments, Compound 1 or Compound 2 is administered in combination with an SGLT2 inhibitor, wherein the SGLT2 inhibitor is empagliflozin/metformin. In some embodiments, Compound 1 or Compound 2 is administered in combination with an SGLT2 inhibitor, wherein the SGLT2 inhibitor is dapagliflozin/metformin.

In some embodiments, Compound 1 or Compound 2 and the SGLT2 inhibitor are administered in combination in a single dosage form. In some embodiments, Compound 1 or Compound 2 and the SGLT2 inhibitor are administered in combination in separate dosage forms.

In some embodiments, Compound 1 or Compound 2 is administered in combination with a xanthine oxidase inhibitor and an SGLT2 inhibitor. In some embodiments, Compound 1 or Compound 2 is administered in combination with a xanthine oxidase inhibitor and an SGLT2 inhibitor, wherein the xanthine oxidase inhibitor is allopurinol, oxypurinol, febuxostat, topiroxostat, or inositol, and the SGLT2 inhibitor is canagliflozin, dapagliflozin, empagliflozin, empagliflozin/linagliptin, empagliflozin/metformin, or dapagliflozin/metformin.

In some embodiments, Compound 1 or Compound 2, the xanthine oxidase inhibitor, and the SGLT2 inhibitor are administered in combination in a single dosage form. In some embodiments, Compound 1 or Compound 2, the xanthine oxidase inhibitor, and the SGLT2 inhibitor are administered in combination in separate dosage forms.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the physician after evaluation of the disease being treated and the condition of the patient.

Therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is Compound 1 or Compound 2 described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents; the use of multiple therapeutic combinations are also envisioned.

The dosage regimen to treat, prevent, or ameliorate the condition(s) for which relief is sought, can be modified in accordance with a variety of factors. These factors include the disorder or condition from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from a few minutes to several hours, depending upon the properties of each pharmaceutical agent such as potency, solubility, bioavailability, plasma half-life and kinetic profile of the pharmaceutical agent. Circadian variation of the target molecule concentration may also determine the optimal dose interval.

In addition, the compounds described herein also may be used in combination with procedures that may provide additional or synergistic benefit to the patient. By way of example only, patients are expected to find therapeutic and/or prophylactic benefit in the methods described herein, wherein pharmaceutical composition of a compound disclosed herein and/or combinations with other therapeutics are combined with genetic testing to determine whether that individual is a carrier of a mutant gene that is correlated with certain diseases or conditions.

The compounds described herein and combination therapies can be administered before, during, or after the occurrence of a disease or condition, and the timing of administering the composition containing a compound can vary. Thus, for example, the compounds can be used as a prophylactic and can be administered continuously to subjects with a propensity to develop conditions or diseases in order to prevent the occurrence of the disease or condition. The initial administration can be via any route practical, such as, for example, an intravenous injection, a bolus injection, infusion over about 5 minutes to about 5 hours, a pill, a capsule, transdermal patch, buccal delivery, and the like, or combination thereof. A compound is preferably administered as soon as is practicable after the onset of a disease or condition is detected or suspected, and for a length of time necessary for the treatment of the disease or condition. The length of treatment can vary for each subject, and the length can be determined using specified criteria.

### EXAMPLES

List of Abbreviations

As used throughout the description of the invention, the following abbreviations, unless otherwise indicated, shall be understood to have the following meanings:
ACN or MeCN acetonitrile
Bn benzyl
BOC or Boc tert-butyl carbamate
t-Bu tert-butyl
Cy cyclohexyl
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DMF dimethylformamide
DMA N,N-dimethylacetamide
DMSO dimethylsulfoxide
eq or equiv equivalent(s)
Et ethyl
Et₂O diethyl ether
EtOH ethanol
EtOAc ethyl acetate
HPLC high performance liquid chromatography
Me methyl
MeOH methanol
MS mass spectroscopy
GC gas chromatography
h hour(s)
KF Karl Fischer
min minutes
MsOH methanesulfonic acid
NMR nuclear magnetic resonance
RP-HPLC reverse phase-high performance liquid chromatography
r.t. room temperature
TFA trifluoroacetic acid
THF tetrahydrofuran
TLC thin layer chromatography
V volumes

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: Preparation of (3,5-dibromo-4-hydroxyphenyl)(2-(1-hydroxyethyl)benzofuran-3-yl-4,5,6,7-d)methanone (Compound 1)

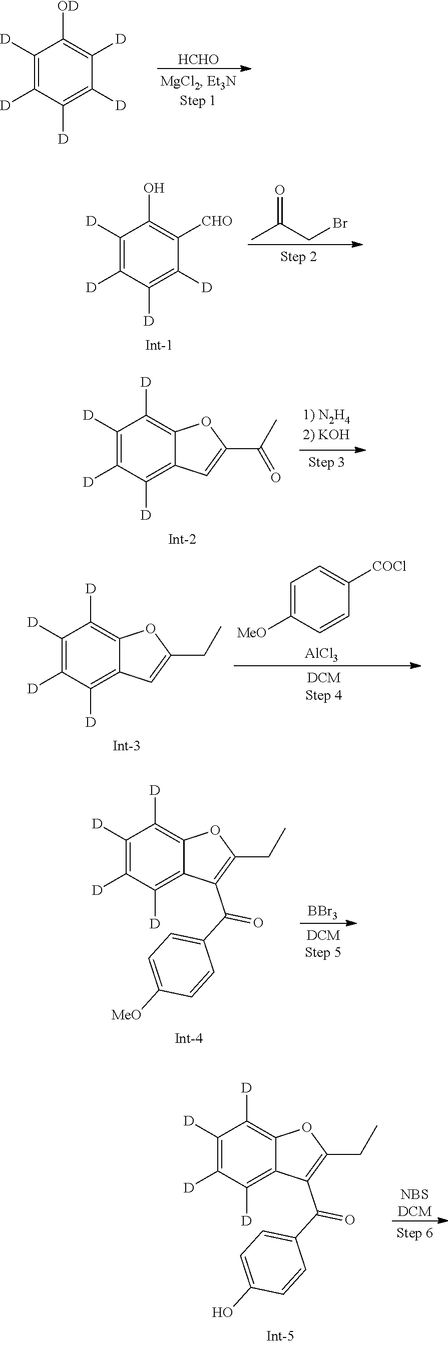

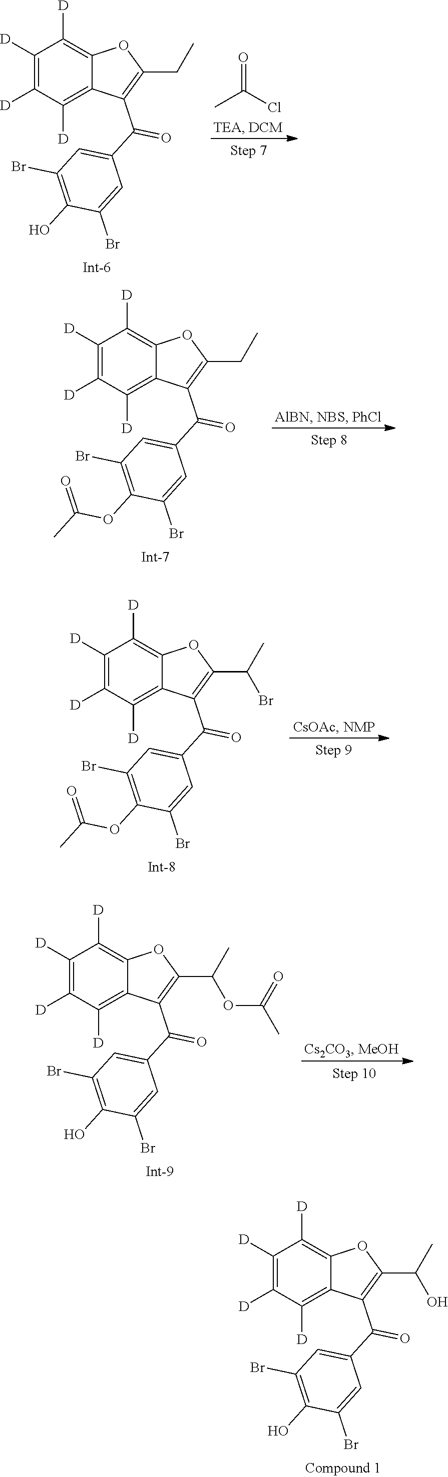

### Step 1: 2-Hydroxybenzaldehyde-3,4,5,6-d₄ (Int-1)

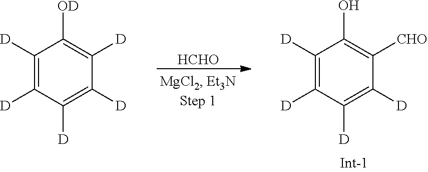

A solution of phen-d₆-ol (1.0 eq), magnesium chloride (1.5 eq), and triethylamine (3.7 eq) in ACN (10 V) was stirred at 20° C. for 0.5 h. Formaldehyde (8.0 eq) was added and the reaction mixture was heated at reflux for 3 h. The reaction mixture was cooled to room temperature and 10% HCl solution (10V) was added. The mixture was extracted with EtOAc (3×6V). The combined organic layers were washed with brine (6 V), dried with Na₂SO₄, and concentrated to give 2-hydroxybenzaldehyde-3,4,5,6-d₄ (Int-1) as a yellow oil.

### Step 2: 1-(Benzofuran-2-yl-4,5,6,7-d4)ethan-1-one (Int-2)

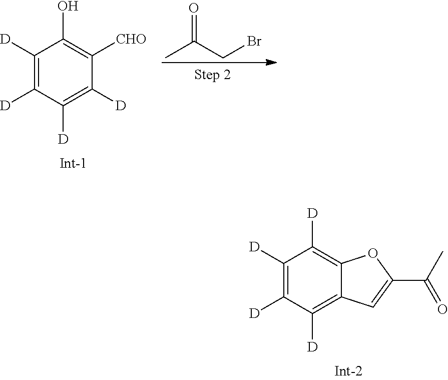

A solution of 2-hydroxybenzaldehyde-3,4,5,6-d₄ (Int-1) (1.0 eq), bromopropanone (1.0 eq), and potassium carbonate (3.0 eq) in acetone (14 V) was heated at reflux for 6 h. The reaction mixture was cooled to room temperature and filtered. The filtrate was concentrated and the crude product recrystallized (petroleum ether/EtOAc 10:1) to give 1-(benzofuran-2-yl-4,5,6,7-d4)ethan-1-one (Int-2) as a yellow solid.

### Step 3: 2-Ethylbenzofuran-4,5,6,7-d₄ (Int-3)

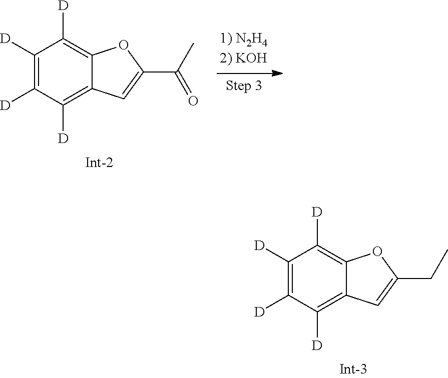 [SMILES: [2H]c1c([2H])c([2H])c2oc(C(C)=O)cc2c1[2H] ; [2H]c1c([2H])c([2H])c2oc(CC)cc2c1[2H]]

A solution of 1-(benzofuran-2-yl-4,5,6,7-d4)ethan-1-one (Int-2) (1.0 eq) in diethylene glycol (16 V) was heated at 120° C. N₂H₄·H₂O (2.0 eq) and water (1V) was added. The reaction mixture was heated at 180° C. for 10 min and then cooled to 120° C. KOH (2.0 eq) was added and the reaction mixture was heated at 120° C. for 6 h. The reaction mixture was cooled, poured into water, and extracted with EtOAc (20 V×3). The combined organic layers were washed with brine (20 V) and concentrated to give 2-ethylbenzofuran-4,5,6,7-d₄ (Int-3) as a colorless oil.

### Step 4: (2-Ethylbenzofuran-3-yl-4,5,6,7-d₄)(4-methoxyphenyl)methanone (Int-4)

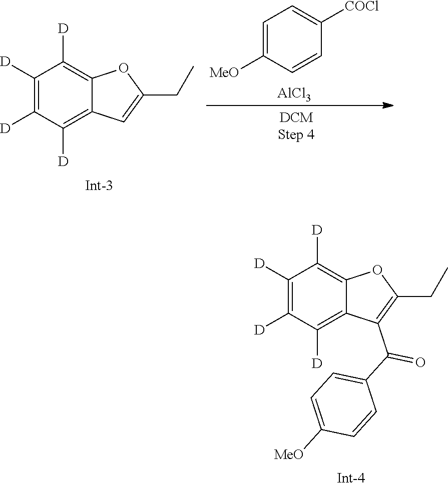

A solution of 2-ethylbenzofuran-4,5,6,7-d₄ (Int-3) (1.0 eq) and 4-methoxybenzoyl chloride (1.15 eq) in DCM (30 V) was cooled to 0° C. and charged with AlCl₃ (1.1 eq). The reaction mixture was stirred for 2 h at 0° C. D₂O (2 V) was added to the mixture dropwise at 5° C. and the mixture was stirred for 0.5 h. Water (8 V) was added. The organic layer was separated, washed with brine (10 V), dried with Na₂SO₄, and concentrated under vacuum at 40° C. to give (2-ethylbenzofuran-3-yl-4,5,6,7-d₄)(4-methoxyphenyl)methanone (Int-4) as a yellow solid. ¹H NMR (400 MHz, DMSO-d₆): δ 7.81-7.77 (dd, 2H), 7.12-7.08 (dd, 2H), 3.88 (s, 3H), 2.86-2.78 (q, 2H), 1.28-1.23 (t, 3H); LCMS: 285 [M+H]⁺.

### Step 5: (2-Ethylbenzofuran-3-yl-4,5,6,7-d)(4-hydroxyphenyl)methanone (Int-5)

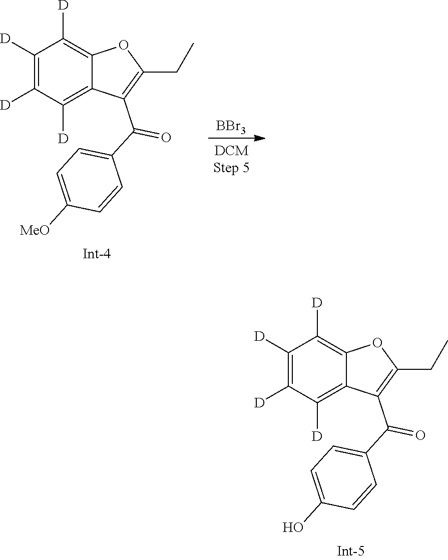

To a solution of (2-ethylbenzofuran-3-yl-4,5,6,7-d₄)(4-methoxyphenyl)methanone (Int-4) (1.0 eq) in DCM (10 V) at 0° C. was added BBr₃ (2.2 eq) dropwise at 0-5° C. The reaction mixture was warmed to room temperature and stirred for 14 h. Ice water (10 V) was added and the mixture was stirred for 0.5 h. The organic layer was separated, washed with brine (10 V), dried with Na₂SO₄, and concentrated under vacuum at 40° C. to give (2-ethylbenzofuran-3-yl-4,5,6,7-d₄)(4-hydroxyphenyl)methanone (Int-5) as a brown solid. ¹H NMR (400 MHz, DMSO-d₆): δ 10.47 (s, 1H), 7.71-7.68 (dd, 2H), 6.92-6.88 (dd, 2H), 2.84-2.78 (q, 2H), 1.28-1.24 (t, 3H); LCMS: 271 [M+H]⁺.

### Step 6: (3,5-Dibromo-4-hydroxyphenyl)(2-ethylbenzofuran-3-yl-4,5,6,7-4)methanone (Int-6)

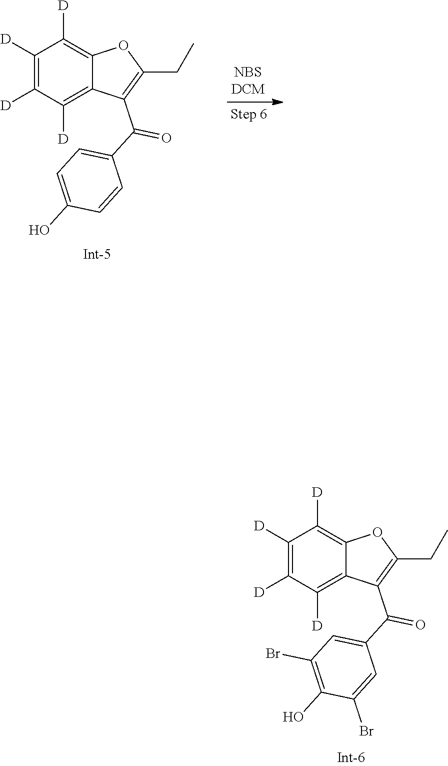 [SMILES: [2H]c1c([2H])c([2H])c2c(C(=O)c3ccc(O)cc3)c(CC)oc2c1[2H] ; [2H]c1c([2H])c([2H])c2c(C(=O)c3cc(Br)c(O)c(Br)c3)c(CC)oc2c1[2H]]

To a solution of (2-ethylbenzofuran-3-yl-4,5,6,7-d₄)(4-hydroxyphenyl)methanone (Int-5) (1.0 eq) in DCM (10 V) at 10° C. was added NBS (1.7 eq) dropwise at 0-5° C. The reaction mixture was warmed to 18° C. and stirred for 16 h. The reaction mixture was charged with additional NBS (0.14 eq) at 10° C. and stirred for 16 h at 18° C. The reaction mixture was charged with additional NBS (0.05 eq) at 10° C. and stirred for 3 h at 18° C. Water (15 V) was added and the mixture was stirred for 0.5 h. The organic layer was separated, washed with brine (15 V), dried with Na₂SO₄, and concentrated under vacuum at 40° C. to give a yellow solid. The yellow solid was slurried in EtOAc/n-heptane (1 V/10 V) at 60° C. for 2 h. The mixture was cooled to 10° C. and filtered to give (3,5-dibromo-4-hydroxyphenyl)(2-ethylbenzofuran-3-yl-4,5,6,7-d₄)methanone (Int-6) as a yellow solid. ¹H NMR (400 MHz, DMSO-d₆): δ 11.05 (s, 1H), 7.92 (s, 2H), 2.84-2.75 (q, 2H), 1.27-1.20 (t, 3H); LCMS: 429 [M+H]⁺.

### Step 7: 2,6-Dibromo-4-(2-ethylbenzofuran-3-carbonyl-4,5,6,7-4)phenyl acetate (Int-7)

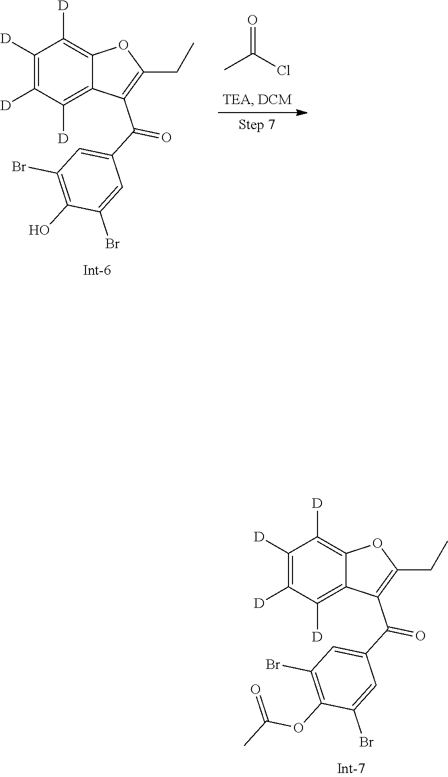

To a solution of (3,5-dibromo-4-hydroxyphenyl)(2-ethylbenzofuran-3-yl-4,5,6,7-d₄)methanone (Int-6) (1.0 eq) and triethylamine (2.5 eq) in DCM (10 V) at 0° C. was added acetyl chloride (2.0 eq) dropwise at 0-5° C. The reaction mixture was warmed to 15° C. and stirred for 2 h. Water (10 V) was added. The organic layer was separated, washed with brine (10 V), dried with Na₂SO₄, and concentrated under vacuum at 40° C. to give a crude solid. The crude solid was decolorized with activated charcoal (0.5 w/w) in EtOAc (10 V) at 50° C. for 1 h. The mixture was cooled to 30° C. and filtered with kieselguhr aid to remove the activated charcoal. The filtrate was concentrated under vacuum at 40° C. The residue was dissolved in i-PrOH (2 V) and heated at 60° C. for 1 h. The solution was cooled to 45° C., charged with seed crystals (0.5% w/w), and stirred for 1 h. The mixture was cooled to 25° C. and stirred for 16 h. The mixture was filtered and the solid dried to give 2,6-dibromo-4-(2-ethylbenzofuran-3-carbonyl-4,5,6,7-d₄)phenyl acetate (Int-7) as a yellow solid. ¹H NMR (400 MHz, DMSO-d₆): δ 8.08 (s, 2H), 2.81-2.74 (q, 2H), 2.44 (s, 3H), 1.27-1.22 (t, 3H); LCMS: 471 [M+H]⁺.

### Step 8: 2,6-dibromo-4-(2-(1-bromoethyl)benzofuran-3-carbonyl-4,5,6,7-d₄)phenyl acetate (Int-8)

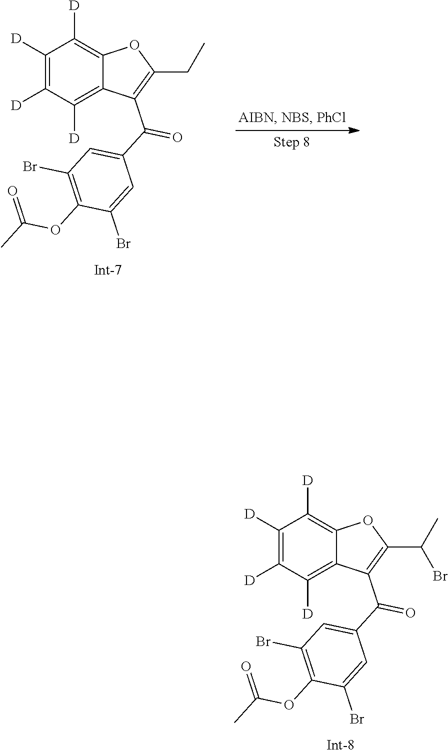 [SMILES: [2H]c1c([2H])c([2H])c2c(C(=O)c3cc(Br)c(OC(C)=O)c(Br)c3)c(CC)oc2c1[2H] ; [2H]c1c([2H])c([2H])c2c(C(=O)c3cc(Br)c(OC(C)=O)c(Br)c3)c(C(C)Br)oc2c1[2H]]

A mixture of 2,6-dibromo-4-(2-ethylbenzofuran-3-carbonyl-4,5,6,7-d₄)phenyl acetate (Int-7) (1.0 eq) NBS (1.1 eq) and AIBN (0.1 eq) in chlorobenzene (10 V) was heated at 55° C. for 6 h with stirring. The reaction mixture was cooled to 25° C., water (10 V) was added, and the mixture stirred for 1 h. The organic layer was separated, dried with Na₂SO₄, and concentrated to 1.5 to 2 V under vacuum. The solution was charged with heptane (5 V) and concentrated to 1.5 to 2 V under vacuum. This was repeated three times. The solution was charged with heptane (3 V), cooled to 5° C., and stirred for 4 h. The mixture was filtered and the solid washed with heptane (1 V×2), and dried to give 2,6-dibromo-4-(2-(1-bromoethyl)benzofuran-3-carbonyl-4,5,6,7-d₄)phenyl acetate (Int-8) as a yellow solid. ¹H NMR (400 MHz, DMSO-d₆): δ 8.11 (s, 2H), 5.47-5.40 (q, 1H), 2.46 (s, 3H), 2.05-2.03 (d, 3H); LCMS: 469 [M+H−HBr]⁺.

### Step 9: 1-(3-(3,5-Dibromo-4-hydroxybenzoyl)benzofuran-2-yl-4,5,6,7-d)ethyl acetate (Int-9)

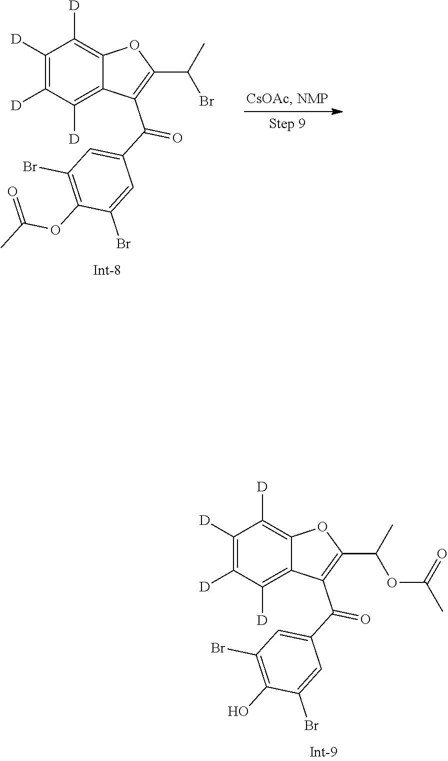 [SMILES: [2H]c1c([2H])c([2H])c2c(C(=O)c3cc(Br)c(OC(C)=O)c(Br)c3)c(C(C)Br)oc2c1[2H] ; [2H]c1c([2H])c([2H])c2c(C(=O)c3cc(Br)c(O)c(Br)c3)c(C(C)OC(C)=O)oc2c1[2H]]

A mixture of 2,6-dibromo-4-(2-(1-bromoethyl)benzofuran-3-carbonyl-4,5,6,7-d₄)phenyl acetate (Int-8) (1.0 eq) and CsOAc (5.0 eq) in N-methylpyrrolidine (8 V) was stirred at 25° C. for 12 h. The reaction mixture was filtered. To the filtrate was added water (15 V) and EtOAc (10 V). The pH of the resulting mixture was adjusted to 2-3 with 12 N HCl. The mixture was stirred for 1 h and then let stand for 0.5 h. The organic solution was collected and the aqueous solution extracted with EtOAc (10 V). The combined organic solution was washed with water (10 V×3), dried with Na₂SO₄, and concentrated under vacuum. The residue was purified by silica gel chromatography to give 1-(3-(3,5-dibromo-4-hydroxybenzoyl)benzofuran-2-yl-4,5,6,7-d₄)ethyl acetate (Int-9) as an off-white solid. ¹H NMR (400 MHz, DMSO-d₆): δ 7.93 (s, 2H), 5.88-5.87 (q, 1H), 1.99 (s, 3H), 1.63-1.61 (d, 3H); LCMS: 427 [M+H−CH₃CO₂H]⁺.

### Step 10: (3,5-Dibromo-4-hydroxyphenyl)(2-(1-hydroxyethyl)benzofuran-3-yl-4,5,6,7-d₄)methanone (Compound 1)

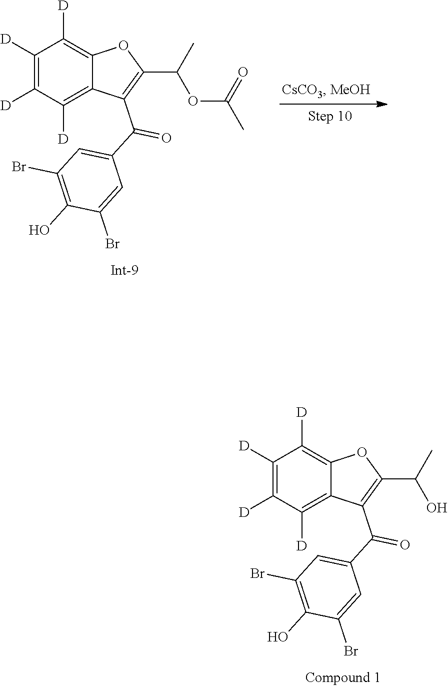 [SMILES: [2H]c1c([2H])c([2H])c2c(C(=O)c3cc(Br)c(O)c(Br)c3)c(C(C)OC(C)=O)oc2c1[2H] ; [2H]c1c([2H])c([2H])c2c(C(=O)c3cc(Br)c(O)c(Br)c3)c(C(C)O)oc2c1[2H]]

To a mixture of 1-(3-(3,5-dibromo-4-hydroxybenzoyl)benzofuran-2-yl-4,5,6,7-d₄)ethyl acetate (Int-9) (1.0 eq) in methanol (10 V) was added Cs₂CO₃ (3.0 eq). The reaction mixture was stirred at 28° C. for 12 h. Water (20 V) was added and the pH of the resulting mixture was adjusted to 2-3 with 12 N HCl. The mixture was stirred for 1 h. The mixture was filtered and the filter cake was washed with water (2 V×2). A solution of the filter cake, EtOAc (15 V) and 1 N HCl (5 V) was stirred for 1 h at 25° C. The organic solution was collected, dried with Na₂SO₄, and concentrated to 2 to 3 V under vacuum. The solution was heated at 50° C. for 1 h, charged with seed crystals (1% w/w), and heated at 50° C. for 2 h. n-Heptane (10 V) was added dropwise and the mixture was heated at 50° C. for 2 h. the mixture was cooled to 25° C. and stirred for 12 hours. The solid was collected by filtration and dried to give (3,5-dibromo-4-hydroxyphenyl)(2-(1-hydroxyethyl)benzofuran-3-yl-4,5,6,7-d₄)methanone (Compound 1) as an off-white solid. ¹H NMR (400 MHz, DMSO-d₆): δ 11.11 (bs, 1H), 7.95 (s, 1H), 5.60 (bs, 1H), 4.88-4.83 (q, 1H), 1.49-1.48 (d, 3H); LCMS: 427 [M+H−H₂O]⁺.

### Example 2: Preparation of (3,5-dibromo-4-hydroxyphenyl)(2-(1-hydroxyethyl)benzofuran-3-yl-4,5,6,7-d₄)methanone (Compound 2)

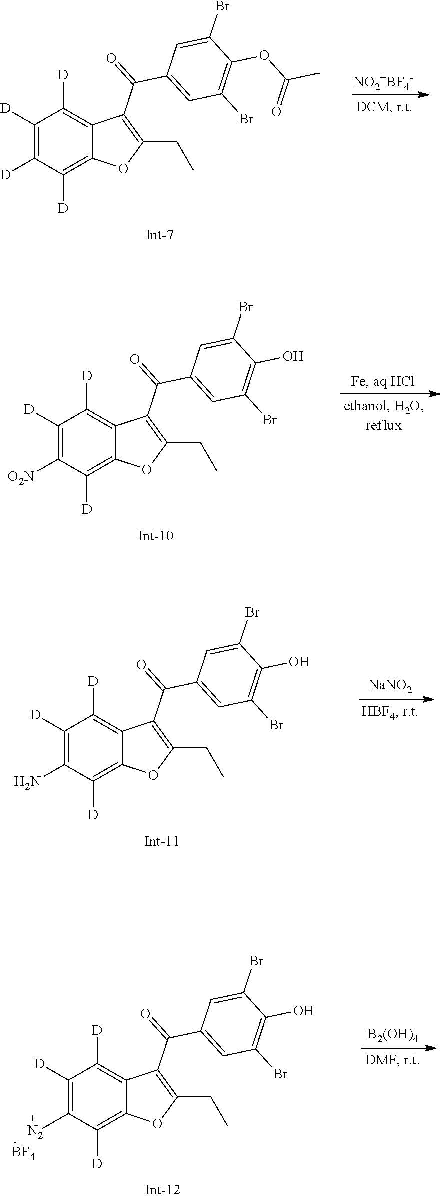

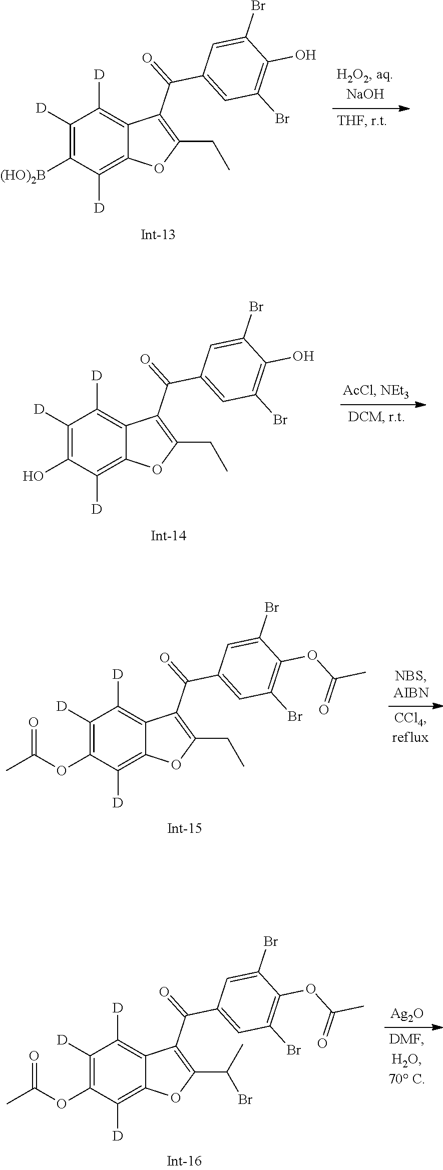

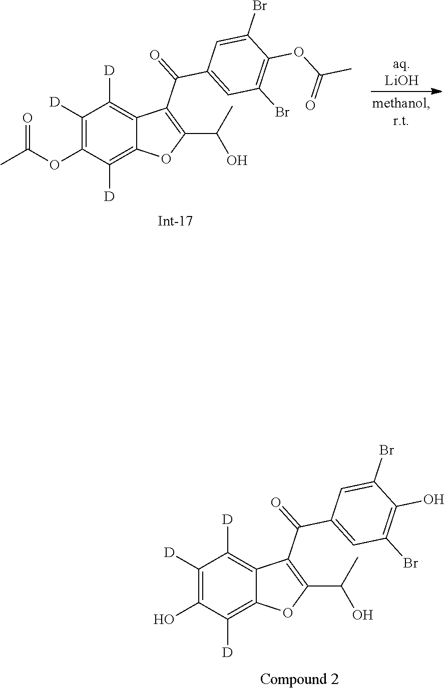 [SMILES: [2H]c1c(OC(C)=O)c([2H])c2oc(C(C)O)c(C(=O)c3cc(Br)c(OC(C)=O)c(Br)c3)c2c1[2H] ; "aq.LiOH" ; [2H]c1c(O)c([2H])c2oc(C(C)O)c(C(=O)c3cc(Br)c(O)c(Br)c3)c2c1[2H]]

### Step 1: (3,5-Dibromo-4-hydroxyphenyl)(2-ethyl-6-nitrobenzofuran-3-yl-4,5,7-d₃)methanone (Int-10)

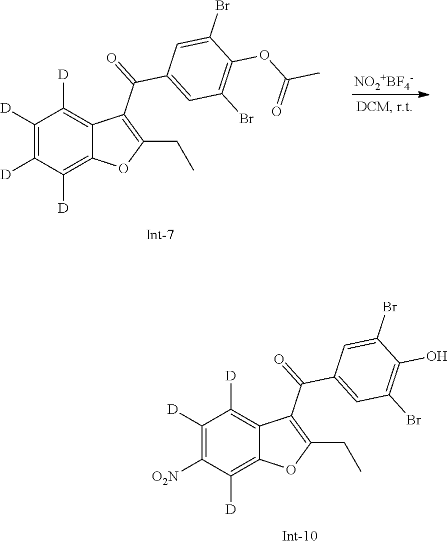

To a mixture of 2,6-dibromo-4-(2-ethylbenzofuran-3-carbonyl-4,5,6,7-d4)phenyl acetate (34 g, 72.3 mmol) (Int-7) in dichloromethane (250 mL) was added nitronium tetrafluoroborate (11.5 g, 86.8 mmol) slowly at 0° C. The reaction mixture was stirred at room temperature for overnight. The mixture was quenched with brine and extracted with dichloromethane. The organic phase was washed with brine, dried over sodium sulfate, and evaporated to dryness. The crude product was purified with column chromatography on silica gel to afford (3,5-dibromo hydroxyphenyl)(2-ethyl-6-nitrobenzofuran-3-yl-4,5,7-d₃)methanone (Int-10) (12.5 g, 37%) as a yellow solid. ¹H NMR (400 MHz, DMSO-d₆) δ 11.1 (bs, 1H), 7.98 (s, 2H), 2.87 (q, J=7.2 Hz, 2H), 1.3 (t, J=7.2 Hz, 3H).

### Step 2: (6-Amino-2-ethylbenzofuran-3-yl-4,5,7-d₃)(3,5-dibromo-4-hydroxyphenyl)methanone (Int-11)

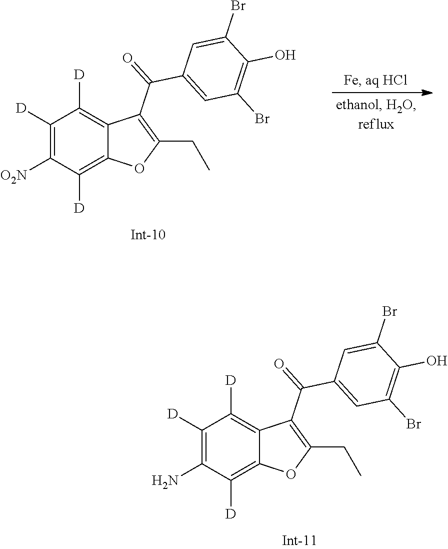 [SMILES: [2H]c1c([N+](=O)[O-])c([2H])c2oc(CC)c(C(=O)c3cc(Br)c(O)c(Br)c3)c2c1[2H] ; "Fe, aq HCl" ; [2H]c1c(N)c([2H])c2oc(CC)c(C(=O)c3cc(Br)c(O)c(Br)c3)c2c1[2H]]

A mixture of (3,5-dibromo-4-hydroxyphenyl)(2-ethyl-6-nitrobenzofuran-3-yl-4,5,7-d₃)methanone (Int-10) (12.5 g, 26.5 mol), iron powder (7.4 g, 132 mmol), conc. hydrochloride (1.2 mL, 13.3 mmol), ethanol (250 mL), and H₂O (50 mL) was heated to reflux for 2 hours. The mixture was filtered to remove the iron powder. The solution was evaporated to dryness. The residue was purified with column chromatography on silica gel to afford (6-amino-2-ethylbenzo-furan-3-yl-4,5,7-d₃)(3,5-dibromo-4-hydroxyphenyl)methanone (Int-11) (9.2 g, 79%) as a yellow solid. ESIMS (m/z): 441.9, 439.9, 437.9 (M+H)⁺.

### Step 3: 3-(3,5-Dibromo-4-hydroxybenzoyl)-2-ethylbenzofuran-6-diazonium-4,5,7-d₃ tetrafluoro-borate (Int-12)

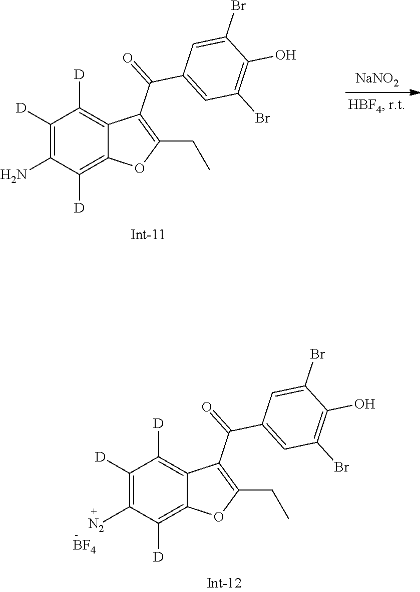

To a mixture of (6-amino-2-ethylbenzo-furan-3-yl-4,5,7-d₃)(3,5-dibromo-4-hydroxyphenyl)methanone (Int-11) (9.2 g, 20.8 mmol) and fluoroboric acid (4.2 g, 22.9 mmol) in H₂O (100 mL) was slowly added a solution of sodium nitrite (1.58 g, 22.9 mmol) in H₂O. The reaction mixture was stirred at room temperature overnight. The suspension obtained was then filtered in vacuum. The residue was washed with ice water and dried in vacuum to afford 3-(3,5-dibromo-4-hydroxybenzoyl)-2-ethylbenzofuran-6-diazonium-4,5,7-d₃ tetrafluoroborate (Int-12) (11 g, 98%) as a pink solid which was used for next steps without further purification. ESIMS (m/z): 455.9, 453.9, 451.9 (M+H)⁺.

### Step 4: (3-(3,5-Dibromo-4-hydroxybenzoyl)-2-ethylbenzofuran-6-yl-4,5,7-d₃)boronic acid (Int-13)

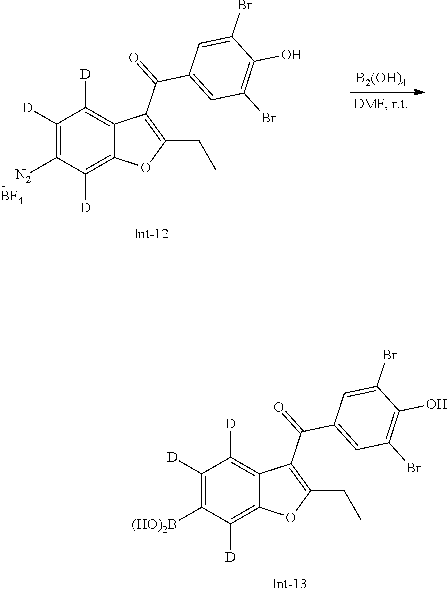

To a mixture of hypodiboric acid (3.65 g, 40.6 mmol) in DMF (160 mL) was added 3-(3,5-dibromo-4-hydroxybenzoyl)-2-ethylbenzofuran-6-diazonium-4,5,7-d₃ tetrafluoroborate (Int-12) (11 g, 20.3 mmol) at room temperature. The reaction mixture was stirred at the same temperature for 2 hours. The mixture was quenched with brine and extracted with ethyl acetate. The organic phase was washed with brine, dried over sodium sulfate, and evaporated to dryness. The residue was purified with column chromatography on silica gel to afford (3-(3,5-dibromo-4-hydroxybenzoyl)-2-ethylbenzofuran-6-yl-4,5,7-d₃)boronic acid (Int-13) (3.9 g, 41%) as a white solid. ESIMS (m/z): 473.9, 471.9, 469.9 (M+H)⁺.

### Step 5: (3,5-Dibromo-4-hydroxyphenyl)(2-ethyl-6-hydroxybenzofuran-3-yl-4,5,7-d₃)methanone (Int-14)

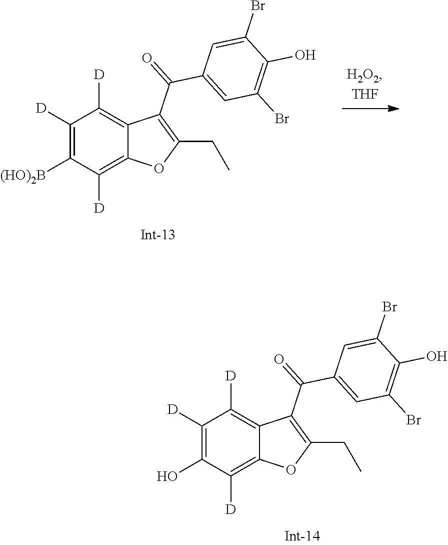

To a mixture of (3-(3,5-dibromo-4-hydroxybenzoyl)-2-ethylbenzofuran-6-yl-4,5,7-d₃)boronic (Int-13) (3.9 g, 8.28 mmol), and sodium hydroxide (662 mg, 16.6 mmol) in THF (60 mL) was added hydrogen peroxide (w/w 48%, 1.2 g, 16.6 mmol) dropwise at 0° C. The mixture was stirred at room temperature for 2 hours. It was quenched with 1M hydrochloride to pH 5-6, extracted with ethyl acetate. The organic phase was washed with brine, dried over sodium sulfate and evaporated to dryness. The residue was purified on silica gel with column chromatography to afford (3,5-dibromo-4-hydroxyphenyl)(2-ethyl-6-hydroxybenzofuran-3-yl-4,5,7-d₃)methanone (Int-14) (3.0 g, 83%) as a white solid. ¹H NMR (400 MHz, DMSO-d₆) δ 10.9 (bs, 1H), 9.68 (bs, 1H), 7.91 (s, 2H), 2.74 (q, J=7.6 Hz, 2H), 1.24 (t, J=7.6 Hz, 3H). ESIMS (m/z): 445.9, 443.9, 441.9 (M+H)⁺.

### Step 6: 4-(6-Acetoxy-2-ethylbenzofuran-3-carbonyl-4,5,7-d₃)-2,6-dibromophenyl acetate (Int-15)

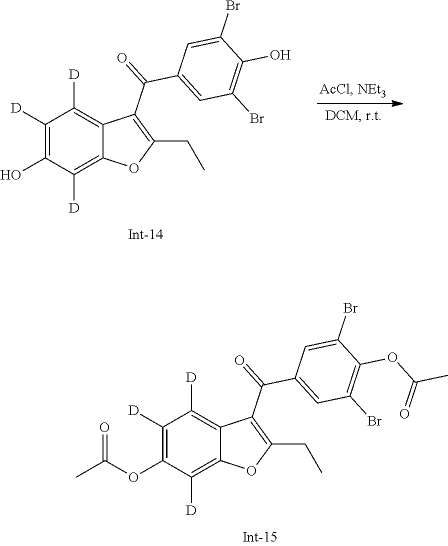

To a mixture of (3,5-dibromo-4-hydroxyphenyl)(2-ethyl-6-hydroxybenzofuran-3-yl-4,5,7-d₃)methanone (Int-14) (3.0 g. 6.78 mmol), and triethylamine (1.71 g, 17.0 mmol) in dichloromethane (60 mL) was added acetyl chloride (1.06 g, 13.6 mmol) dropwise at 0° C. The reaction mixture was stirred at room temperature for 2 h. The mixture was quenched with brine and extracted with ethyl acetate. The organic phase was washed with brine, dried over sodium sulfate, and evaporated to dryness. The residue was purified on silica gel with column chromatography to afford 4-(6-acetoxy-2-ethylbenzofuran-3-carbonyl-4,5,7-d₃)-2,6-dibromophenyl acetate (Int-15) (2.8 g, 78%) as a white solid.

### Step 7: 4-(6-Acetoxy-2-(1-bromoethyl)benzofuran-3-carbonyl-4,5,7-d₃)-2,6-dibromophenyl acetate (Int-16)

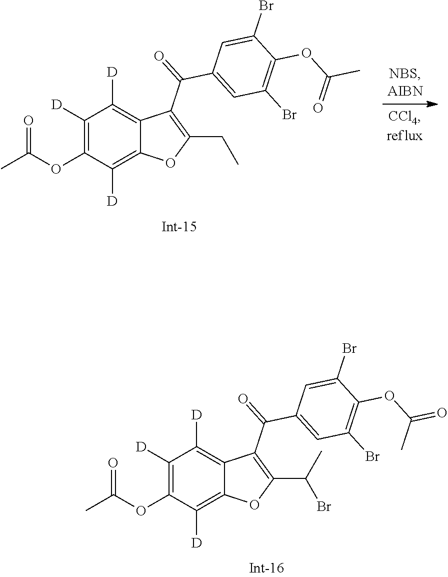

A mixture of 4-(6-acetoxy-2-ethylbenzofuran-3-carbonyl-4,5,7-d₃)-2,6-dibromophenyl acetate (Int-15) (2.8 g, 5.31 mmol), N-bromosuccinimide (945 mg, 5.31 mmol), and 2,2′-azobis(2-methylpropionitrile) (87 mg, 0.53 mmol) in perchloromethane (80 mL) was heated to reflux overnight. The mixture was evaporated to dryness. The crude was purified with column chromatography on silica gel to afford 4-(6-acetoxy-2-(1-bromoethyl)benzofuran-3-carbonyl-4,5,7-d₃)-2,6-dibromophenyl acetate (Int-16) (3.06 g, 95%) as a white solid.

### Step 8: 4-(6-Acetoxy-2-(1-hydroxyethyl)benzofuran-3-carbonyl-4,5,7-d₃)-2,6-dibromophenyl acetate (Int-17)

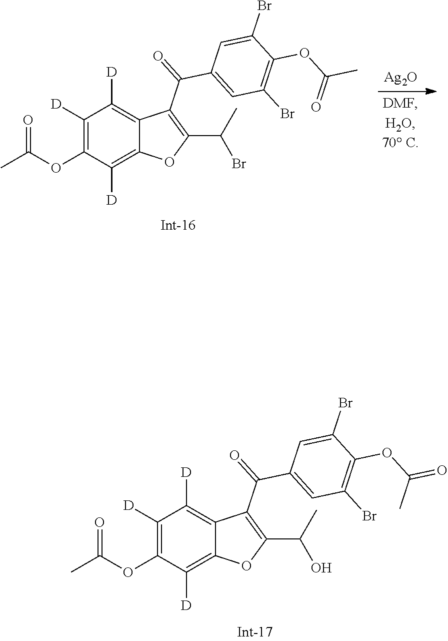

A mixture of 4-(6-acetoxy-2-(1-bromoethyl)benzofuran-3-carbonyl-4,5,7-d₃)-2,6-dibromophenyl acetate (Int-16) and silver(I) oxide (1.75 g, 7.57 mmol) in N,N-dimethylformamide (40 mL) and H₂O (8 mL) was heated to 70° C. for 6 hours. The mixture was filtered to remove the silver(I) oxide. The filtrate was quenched with brine and extracted with ethyl acetate. The organic phase was washed with brine, dried over sodium sulfate, and evaporated to dryness. The residue was purified on silica gel with column chromatography to afford 4-(6-acetoxy-2-(1-hydroxyethyl)benzofuran-3-carbonyl-4,5,7-d₃)-2,6-dibromophenyl acetate (Int-17) (2.3 g, 84%) as a white solid. ¹H NMR (400 MHz, DMSO-d₆) δ 11.7 (bs, 1H), 8.14 (bs, 1H), 5.65 (d, J=4.8 Hz, 1H), 4.80 (dd, J=6.8 Hz, 4.8 Hz, 1H), 2.45 (s, 3H), 2.33 (s, 3H), 1.48 (d, J=4.8 Hz, 3H). ESIMS (m/z): 527.9, 525.9, 523.9 (M+H)⁺.

### Step 9: (3,5-Dibromo-4-hydroxyphenyl)(6-hydroxy-2-(1-hydroxyethyl)benzofuran-3-yl-4,5,7-d₃)methanone (Compound 2)

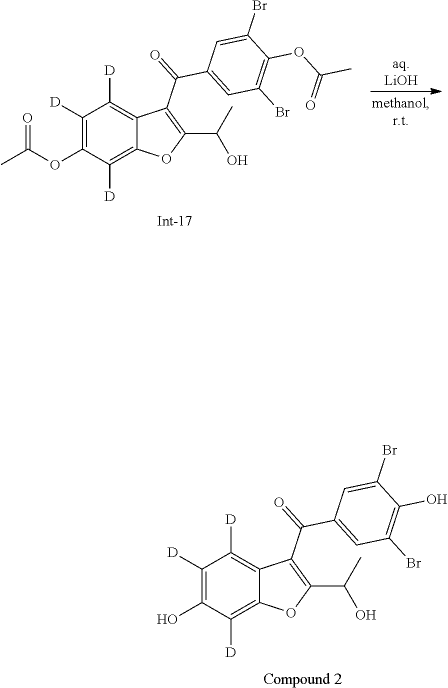 [SMILES: [2H]c1c(OC(C)=O)c([2H])c2oc(C(C)O)c(C(=O)c3cc(Br)c(OC(C)=O)c(Br)c3)c2c1[2H] ; "aq.LiOH" ; [2H]c1c(O)c([2H])c2oc(C(C)O)c(C(=O)c3cc(Br)c(O)c(Br)c3)c2c1[2H]]

A mixture of 4-(6-acetoxy-2-(1-hydroxyethyl)benzofuran-3-carbonyl-4,5,7-d₃)-2,6-dibromophenyl acetate (Int-17) (2.3 g, 4.24 mmol) and lithium hydroxide hydrate (427 mg, 10.2 mmol) in methanol (40 mL) and H₂O (5 mL) was stirred at room temperature for 2 hours. The mixture was poured into 30 mL of H₂O and evaporated in vacuum to remove methanol. The solution obtained was acidified to pH 5-6 with 1M hydrochloride and extracted with ethyl acetate. The organic phase was washed with brine, dried over sodium sulfate, and evaporated to dryness. The residue was purified with column chromatography on silica gel to afford (3,5-dibromo-4-hydroxyphenyl) (6-hydroxy-2-(1-hydroxyethyl)benzofuran-3-yl-4,5,7-d₃)methanone (Compound 2) (1.06 g, 55%) as a yellow solid. ¹H NMR (400 MHz, DMSO-d₆) δ 11.1 (bs, 1H), 9.75 (bs, 1H), 7.92 (s, 2H), 5.47 (bs, 1H), 4.77 (d, J=6.8 Hz, 1H), 1.45 (d, J=6.8 Hz, 3H). ESIMS (m/z): 443.9, 441.9, 439.9 (M+H)⁺.

### II. Biological Data

### Example 3: In Vitro Interaction Studies of Compound 1, Compound 2, Benzobromarone, and 1′-OH Benzbromarone with the Human URAT1 Uptake Transporter

Uptake experiments were performed using MDCKII cells stably expressing the human URAT1 uptake transporter. Cells were cultured at 37±1° C. in an atmosphere of 95:5 air:CO₂ and were plated onto standard 96-well tissue culture plates at the cell number described in Table 1.

TABLE 1

| | | Cell | | Incubation | |
|---|---|---|---|---|---|
| | Control | number/ | Culturing | prior to | |
| Transporter | cell line | well | medium | the assay | Buffer |
| human | Mock- | 1 × 10⁵ | DMEM | 24 h | HBSS w/o |
| URAT1 | transfected | | 4.5 g/L | | Cl⁻ |
| | MDCKII | | glucose | | (pH 7.4) |

DMEM: Dulbecco's Modified Eagle's Medium;
HBSS: Hank's balanced salt solution;
w/o: without

Before the experiment, the medium was removed and the cells were washed twice with 100 μL of HBSS without Cl⁻. Uptake experiments were carried out at 37±1° C. in 50 μL of HBSS without Cl⁻, pH 7.4 containing the probe substrate (20 μM uric acid) and the test article (TA) or solvent. The organic solvent concentration was equal in all wells, and did not exceed 1% (v/v).

Treatment groups are presented in Table 2.

TABLE 2

| Treatment groups in the 96-well plate format | No. of wells |
|---|---|
| Compound 1 in assay buffer (0.01, 0.04, 0.12, 0.37, | 3 per TA |
| 1.11, 3.33 and 10.0 μM) in transfected cells | concentration |
| Benzbromarone in assay buffer (0.01, 0.04, 0.12, | 3 per TA |
| 0.37, 1.11, 3.33 and 10.0 μM) in control cells | concentration |
| 1′-OH Benzbromarone in assay buffer (0.01, 0.02, | 3 per TA |
| 0.06, 0.19, 0.56, 1.67 and 5.0 μM) in control cells | concentration |
| 1% DMSO control in transfected cells | 3 |
| 1% DMSO control in control cells | 3 |
| Reference inhibitor in assay buffer with 1% DMSO in | 3 |
| transfected cells | |
| Reference inhibitor in assay buffer with 1% DMSO in | 3 |
| control cells | |

After the experiment, cells were washed twice with 100 μL of ice cold HBSS without Cl⁻ and lysed with 50 μL of 0.1 M NaOH. Radiolabeled probe substrate transport was determined by measuring an aliquot (35 μL) from each well for liquid scintillation counting.

Results: Test articles (Compound 1, Compound 2, benzbromarone, and 1′-OH benzbromarone) were soluble in HBSS buffer at all tested concentrations; the highest tested concentration being 10 μM. Compound 1 inhibited URAT1 mediated uric acid accumulation by 100% at a concentration of 10 μM with an IC₅₀=0.067 μM. Compound 2 inhibited URAT1 mediated uric acid accumulation with an IC₅₀=0.913 μM. Benzbromarone inhibited URAT1 mediated uric acid accumulation by 98% at a concentration of 10 μM with an IC₅₀=0.196 μM. 1′-OH benzbromarone inhibited URAT1 mediated uric acid accumulation by 94% at a concentration of 1.67 μM with an IC₅₀=0.050 μN.

### Example 4: A Renal Function Safety Pharmacology Study in Rats Following Single Oral Administration of Compound 1

Sixty-four Sprague Dawley Rats (32 per sex) were randomly assigned to four groups (8 animals per sex per group), and received a single oral gavage dose of 0.5% Methylcellulose (MC) in water (0 mg/kg of Compound 1) or Compound 1 at doses of 10, 30, 100 mg/kg in males and at doses of 5, 15, 50 mg/kg in females. Cageside and detailed clinical observations, body weights, water consumption, blood clinical chemistry, urine chemistry, and urinalysis were performed. Osmolality, osmolality gap, urine volume, water intake rate, fluid balance, urinary excretion, renal creatinine clearance, renal clearance, free water clearance, and fractional electrolyte clearance were calculated and evaluated. All animals were euthanized on Day 2 after scheduled observations. Blood samples for clinical chemistry analysis were collected from all the animals at 24 hours post dose prior to each scheduled euthanasia. Urine samples for clinical chemistry and urinalysis were collected from all the animals at pre-dose (˜24 hours) and 0-6 hours and 6-24 hours post-dose.

As shown in Table 3, Compound 1 reduced urine albumin-to-creatinine ratio (UACR) in rats. UACR is a key marker for chronic kidney disease and thus Compound 1 can potentially benefit CKD patients.

TABLE 3

| | | | | | 0-6 h post dose | | | | |
|---|---|---|---|---|---|---|---|---|---|
| | | Pre-dose (baseline) | | | | | | % UACR | |
| | Dose | UALB | UCRE | UACR | UALB | UCRE | UACR | change vs | % UACR |
| | (mg/kg) | (mg/L) | (g/L) | (mg/g) | (mg/L) | (g/L) | (mg/g) | baseline | change vs Ctrl |
| Male | 0 | 22.56 | 1.130 | 20.0 | 11.8 | 0.313 | 37.7 | 47% | |
| | 10 | 20.55 | 0.890 | 23.1 | 6.22 | 0.438 | 14.2 | −62% | −62% |
| | 30 | 27.51 | 1.217 | 22.6 | 8.73 | 0.563 | 15.5 | −46% | −59% |
| | 100 | 23.28 | 1.216 | 19.1 | 9.25 | 0.666 | 13.9 | −38% | −63% |
| Female | 0 | 26.64 | 1.037 | 25.7 | 16.38 | 0.382 | 42.9 | 40% | |
| | 5 | 26.81 | 0.958 | 28.0 | 33.71 | 0.328 | 102.7 | 73% | 140% |
| | 15 | 18.53 | 1.000 | 18.5 | 5.22 | 0.298 | 17.5 | −6% | −53% |
| | 50 | 34.54 | 1.203 | 28.7 | 9.58 | 0.409 | 23.4 | −23% | −38% |

| | | 6-24 h post dose | | | | |
|---|---|---|---|---|---|---|
| | | | | | % UACR | |
| | Dose | UALB | UCRE | UACR | change vs | % UACR |
| | (mg/kg) | (mg/L) | (g/L) | (mg/g) | baseline | change vs Ctrl |
| Male | 0 | 22.56 | 0.546 | 41.3 | 107% | |
| | 10 | 14.79 | 0.523 | 28.3 | 23% | −32% |
| | 30 | 12.38 | 0.418 | 29.6 | 31% | −28% |
| | 100 | 15.18 | 0.542 | 28.0 | 46% | −32% |
| Female | 0 | 23.95 | 0.704 | 34.0 | 32% | |
| | 5 | 24.2 | 0.600 | 40.3 | 44% | 19% |
| | 15 | 11.93 | 0.593 | 20.1 | 9% | −41% |
| | 50 | 21.15 | 0.790 | 26.8 | −7% | −21% |

### Example 5: In Vitro Metabolism of Compound 1 and Benzbromarone in Human Liver Microsomes

The incubation mixtures consisted of human liver microsomes (0.5 mg/mL protein), potassium phosphate buffer (100 mM, pH 7.4), 10 mM MgCl₂, and test article (2 μM of Compound 1) in a total volume of 0.5 mL. After pre-warming the mixture at 37° C. for 5 minutes, the reaction was initiated by the addition of 1 mM of NADPH and incubated for 60 minutes at 37° C. in a shaking water bath. The reaction was terminated by the addition of 0.5 mL ice-cold acetonitrile. Time 0 incubation was also performed by adding acetonitrile before the addition of microsomes to the incubation mixture as a reference sample for each species. Following vortex and centrifugation at 15,000 g for 3 minutes at room temperature, resulting supernatant was concentrated under nitrogen flow at 33° C., until approximately 0.5 mL of sample remained. The extract was then transferred to a 1.5-mL centrifuge vial for centrifugation at 15,000 g for 3 minutes. Finally, aliquots of the supernatant were transferred to HPLC vials for HPLC-MS analyses.

HPLC Analysis: Metabolic profiling of Compound 1 was conducted using a reverse phase C18 column and the conditions are summarized below:

| | |
|---|---|
| Analytical Column: | Supelco, Discovery C₁₈, 4.6 × 150 mm, |
| | 5 μm, |
| Column Temperature: | Ambient |
| Flow Rate | 0.9 mL/min |
| Injection Volume: | 10 μL |
| Injection Loop: | 100 μL |
| Autosampler Temperature | Ambient |
| Run Time: | 25 min |
| Mobile phase: | A = 10 mM ammonium acetate in water |
| | B = Acetonitrile |

MS Analysis: Mass spectral analyses were performed on an Agilent single quadruple mass spectrometer equipped with an Agilent 1100 HPLC system. The mass spectrometer was fitted with an electrospray ionization (ESI) source and operated in a negative scan mode between 400 and 500 amu:

| | | |
|---|---|---|
| Drying Gas Flow: | 12 | L/min |
| Nebulizer Pressure | 25 | psi |
| Drying Gas Temperature | 275° | C. |
| Capillary Voltage | 4000 | V |

| | |
|---|---|
| Polarity | Negative |
| Mass Scan Range | 400-500 |

Results: Compound 1 was stable in human microsomes following 60 minutes incubation with 82.5% of parent left. Compound 1 was converted to a carbonyl metabolite (M1) and a mono oxidative metabolite (M3) in human liver microsomes in the presence of NADPH.

| | % MS response | | | | | |
|---|---|---|---|---|---|---|
| | M1 | M2 | M3 | M4 | | |
| | (M − | (M + | (M + | (M + | Parent | |
| Species | 2) | 13) | 15) | 32) | (Compound 1) | Total |
| Human | 2.5 | 0.0 | 15.0 | 0.0 | 82.5 | 100.0 |

Metabolic profiling of benzbromarone was conducted using a similar method as described above. Following incubation, the majority of benzbromarone was metabolized to 6-OH benzbromarone, 1′-OH benzbromarone, and two di-hydroxy metabolites of benzbromarone, with benzbromarone accounted for 13.1% in the final mixture.

| | % MS Response | | | | | |
|---|---|---|---|---|---|---|
| | | | M3 | M4 | | |
| | M1 | M2 | (M + | (M + | Parent | |
| Species | (1′-OH) | (6-OH) | 32) | 32) | (benzbromarone) | Total |
| Human | 26.8 | 52.1 | 4.9 | 3.2 | 13.1 | 100.0 |

### Example 6: 4-Week Toxicity Study of Compound 1 in Sprague-Dawley Rats

Study Design: The study groups are shown in Table 4.

TABLE 4

| | | | | Main Study | | TK |
|---|---|---|---|---|---|---|
| | | Dose Level | Concen- | (No. of M/F) | | (No. of |
| Group | Treatment | (mg/kg/day) | tration | TS^{a} | RS^{b} | M/F) |
| 1 | Vehicle^{c} | 0 | 0 | 10/10 | 5/5 | 3/3 |
| 2 | Compound 1 | 2 | 0.2 | 10/10 | —/— | 6/6 |
| 3 | Compound 1 | 5 | 0.5 | 10/10 | —/— | 6/6 |
| 4 | Compound 1 | 15 | 1.5 | 10/10 | 5/5 | 6/6 |
| 5 | Compound 1 | 50 | 5 | 10/10 | 5/5 | 6/6 |

Main = Main Study animals; TK = TK animals; M = Male; F = Female; — = Not applicable.
^{a}Terminal sacrifice animals, to be sacrificed on Day 29.
^{b}Recovery sacrifice animals; not to be treated after Day 28 and to be sacrificed on Day 57.
^{c}0.5% MC in water

Animals were dosed with Compound 1 for 28 consecutive days with vehicle (0.5% (w/v) methylcellulose (MC) in water for injection) or Compound 1 in vehicle as outlined in Table 4 via oral gavage with a dosing volume of 10 mL/kg. On Day 29, surviving TS animals (all animals in study) were euthanized and subjected to a full gross necropsy, which included a macroscopic examination of the external surface of the body, all orifices, cranial cavity, external surface of the brain, the thoracic, abdominal and pelvic cavities and their viscera, cervical areas, carcass and genitalia. Organs were weighed as soon as possible from all main study animals at the scheduled necropsies. Tissues from Groups 1 and 5 animals at the terminal sacrifice, from animals found dead or moribund-sacrificed, and gross lesions from all animals, were embedded in paraffin, sectioned, stained with hematoxylin and eosin and examined microscopically by a board certified veterinary pathologist.

Results: No mortality was seen in any of the dosing groups (2, 5, 15, and 50 mg/kg/day) following 28 days of dosing with Compound 1. No abnormal clinical signs were observed. In addition, there was no effect on food consumption of body weight for any of the animals in the study. Liver weight increase in male at 50 mg/kg/day and female at ≥15 mg/kg/day; kidney weight increase in female at 50 mg/kg/day. Findings at high or mid-high dose resolved after 4 week recovery period. NOAEL level identified at 50 mg/kg (human equivalent to 500 mg).

### Example 7: 4-Week Toxicity Study of Compound 1 in Cynomolgus Monkeys

Study Design: The study groups are shown in Table 5.

TABLE 5

| | | | | No. of Animals | |
|---|---|---|---|---|---|
| | | Dose Level | | (M/F) | |
| Group | Treatment | (mg/kg/day) | Concentration | TS | RS |
| 1 | Vehicle | 0 | 0 | 4/4 | 2/2 |
| 2 | Compound 1 | 10 | 2 | 4/4 | —/— |
| 3 | Compound 1 | 30 | 6 | 4/4 | —/— |
| 4 | Compound 1 | 150 | 30 | 4/4 | 2/2 |

M = Male;
F = Female;
TS = Terminal sacrifice animals, to be sacrificed on Day 29.
RS = Recovery sacrifice animals; not to be treated after Day 28 and to be sacrificed on Day 57.

Animals were dosed with Compound 1 for 28 consecutive days with vehicle (0.5% (w/v) methylcellulose (MC) in water for injection) or Compound 1 in vehicle as outlined in Table 5 via oral intubation with a dosing volume of 5 mL/kg and rinsed by −5 mL drinking water. On Day 29, surviving TS animals (all animals in study) were euthanized and subjected to a full gross necropsy, which included a macroscopic examination of the external surface of the body, all orifices, cranial cavity, external surface of the brain, the thoracic, abdominal and pelvic cavities and their viscera, cervical areas, carcass and genitalia. Organs were weighed as soon as possible from all main study animals at the scheduled necropsies. Tissues from animals at the terminal sacrifice, from animals found dead or moribund-sacrificed, and gross lesions from all animals, were embedded in paraffin, sectioned, stained with hematoxylin and eosin and examined microscopically by a board certified veterinary pathologist.

Results: No mortality was seen in any of the dosing groups (10, 30, and 150 mg/kg/day) following 28 days of dosing with Compound 1. No abnormal clinical signs were observed. In addition, there was no effect on food consumption of body weight for any of the animals in the study. Liver weight trended higher in male at 150 mg/kg/day, but no statistical difference. No finding after 4 week recovery period. NOAEL level identified at 30 mg/kg (human equivalent to 600 mg).

### Example 8: Phase 1 Clinical Trial with Compound 1 (Single Dose)

Compound 1 was tested in a Phase 1, first-in-human, randomized, double-blind, placebo-controlled study conducted to evaluate the safety, tolerability, pharmacokinetics (PK), pharmacodynamics (PD), and preliminary food effects of single doses of Compound 1 in healthy adult males. The dose groups included Cohort 1 (15 mg or placebo, fasted, oral suspension), Cohort 2 (50 mg or placebo, fasted, suspension), Cohort 3 (100 mg or placebo, fasted, oral suspension), Cohort 4 (150 mg or placebo, fasted, oral suspension), Cohort 6 (50 mg or placebo, fed, oral suspension), and Cohort 7 (50 mg or placebo, fasted, oral capsule). A total of 35 subjects received a single dose Compound 1 (15 to 150 mg) in a fed or fasted state. An additional 8 subjects received placebo.

### Pharmacokinetics

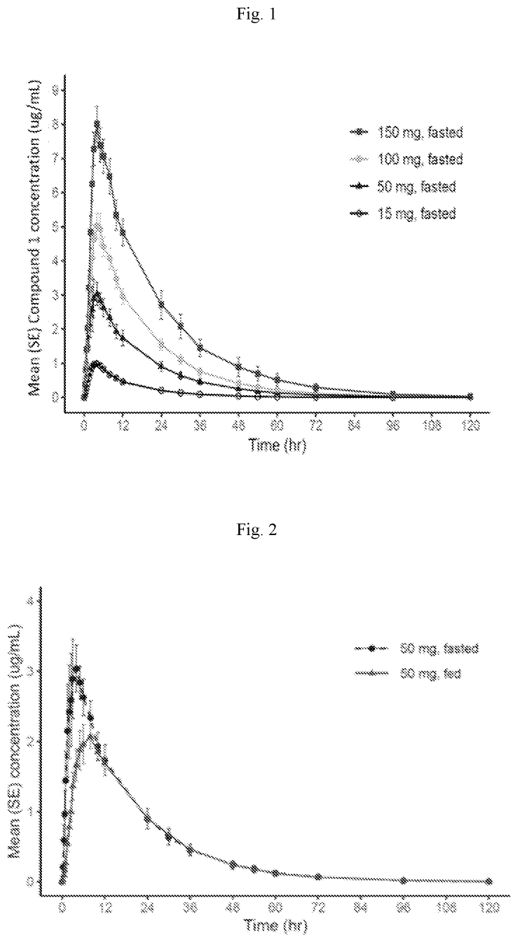

A Phase 1 study with Compound 1 in healthy adult males evaluated the plasma PK following single dose of Compound 1 or placebo. Compound 1 was administered as a racemic compound with the two enantiomers at a 1:1 ratio. Both the R-enantiomer and S-enantiomer of Compound 1 were monitored following single oral administration of racemic Compound 1. Rate of absorption of Compound 1 was moderate with median Tmax ranging from 3 to 5 hours post-dose under fasted conditions (FIG. 1 and Table 6). Absorption was delayed (median Tmax of 8 hours) when Compound 1 was administered in the fed state (FIG. 2 and Table 6). Plasma concentrations of Compound 1 declined with average terminal half-life values of approximately 10 to 13 hours (Table 6). Total plasma clearance of Compound 1 was approximately 1.0 L/hr with volume of distribution ranging from 14-18 L. In circulation, exposure of the S-enantiomer of Compound 1 was observed to be higher than the R-enantiomer of Compound 1. Ratio of S/R is approximately 1.7-1.8 for AUC_{inf} and 1.1 to 1.3 for Cmax (Table 7). The results are consistent with observation in animal testing and higher exposure of S-enantiomer was attributed to the conversion of the R-enantiomer in vivo.

TABLE 6

| Dose | Food | Cohort/ | ^{a}Tₘₐₓ | Cₘₐₓ | AUC₀₋₂₄ | AUC_{inf} | V_{z}/F | CL/F | t_{1/2} |
|---|---|---|---|---|---|---|---|---|---|
| (mg) | N | Form | (hr) | (μg/mL) | (μg · h/mL) | (μg · h/mL) | (L) | (L/hr) | (hr) |
| 15 | Fasted | C1/ | 3.50 | 1.06 | 12.1 | 15.0 | 14.1 | 1.02 | 9.69 |
| | N = 6 | Susp | (2.50-5.00) | (0.170) | (1.66) | (2.32) | (2.38) | (0.194) | (1.72) |
| 50 | Fasted | C2/ | 3.00 | 3.33 | 42.6 | 59.6 | 16.0 | 0.906 | 12.5 |
| | N = 5 | Susp | (3.00-5.00) | (0.834) | (11.3) | (18.5) | (3.90) | (0.279) | (1.84) |
| | Fed | C6/ | 8.00 | 2.21 | 34.0 | 51.2 | 16.3 | 1.03 | 11.7 |
| | N = 6 | Susp | (6.00-10.0) | (0.478) | (6.03) | (13.2) | (1.85) | (0.262) | (3.80) |
| | Fasted | C7/ | 1.75 | 3.97 | 41.9 | 51.3 | 14.9 | 1.01 | 10.5 |
| | N = 6 | Cap | (1.00-2.85) | (0.626) | (8.30) | (11.6) | (1.99) | (0.202) | (1.95) |
| 100 | Fasted | C3/ | 4.00 | 5.51 | 71.0 | 99.7 | 18.6 | 1.04 | 12.5 |
| | N = 6 | Susp | (2.00-6.03) | (1.05) | (11.5) | (20.9) | (4.82) | (0.224) | (2.63) |
| 150 | Fasted | C4/ | 4.00 | 8.33 | 114 | 173 | 15.5 | 0.944 | 12.0 |
| | N = 6 | Susp | (3.00-5.00) | (1.27) | (21.3) | (56.3) | (2.84) | (0.302) | (2.90) |

Abbreviations: AUC_{inf}, area under the concentration-time curve from 0 to infinity; CL/F, total body clearance corrected for bioavailability; Cap, IR capsule; Cₘₐₓ, maximum concentration; Form, formulation; N, number of subjects; Susp, suspension; t½, apparent terminal half-life; Tₘₐₓ, time to reach maximum concentration; V_{z}/F, volume of distribution corrected for bioavailability.
^{a}Tₘₐₓ values are represented by median (range).

TABLE 7

| Dose | Food | Cohort/ | Cₘₐₓ (μg/mL) | | | AUC₀₋₂₄ (μg · h/mL) | | | AUC_{inf} (μg · h/mL) | | | Ratio (−S/−R) | | |
|---|---|---|---|---|---|---|---|---|---|---|---|---|---|---|
| (mg) | N | Form | R− | S− | Total | R− | S− | Total | R− | S− | Total | Cₘₐₓ | AUC₀₋₂₄ | AUC_{inf} |
| 15 | Fasted | C1/ | 0.465 | 0.593 | 1.06 | 4.80 | 7.28 | 12.1 | 5.54 | 9.55 | 15.0 | 1.28 | 1.52 | 1.72 |
| | N = 6 | Susp | (0.0780) | (0.0924) | (0.170) | (0.687) | (1.01) | (1.66) | (0.848) | (1.57) | (2.32) | (0.0411) | (0.0934) | (0.163) |
| 50 | Fasted | C2/ | 1.53 | 1.80 | 3.33 | 17.5 | 25.2 | 42.6 | 21.8 | 38.0 | 59.6 | 1.18 | 1.45 | 1.75 |
| | N = 5 | Susp | (0.401) | (0.434) | (0.834) | (4.89) | (6.46) | (11.3) | (6.87) | (11.6) | (18.5) | (0.0392) | (0.0731) | (0.111) |
| | Fed | C6/ | 0.974 | 1.24 | 2.21 | 13.9 | 20.1 | 34.0 | 18.0 | 33.2 | 51.2 | 1.28 | 1.44 | 1.82 |
| | N = 6 | Susp | (0.234) | (0.253) | (0.478) | (2.43) | (3.64) | (6.03) | (3.46) | (9.97) | (13.2) | (0.0813) | (0.0649) | (0.250) |
| | Fasted | C7/ | 1.87 | 2.11 | 3.97 | 17.1 | 24.8 | 41.9 | 19.3 | 32.0 | 51.3 | 1.13 | 1.46 | 1.66 |
| | N = 5 | Cap | (0.316) | (0.313) | (0.626) | (3.76) | (4.59) | (8.30) | (4.60) | (7.05) | (11.6) | (0.0499) | (0.0827) | (0.109) |
| 100 | Fasted | C3/ | 2.58 | 2.93 | 5.51 | 29.4 | 41.7 | 71.0 | 36.5 | 63.2 | 99.7 | 1.14 | 1.43 | 1.73 |
| | N = 6 | Susp | (0.524) | (0.534) | (1.05) | (5.25) | (6.46) | (11.5) | (7.62) | (13.5) | (20.9) | (0.0607) | (0.0863) | (0.108) |
| 150 | Fasted | C4/ | 4.02 | 4.33 | 8.33 | 48.1 | 65.9 | 114 | 62.7 | 110 | 173 | 1.08 | 1.37 | 1.73 |
| | N = 6 | Susp | (0.678) | (0.599) | (1.27) | (9.11) | (12.2) | (21.3) | (17.6) | (38.8) | (56.3) | (0.0400) | (0.0308) | (0.112) |

Cap: capsule; Form: formulation; Susp: suspension.

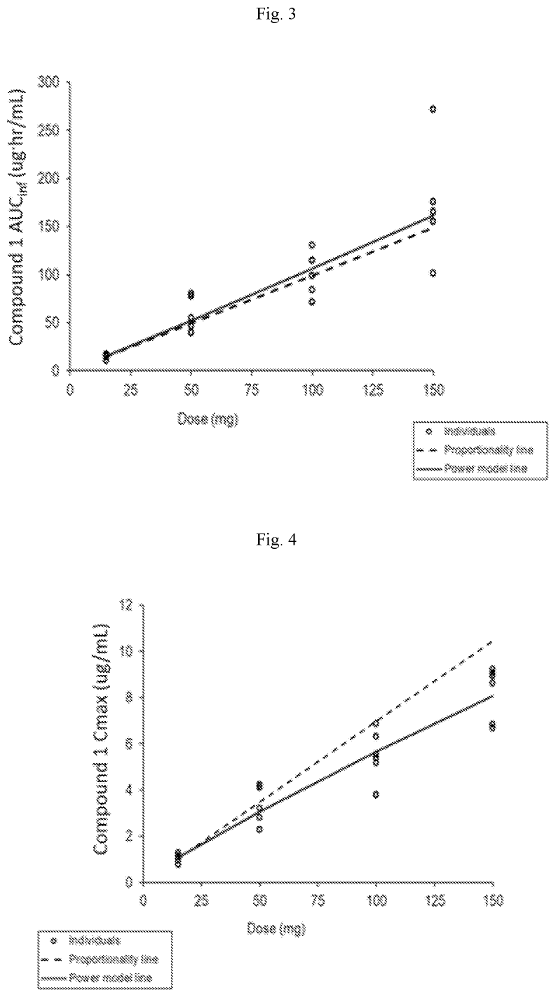

Statistical assessment of dose proportionality showed that plasma exposures AUC (AUCs) of Compound 1 displayed dose proportional increases (power model exponent=1.0, CI95% within 0.8-1.25 limit) in the 15 to 150 mg dose range under fasted conditions while Cmax exhibited slightly less than dose proportionality increase (power model exponent=0.88, CI95% is outside 0.8-1.25 limit) (Table 8). FIG. 3 and FIG. 4 shows increase of AUC (FIG. 3) and Cmax (FIG. 4) with dose and their agreement or deviation from dose proportionality line.

TABLE 8

| | | Power | | |
|---|---|---|---|---|
| Dependent | Intercept | exponent | Lower_CI95% | Upper_CI95% |
| Cₘₐₓ | 0.0966 | 0.884 | 0.790 | 0.978 |
| (μg/mL) | | | | |
| AUC_{inf} | 0.944 | 1.03 | 0.902 | 1.15 |
| (μg · h/mL) | | | | |

Power model is expressed as Cₘₐₓ or AUC = a × dose^{b}, where a is intercept and b is power exponent. If b is close to unity of 1 with CI95% falling within 0.8 and 1.25, dose proportionality is concluded.

Food effect was assessed for the 50 mg dose level. Group with Compound 1 administered in the fed state (high-fat, high-calorie meal) showed slower absorption (Tmax delayed from 3-5 hours in the fasted state to 8 hours post-dose) and lower exposure than when Compound 1 was administered under fasted conditions (Tables 6 and 9). The impact of dosing under fed condition was noted as having a more significant effect on Cmax than AUC (Table 9 and FIG. 2). In the fed state, Cmax was ˜33% while AUC decreased approximately 13% with food.

TABLE 9

| | | | | | | Geometric Mean | |
|---|---|---|---|---|---|---|---|
| | | | | Geometric Least Squares Mean | | Ratio, % (90% CI) | |
| Dose | N | | Variable | Fed | Fasted | (Fed/Fasted) | |
| 50 mg | 5 | (fasted) | Cₘₐₓ | 2.17 | 3.25 | 66.7 | (51.1-87.2) |
| | 6 | (fed) | AUCₗₐₛₜ | 49.6 | 57.1 | 86.9 | (63.5-119) |

| | | | | |
|---|---|---|---|---|
| AUC_{inf} | 49.9 | 57.4 | 86.9 | (63.5-119) |

### Pharmacodynamics

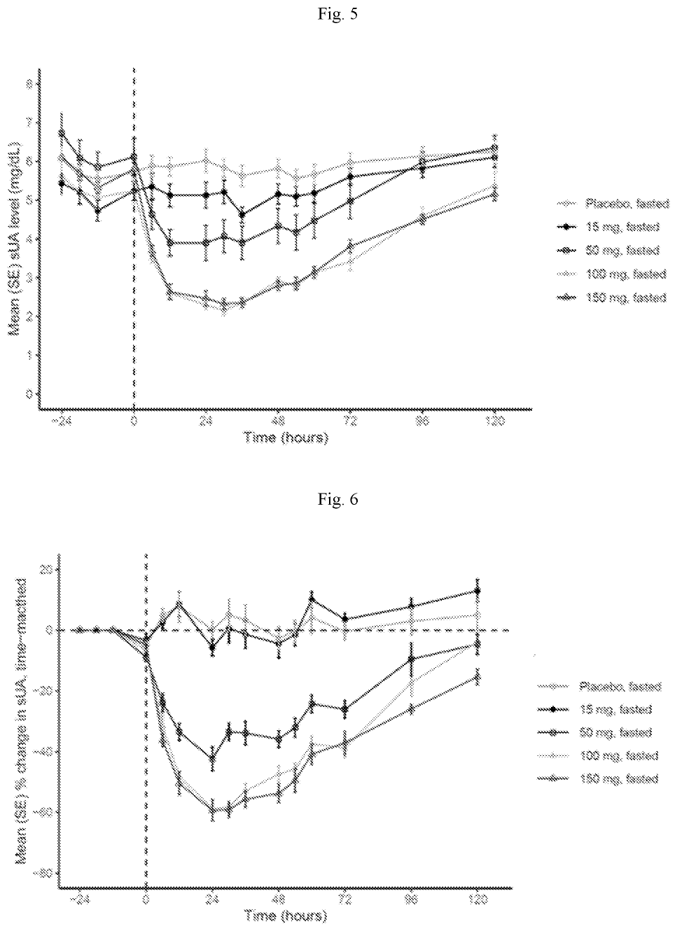

Upon administration of a single oral dose of Compound 1 at 15, 50, 100, and 150 mg under the fasted condition, the mean sUA levels showed dose-dependent decreases (Table 10 and FIG. 5) at 50 mg or higher dosing when compared to placebo group. Decrease in sUA appeared to reach a plateau at 100 mg or lower. At 50 mg or higher doses, the onset of sUA lowering effect was observed at 6 hours post-dose and achieved nadir at 24-36 hours post-dose. sUA remained lower than baseline levels for up to 72 hours post-dose, indicating a sustained sUA lowering effect and a significantly longer PD effect than the PK half-life of Compound 1. Under fasted conditions, administration of 15, 50, 100 and 150 mg Compound 1 produced approximately −6%, −42%, −59%, and −59% mean reduction in sUA levels at 24 hours post-dose, respectively, compared with an approximately 0.2% increase in the pooled placebo group (Table 11 and FIG. 6). The reduction in sUA levels at 24 hours post-dose is partly attributed to the formation of Compound 1 metabolite, 6-OH Compound 1.

TABLE 10
Seram uric acid (mg/dL), Time (hours)

| Treatment | | −24 | −18 | −12 | 0 | 6 | 12 | 24 | 30 | 36 | 48 | 54 | 60 | 72 | 96 | 120 |
|---|---|---|---|---|---|---|---|---|---|---|---|---|---|---|---|---|
| placebo, fasted | Mean | 6.1 | 5.6 | 5.5 | 5.7 | 5.9 | 5.9 | 6.0 | 5.8 | 5.6 | 5.8 | 5.6 | 5.7 | 6.0 | 6.1 | 6.3 |
| (N = 10) | SD | 1.1 | 1.0 | 1.1 | 0.9 | 0.8 | 0.7 | 0.9 | 0.7 | 0.8 | 0.8 | 0.7 | 0.8 | 0.8 | 0.8 | 1.0 |
| placebo, fed | Mean | 4.8 | 4.6 | 4.5 | 4.9 | 4.5 | 4.4 | 5.0 | 4.8 | 4.7 | 5.0 | 5.0 | 4.8 | 5.1 | 5.3 | 5.0 |
| (N = 2) | SD | 0.4 | 0.4 | 0.4 | 0.2 | 0.4 | 0.5 | 0.4 | 0.4 | 0.2 | 0.1 | 0.6 | 0.4 | 0.1 | 0.6 | 0.2 |
| 15 mg, fasted | Mean | 5.4 | 5.2 | 4.7 | 5.2 | 5.4 | 5.1 | 5.1 | 5.2 | 4.6 | 5.2 | 5.1 | 5.2 | 5.6 | 5.8 | 6.1 |
| (N = 6) | SD | 0.7 | 0.7 | 0.7 | 0.6 | 0.8 | 0.7 | 0.8 | 0.7 | 0.5 | 0.6 | 0.6 | 0.6 | 0.6 | 0.6 | 0.6 |
| 50 mg, fasted | Mean | 6.7 | 6.1 | 5.9 | 6.1 | 4.6 | 3.9 | 3.9 | 4.1 | 3.9 | 4.3 | 4.2 | 4.5 | 5.0 | 6.0 | 6.4 |
| (N = 5) | SD | 1.2 | 1.0 | 0.9 | 1.1 | 0.9 | 0.8 | 1.0 | 0.9 | 1.0 | 1.0 | 1.0 | 1.0 | 1.0 | 0.6 | 0.7 |
| 50 mg, fed | Mean | 6.5 | 6.0 | 6.2 | 6.4 | 4.8 | 3.9 | 3.8 | 3.6 | 3.9 | 4.4 | 4.2 | 4.3 | 5.0 | 5.8 | 6.1 |
| (N = 6) | SD | 1.1 | 1.0 | 1.0 | 0.9 | 1.0 | 1.0 | 1.0 | 1.0 | 1.0 | 1.0 | 0.9 | 1.0 | 1.0 | 1.3 | 1.2 |
| 50 mg, fasted | Mean | 6.0 | 5.5 | 5.5 | 5.4 | 4.0 | 3.4 | 3.7 | 3.4 | 3.6 | 4.1 | 3.9 | 4.3 | 4.9 | 5.3 | 5.2 |
| Capsule, (N = 6) | SD | 0.8 | 0.8 | 0.7 | 0.7 | 0.6 | 0.6 | 0.5 | 0.6 | 0.8 | 0.6 | 0.6 | 0.5 | 0.6 | 0.7 | 0.5 |
| 100 mg, fasted | Mean | 5.6 | 5.2 | 5.1 | 5.2 | 3.5 | 2.6 | 2.3 | 2.2 | 2.4 | 2.9 | 2.8 | 3.1 | 3.4 | 4.6 | 5.4 |
| (N = 6) | SD | 0.9 | 0.8 | 0.7 | 0.8 | 0.3 | 0.3 | 0.3 | 0.3 | 0.2 | 0.2 | 0.3 | 0.3 | 0.5 | 0.6 | 0.9 |
| 150 mg, fasted | Mean | 6.1 | 5.7 | 5.3 | 5.8 | 3.7 | 2.6 | 2.5 | 2.3 | 2.4 | 2.8 | 2.9 | 3.1 | 3.8 | 4.5 | 5.2 |
| (N = 6) | SD | 0.5 | 0.5 | 0.3 | 0.4 | 0.3 | 0.5 | 0.5 | 0.3 | 0.3 | 0.3 | 0.4 | 0.4 | 0.4 | 0.3 | 0.4 |

TABLE 11
Percent change in serum uric acid, Time (hours)

| Treatment | Time | −24 | −18 | −12 | 0 | 6 | 12 | 24 | 30 | 36 | 48 | 54 | 60 | 72 | 96 | 120 |
|---|---|---|---|---|---|---|---|---|---|---|---|---|---|---|---|---|
| placebo, fasted | Mean | 0 | 0 | 0 | −5.3 | 4.9 | 7.8 | 0.2 | 5.0 | 3.3 | −2.7 | −0.3 | 4.1 | −0.4 | 3.0 | 5.0 |
| (N = 10) | SD | 0 | 0 | 0 | 3.3 | 7.0 | 15.9 | 8.6 | 16.4 | 15.9 | 11.8 | 11.2 | 16.4 | 8.8 | 14.3 | 16.7 |
| placebo, fed | Mean | 0 | 0 | 0 | 1.9 | −3.6 | −2.0 | 3.5 | 3.6 | 5.8 | 3.7 | 7.1 | 7.6 | 7.2 | 11.3 | 5.7 |
| (N = 2) | SD | 0 | 0 | 0 | 2.6 | 0.3 | 2.8 | 0.3 | 0.3 | 3.1 | 5.2 | 4.6 | 0.6 | 5.5 | 20.7 | 12.8 |
| 15 mg, fasted | Mean | 0 | 0 | 0 | −3.4 | 2.7 | 8.7 | −5.8 | 0.7 | −1.4 | −4.4 | −1.5 | 10.0 | 3.6 | 7.8 | 13.0 |
| (N = 6) | SD | 0 | 0 | 0 | 2.7 | 5.1 | 9.7 | 6.5 | 11.8 | 11.2 | 11.5 | 8.9 | 6.6 | 5.2 | 6.6 | 9.0 |
| 50 mg, fasted | Mean | 0 | 0 | 0 | −8.9 | −23.8 | −33.6 | −42.4 | −33.5 | −33.9 | −35.8 | −32.1 | −24.2 | −26.1 | −9.6 | −4.6 |
| (N = 5) | SD | 0 | 0 | 0 | 2.9 | 6.7 | 6.2 | 8.8 | 6.5 | 8.9 | 6.1 | 7.0 | 6.5 | 6.5 | 12.0 | 7.3 |
| 50 mg, fed | Mean | 0 | 0 | 0 | −1.0 | −20.3 | −37.7 | −41.9 | −40.1 | −37.4 | −32.6 | −29.8 | −31.3 | −23.3 | −10.9 | −5.8 |
| (N = 6) | SD | 0 | 0 | 0 | 2.7 | 4.1 | 7.4 | 8.1 | 9.4 | 7.4 | 4.8 | 6.3 | 6.5 | 2.5 | 10.4 | 7.0 |
| 50 mg, fasted | Mean | 0 | 0 | 0 | −9.6 | −26.9 | −37.5 | −38.3 | −36.9 | −34.1 | −32.4 | −28.1 | −21.0 | −17.8 | −11.2 | −12.5 |
| Capsule, (N = 6) | SD | 0 | 0 | 0 | 4.7 | 6.1 | 8.0 | 8.3 | 9.7 | 11.0 | 8.1 | 9.3 | 7.3 | 6.7 | 10.8 | 8.0 |
| 100 mg, fasted | Mean | 0 | 0 | 0 | −6.6 | −31.6 | −48.2 | −58.8 | −58.2 | −52.9 | −47.4 | −45.7 | −37.5 | −38.7 | −17.3 | −3.8 |
| (N = 6) | SD | 0 | 0 | 0 | 7.2 | 9.5 | 4.4 | 4.0 | 4.9 | 6.0 | 6.6 | 4.6 | 6.1 | 7.9 | 11.0 | 10.8 |
| 150 mg, fasted | Mean | 0 | 0 | 0 | −4.6 | −36.2 | −50.4 | −59.3 | −59.1 | −55.6 | −53.8 | −49.5 | −40.8 | −37.1 | −25.9 | −15.4 |
| (N = 6) | SD | 0 | 0 | 0 | 8.5 | 4.7 | 9.1 | 8.5 | 6.1 | 6.6 | 7.1 | 9.5 | 8.4 | 9.4 | 3.8 | 6.0 |

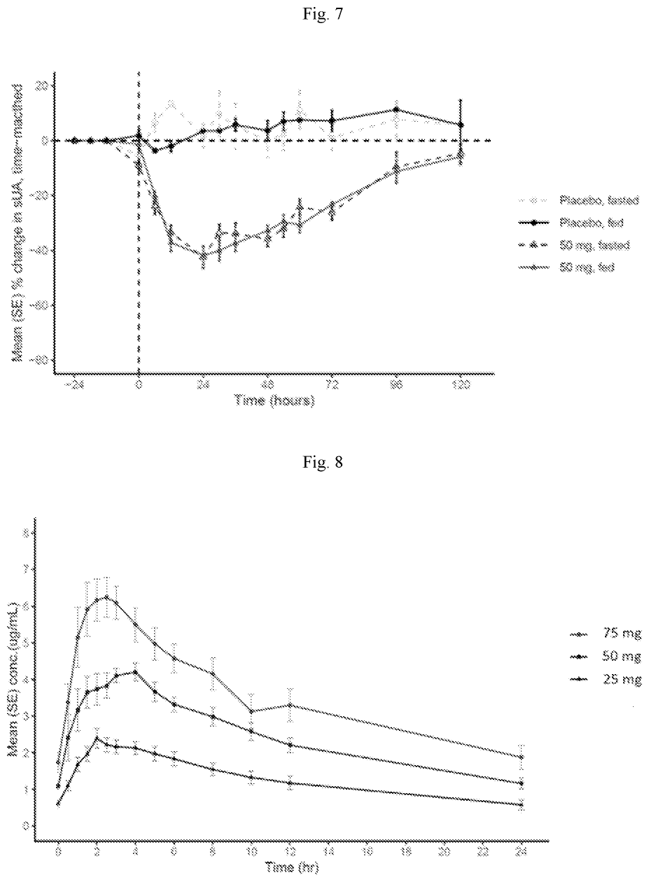

Despite a decrease in Cmax as a result of the fed state, there was no difference observed in sUA reduction throughout 120 hours post-dose when Compound 1 was administered (50 mg) with or without food (FIG. 7).

### Example 9: Phase 1 Clinical Trial with Compound 1 (Multiple Ascending Dose)

Compound 1 was tested in a Phase 1, randomized, double-blind, placebo-controlled study conducted to evaluate the safety, tolerability, pharmacokinetics (PK), and pharmacodynamics (PD) of multiple doses of Compound 1 in healthy adult males. A total of 24 subjects received multiple doses of Compound 1 (25 to 75 mg) in the fasted state. An additional 6 subjects received placebo.

### Pharmacokinetics

Following once-daily doses of Compound 1 at 25, 50 or 75 mg for 10 days, mild to moderate accumulation was observed. Accumulation were approximately 1.3 to 1.4 fold for C. and 1.4 to 1.5 fold for AUC_{0.24} (Table 12 and FIG. 8).

TABLE 12

| Dose | Food | | ^{a}Tₘₐₓ | Cₘₐₓ | AUC₀₋₂₄ | Rac | Rac |
|---|---|---|---|---|---|---|---|
| (mg) | N | | (hr) | (μg/mL) | (μg · h/mL) | Cₘₐₓ | AUC₀₋₂₄ |
| 25 | Fasted | Day 1 | 3.5 | 1.76 | 21.2 | — | — |
| | N = 8 | | (2.0-4.0) | (0.314) | (6.27) | | |
| | Fasted | Day 10 | 2.25 | 2.48 | 30.8 | 1.42 | 1.44 |
| | N = 8 | | (1.5-4.0) | (0.648) | (11.1) | (0.328) | (0.220) |
| 50 | Fasted | Day 1 | 2.0 | 3.55 | 39.6 | — | — |
| | N = 8 | | (1.0-4.0) | (0.625) | (6.48) | | |
| | Fasted | Day 10 | 2.5 | 4.59 | 58.2 | 1.30 | 1.47 |
| | N = 7 | | (1.0-5.0) | (0.653) | (12.5) | (0.158) | (0.104) |
| 75 | Fasted | Day 1 | 1.75 | 5.20 | 55.6 | — | — |
| | N = 8 | | (1.0-3.0) | (0.711) | (13.4) | | |
| | Fasted | Day 10 | 1.50 | 6.77 | 84.7 | 1.29 | 1.51 |
| | N = 8 | | (1.0-3.0) | (1.52) | (28.0) | (0.172) | (0.212) |

Abbreviations:
AUC₀₋₂₄, area under the concentration-time curve from 0 to 24 hours;
Cₘₐₓ, maximum concentration;
N, number of subjects;
Rac: accumulation ratio;
Tₘₐₓ, time to reach maximum concentration
^{a}Tₘₐₓ values are represented by median (range).

### Pharmacodynamics

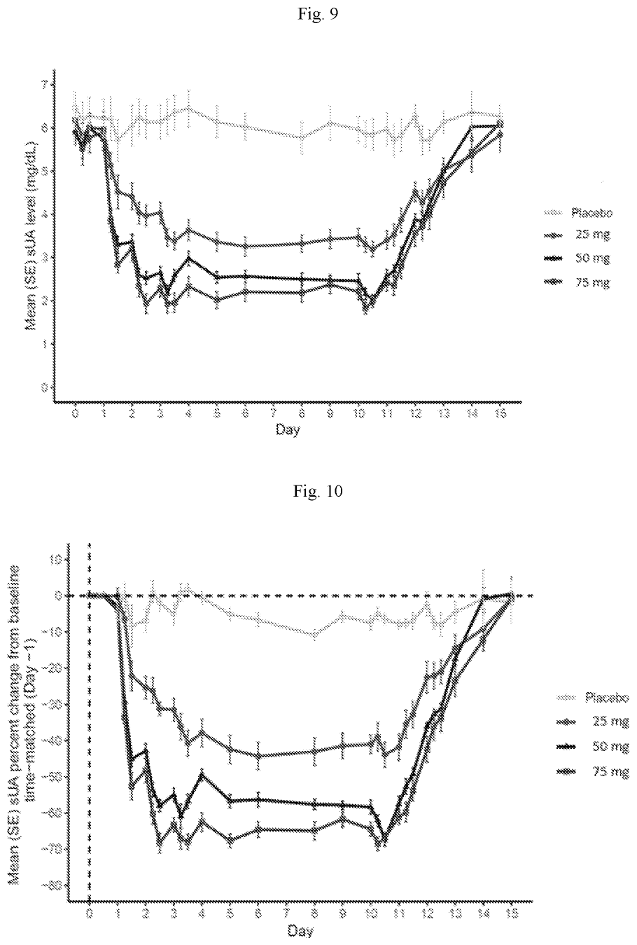

Upon administration of once-daily doses of Compound 1 at 25, 50 or 75 mg oral capsules under the fasted conditions for 10 days, the mean sUA levels showed dose-dependent decreases when compared to placebo group (FIG. 9). On Day 10, Compound 1 significantly reduced sUA concentration up to 44% (41% at trough) with 25 mg dose, up to 67% (58% at trough) with 50 mg and up to 69% (65% at trough) with 75 mg dose for 10 days (FIG. 10). In comparison, only 7.2% reduction was observed on Day 10 pre-dose in the placebo group. Steady-state sUA lowering effect was achieved after approximately 5 days.

### Example 10: Phase 2a Clinical Trial with Compound 1

In a phase 2a, single-center, two-sequence, cross-over study, adults with gout (sUA>7 mg/dL) were randomized to receive a once-daily, three-week treatment with Compound 1 50 mg alone, febuxostat 40 mg alone, or Compound 1 50 mg in combination with febuxostat 40 mg. An additional arm in the study included allopurinol 300 mg alone or Compound 1 50 mg in combination with allopurinol 300 mg. Serial blood samples were collected for measurement of sUA, Compound 1, febuxostat, or allopurinol pharmacokinetics and pharmacodynamics on end of treatment week (Day 7, Day 14 and Day 21). Urine samples were collected for assessment of uric acid excretion. Laboratory safety tests, vital signs, and electrocardiograms were collected throughout the study.

Following treatment of Compound 1, 96% of patients had sUA levels below 6 mg/dL, with 93% of patients below 5 mg/dL. Compound 1 reduced sUA from mean baseline (8.9 mg/dL) to 4.2 mg/dL at maximum on Day 7/Day 21, corresponding to 53% reduction. In comparison, febuxostat 40 mg reduced mean sUA levels to 5.5 mg/dL (38.9%) at maximum on Day 7/Day 21, with 67% and 33% patients below 6 and 5 mg/dL, respectively, and allopurinol 300 mg reduced mean sUA levels to 5.8 mg/dl maximally on Day 7/Day 21, with 56% and 11% patients below 6 and 5 mg/dL, respectively. The combination of Compound 1 and febuxostat further reduced mean sUA levels to 2.7 mg/dL (70.7% reduction) with fraction of patients<6, 5, 4 mg/dL as 100%, 100%, 88%, respectively. The combination of Compound 1 and allopurinol further reduced mean sUA levels to 3.0 mg/dL (66.0% reduction) with fraction of patients<6, 5, 4 mg/dL as 100%, 100%, 100%, respectively. Accounting for increased BMI and body weight of gout patients in the study, Compound 1 pharmacokinetics in gout patients was similar to healthy subjects. Compound 1 showed similar exposure between normal and mildly renal impaired patients. In mildly renal impaired patients, Compound 1 reduced sUA from 8.6±1.1 mg/dL mg/dL to 4.5±0.8 mg/dL. Compound 1 was well tolerated. There were no clinically significant laboratory or ECG abnormalities noted. Lastly, a subject in the study with albuminuria, showed a significant decrease in urine albumin-to-creatinine ratio (UACR) from 96 mg/g at baseline to 41 mg/g upon treatment with Compound 1 for 7 days (Table 13). Dosing with the Compound 1 and allopurinol combination for 7 days further reduced UACR to 35 mg/g (Day 14). However, after 7 additional days of only treating with allopurinol the subject's UACR increased to 55 mg/g (Day 21).

TABLE 13

| | | | Compound 1 50 mg/ | | |
|---|---|---|---|---|---|
| | | Compound 1 | ALLO 300 mg | | |
| Treatment | Baseline | 50 mg | Combination | ALLO 300 mg | Follow-up |
| Day | −1 | 7 | 14 | 21 | 28 |
| UACR(mg/g) | 96 | 41 | 35 | 55 | 83 |
| UACR % change | 0.0 | −57.4 | −63.9 | −42.6 | −13.0 |
| from baseline | | | | | |
| sUA % change | 0 | −50.9 | −61.8 | −40 | NA |
| from baseline | | | | | |

In summary, Compound 1 exhibited outstanding ability to reduce sUA level with high response rate at 50 mg in gout patients. sUA levels were reduced below 6 mg/dL at 24 hr post-dose in all patients following 7 days dosing of Compound 1.

### Example 11: Phase II Clinical Trial of Compound 1 or Compound 2 in Heart Failure with Preserved Election Fraction (HFpEF)

The primary objective of this Phase 2 study is to assess the effect of a combination of Compound 1 or Compound 2 and allopurinol on exercise capacity in patients with HFpEF.

Patients: Eligible subjects will be men and women 40 years of age and older.

Criteria:

Inclusion Criteria:
Capable of giving signed informed consent which includes compliance with the requirements and restrictions listed in the Informed Consent Form and in the protocol.
Provision of signed and dated, written Informed Consent Form prior to any mandatory study-specific procedures, sampling, and analyses.
Provision of signed and dated written genetic informed consent prior to collection of sample for genetic analysis.
Patient must be ≥40 years of age at the time of signing the Informed Consent Form.
Patients with hyperuricemia defined as serum uric acid level of >6 mg/dL.
Patients with documented diagnosis of symptomatic heart failure with preserved ventricular ejection fraction according to all of the following criteria:
Have New York Heart Association functional class II-III at enrolment
Have medical history of typical symptoms/signs of Heart Failure>6 weeks before enrolment, which is stably treated medically, with at least intermittent need for diuretic treatment. Typical symptoms of Heart Failure include breathlessness, orthopnoea, paroxysmal nocturnal dyspnoea, reduced exercise tolerance, fatigue, tiredness, increased time to recover after exercise, ankle swelling. Typical signs associated with Heart Failure include elevated jugular venous pressure, hepatojugular reflex, third heart sound (galop rhythm). Less specific symptoms include: weight gain (>2 kg/week), weight loss (in advanced Heart Failure), tissue wasting (cachexia), cardiac murmur, peripheral oedema (ankle, sacral, scrotal), pulmonary crepitations, reduced air entry and dullness to percussion at lung bases (pleural effusion), tachycardia, irregular pulse, tachypnoea, Cheyne-Stokes respiration, hepatomegaly, ascites, cold extremities, oliguria, narrow pulse pressure.
Left Ventricular Ejection Fraction≥45%
N-terminal pro b-type natriuretic peptide≥125 pg/mL (≥14.75 pmol/L) at Visit 2 for patients without ongoing atrial fibrillation/flutter. If ongoing atrial fibrillation/flutter at the time of sample collection, N-terminal pro b-type natriuretic peptide must be ≥250 pg/mL (≥29.51 pmol/L) or patients must have a history of pulmonary capillary wedge pressure≥15 mm Hg during rest or pulmonary capillary wedge pressure≥20 mm Hg during exercise.
Patients able to exercise to near exhaustion during a cardiopulmonary exercise test as exhibited by respiratory exchange ratio≥1.05 during cardiopulmonary exercise test conducted during screening. If patient does not achieve respiratory exchange ratio≥1.05 the cardiopulmonary exercise test may be repeated once, at least 48 hours but less than 2 weeks (but before randomization) after the initial test; in such cases the second test will serve as baseline.
Patients with peak volume of oxygen≤75% of expected using treadmill, or peak volume of oxygen≤68% of expected using cycle ergometer, based on normal values.
Patients treated according to locally recognized guidelines on standard-of-care treatment for patients with heart failure with preserved ventricular ejection fraction. Therapy should have been individually optimized and stable for ≥4 weeks (except diuretics) and include, unless contraindicated or not tolerated, treatment of high blood pressure (targeting a systolic blood pressure<130 mm Hg as suggested in 2017 American College of Cardiology/American Heart Association/Heart Failure Society of America Heart Failure guidelines), ischaemic heart disease, and atrial fibrillation.
Patients treated with a sodium-glucose transport protein 2 inhibitor or sacubitril/valsartan must be on stable dose for ≥4 weeks before randomisation.
Male or female
Negative pregnancy test (urine or serum) for female patients of childbearing potential. Female patients must be 1 year post-menopausal, surgically sterile, or using an acceptable method of contraception (with a failure rate of <1% per year) for the duration of the study (from the time they sign consent) and for 4 weeks after the last dose of study treatment to prevent pregnancy. Patients agreeing to total sexual abstinence can also be included, assuming it is their usual lifestyle.

Exclusion Criteria:
Estimated Glomerular Filtration Rate<30 ml/min/1.73 m² (based on Chronic Kidney Disease Epidemiology Collaboration formula)
Severe hepatic impairment (Child-Pugh class C)
Presence of any condition that precludes exercise testing such as:
Claudication that limits exertion
Uncontrolled bradyarrhythmia or tachyarrhythmia (according to Investigator judgement, pacemaker treatment is allowed as long as the same pacing mode/activity can be used at baseline and follow-up cardiopulmonary exercise test)
Clinically significant musculoskeletal disease or orthopaedic conditions that limit the ability to perform the cardiopulmonary exercise test (e.g., arthritis or injury in the foot, leg, knee or hip)
Severe obesity (body mass index≥50.0 kg/m2)
Amputation with artificial limb without stable prosthesis function for the past 3 months
Any condition that, in the opinion of the Investigator, would contraindicate cardiopulmonary exercise test assessment (e.g., severe visual impairment)
Any condition other than Heart Failure that, in the opinion of the Investigator, is the primary limitation to exercise
Known history of a documented left ventricular ejection fraction<40%
Probable alternative or concomitant diagnoses which in the opinion of the Investigator could account for the patient's Heart Failure symptoms and signs (e.g., anaemia, hypothyroidism)
Known carrier of the human leukocyte antigen HLA-B*58:01 allele. Patients of suspected Han Chinese, Korean or Thai descent, and patients from populations in whom the human leukocyte antigen HLA*B58:01 allele is known to be high shall perform genetical testing, for others the genetic test is at the discretion of the investigator
Patients diagnosed with tumor lysis syndrome or Lesch-Nyhan syndrome
Patients who are severely physically or mentally incapacitated and who in the opinion of investigator are unable to perform the patients' tasks associated with the protocol
Presence of any condition which, in the opinion of the investigator, places the patient at undue risk or potentially jeopardizes the quality of the data to be generated
Current acute decompensated Heart Failure or hospitalization due to decompensated Heart Failure<4 weeks prior to enrolment
Myocardial infarction, unstable angina, coronary revascularization (percutaneous coronary intervention or coronary artery bypass grafting), ablation of atrial flutter/fibrillation, valve repair/replacement, implantation of a cardiac resynchronization therapy device, stroke or transient ischemic attack within 6 months prior to enrolment
Planned coronary revascularization, ablation of atrial flutter/fibrillation and/or valve repair/replacement
Atrial fibrillation with persistent resting heart rate>110 beats per minute.
Primary pulmonary hypertension, chronic pulmonary embolism, severe pulmonary disease including chronic obstructive pulmonary disease (COPD) (i.e., requiring home supplemental oxygen, chronic oral steroid therapy use at a dose equivalent to 10 mg prednisone or greater, or hospitalization for exacerbation of chronic obstructive pulmonary disease COPD requiring ventilatory assist within 12 months prior to enrolment)
Previous cardiac transplantation, or complex congenital heart disease. Planned cardiac resynchronization therapy. Prior implantation of a ventricular assistance device or similar device, or implantation expected during the course of the study
Heart Failure due to known infiltrative cardiomyopathy (e.g., amyloid, sarcoid, lymphoma, endomyocardial fibrosis, hemochromatosis), active myocarditis, constrictive pericarditis, cardiac tamponade, known genetic hypertrophic cardiomyopathy or obstructive hypertrophic cardiomyopathy, arrhythmogenic right ventricular cardiomyopathy/dysplasia, or uncorrected severe primary valvular disease
QT interval corrected by the Fridericia formula (QTcF)>470 msec; in patients with QRS interval (<120 ms), patients diagnosed with long QT syndrome; patients with a family history of long QT syndrome
Uncontrolled hypertension with systolic blood pressure>160 mm Hg and/or diastolic blood pressure>100 mm Hg
History of blood dyscrasias: Myelosuppression (e.g., thrombocytopenia, leukopenia, granulocytopenia, pancytopenia) and aplastic anaemia
Patients with the following bilateral upper or lower arm pathology can be randomized into the study, but are not allowed to participate in the reactive hyperaemia assessment:
Presence of fistula/arteriovenous (AV) Shunt
Other structural or vascular abnormality
Treated with any drug for hyperuricaemia in the 6 months preceding randomisation. Drugs for hyperuricaemia include all xanthine oxidase inhibitors (allopurinol, febuxostat and topiroxostat) and uric acid transporter 1 inhibitors (lesinurad, verinurad, probenecid, and benzbromarone) and urate oxidases (pegloticase, rasburicase).
Treated with strong or moderate organic anion transporting polypeptide (OATP)
inhibitors (Entresto is not considered a strong or moderate organic anion transporting polypeptide (OATP) inhibitor).
Patients treated with strong P-glycoprotein and/or CYP3A4 inhibitors due to the potential drug-drug interaction with colchicine
Participation in another clinical study with an investigational product administered (currently or within 1 month prior to screening).
Participating in a structured exercise training program in the 1 month prior to screening or planned to start during the trial
Claustrophobia
Involvement in the planning and/or conduct of the study (applies to both AZ staff and/or staff at the study site).
Judgment by the investigator that the patient should not participate in the study if the patient is unlikely to comply with study procedures, restrictions and requirements.
Screened more than once or previous randomisation in the present study.
Known hypersensitivity to or previous anaphylactic reaction to allopurinol or any organic anion transporting polypeptide inhibitor, including severe cutaneous adverse reaction triggered by allopurinol.
Patients who are pregnant (confirmed with pregnancy test), lactating, or planning to become pregnant.

Study Design:
Allocation: Randomized
Intervention Model: Parallel Assignment
Masking: Triple Blind (Participant, Care Provider, Investigator)
Primary Purpose: Treatment

Primary Outcome Measures:
Peak V02 Change from baseline at Week 28 in exercise capacity (Compound 1 or 2+allopurinol vs. placebo) [Time Frame: From baseline at Week 28] to assess effect of Compound 1 or 2+allopurinol compared to placebo on exercise capacity

Secondary Outcome Measures:
Peak V02 Change from baseline at Week 28 in exercise capacity (Compound 1 or 2+allopurinol vs. placebo+allopurinol) [Time Frame: From baseline at Week 28] to assess effect of Compound 1 or 2+allopurinol compared to allopurinol monotherapy on exercise capacity
Change from baseline at Week 28 in Kansas-City Cardiomyopathy Questionnaire-Total Symptom Score (KCCQ-TSS) [Time Frame: From baseline at Week 28] to assess effect of Compound 1 or 2+allopurinol compared to placebo on Kansas-City Cardiomyopathy Questionnaire-Total Symptom Score (KCCQ-TSS). The score ranges from 0 to 100, where a higher score represents a better patient outcome
Change from baseline at Week 28 in Kansas-City Cardiomyopathy Questionnaire-Total Symptom Score (KCCQ-TSS) [Time Frame: From baseline at Week 28] to assess effect of Compound 1 or 2+allopurinol compared to allopurinol monotherapy on Kansas-City Cardiomyopathy Questionnaire-Total Symptom Score (KCCQ-TSS). The score ranges from 0 to 100, where a higher score represents a better patient outcome.

| Arms | Assigned Interventions |
|---|---|
| Experimental: Compound 1 or 2 + | Drug: Compound 1 or 2 |
| allopurinol | The treatment will be titrated in 3 steps for target low dose |
| Dose [mg] Compound 1 or 2/allopurinol: | (25 mg), intermediate dose (50 mg) and high dose (75 mg) of |
| Step 1 - titration 25/100 Step 2 - titration | Compound 1 or 2. |
| 50/200 Step 2 - target dose 75/300 | Drug: Allopurinol The treatment will be titrated in 3 steps. |
| | Low dose (100 mg), intermediate (200 mg) and high dose |
| | (300 mg) of allopurinol. |
| | Other Names: |
| | Compound 1 or 2 titration 25 - 50 - 75 mg |
| | allopurinol titration 100 - 200 - 300 mg |
| Experimental: Allopurinol alone | Drug: Allopurinol |
| Dose [mg] Compound 1 or 2 /allopurinol: | Study treatments will be titrated in 3 steps: Low dose (100 |
| Step 1 - titration 0/100 Step 2 - titration | mg), intermediate (200 mg) and high dose (300 mg) of |
| 0/200 Step 3 - target dose 0/300 | allopurinol |
| | Other Name: allopurinol titration 100 - 200 - 300 mg |
| Placebo Comparator: Placebo | Drug: Placebo for Compound 1 or 2 |
| Placebo [mg] in 3 steps 0/0 | Matching Capsule |
| | Other Name: Placebo |
| | Drug: Placebo for allopurinol |
| | Matching tablet |
| | Other Name: Placebo |

The examples and embodiments described herein are for illustrative purposes only and in some embodiments, various modifications or changes are to be included within the purview of disclosure and scope of the appended claims.

We claim:

1. A method for treating or preventing chronic kidney disease in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone.

2. The method of claim 1 for treating chronic kidney disease in an individual in need thereof.

3. The method of claim 1 for preventing chronic kidney disease in an individual in need thereof.

4. A method for treating or preventing heart failure in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone.

5. The method of claim 4 for treating heart failure in an individual in need thereof.

6. The method of claim 4 for preventing heart failure in an individual in need thereof.

7. A method for treating hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, in an individual in need thereof, in an individual in need thereof, comprising administering to the individual a therapeutically effective amount of (3,5-dibromo-4-hydroxyphenyl)(2-(1-hydroxyethyl)benzofuran-3-yl-4,5,6,7-d₄)methanone.

8. The method of any one of claims 1-7, wherein the therapeutically effective amount is from about 3 mg to about 1500 mg.

9. The method of any one of claims 1-8, wherein the therapeutically effective amount is from about 3 mg to about 600 mg.

10. The method of any one of claims 1-9, wherein the therapeutically effective amount is from about 5 mg to about 300 mg.

11. The method of any one of claims 1-10, wherein the therapeutically effective amount is from about 10 mg to about 200 mg.

12. The method of any one of claims 1-11, wherein the therapeutically effective amount is from about 10 mg to about 100 mg.

13. The method of any one of claims 1-12, wherein (3,5-dibromo-4-hydroxyphenyl)(2-(1-hydroxyethyl)benzofuran-3-yl-4,5,6,7-d₄)methanone is administered orally.

14. The method of claim 13, wherein the therapeutically effective amount is taken with food.

15. The method of claim 13, wherein the therapeutically effective amount is taken without food.

16. The method of any one of claims 1-15, wherein the therapeutically effective amount is administered to the individual once per day.

17. The method of any one of claims 1-15, wherein the therapeutically effective amount is administered to the individual twice per day.

18. The method of any one of claims 1-17, further comprising administering at least one additional therapeutic agent.

19. The method of any one of claims 1-18, further comprising administering a xanthine oxidase inhibitor.

20. The method of claim 19, wherein the xanthine oxidase inhibitor is allopurinol, oxypurinol, febuxostat, topiroxostat, or inositol.

21. The method of any one of claims 1-20, further comprising administering an SGLT2 inhibitor.

22. The method of claim 21, wherein the SGLT2 inhibitor is canagliflozin, dapagliflozin, empagliflozin, empagliflozin/linagliptin, empagliflozin/metformin, or dapagliflozin/metformin.

23. A pharmaceutical composition for use in the treatment or prevention of chronic kidney disease, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone, and at least one inactive ingredient selected from pharmaceutically acceptable carriers, diluents, and excipients.

24. The pharmaceutical composition of claim 23 for use in the treatment of chronic kidney disease in an individual in need thereof.

25. The pharmaceutical composition of claim 23 for use in the prevention of chronic kidney disease in an individual in need thereof.

26. A pharmaceutical composition for use in the treatment or prevention of heart failure, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone, and at least one inactive ingredient selected from pharmaceutically acceptable carriers, diluents, and excipients.

27. The pharmaceutical composition of claim 26 for use in the treatment of heart failure in an individual in need thereof.

28. The pharmaceutical composition of claim 26 for use in the prevention of heart failure in an individual in need thereof.

29. A pharmaceutical composition for use in the treatment of hypertension, coronary artery disease, Lesch-Nyhan syndrome, Kelley-Seegmiller syndrome, kidney stones, kidney failure, diabetic kidney disease, joint inflammation, arthritis, urolithiasis, plumbism, hyperparathyroidism, psoriasis, sarcoidosis, hypoxanthine-guanine phosphoribosyltransferase (HPRT) deficiency, or a combination thereof, wherein the pharmaceutical composition comprises a therapeutically effective amount of (3,5-dibromo-4-hydroxyphenyl)(2-(1-hydroxyethyl)benzofuran-3-yl-4,5,6,7-d₄)methanone, and at least one inactive ingredient selected from pharmaceutically acceptable carriers, diluents, and excipients.

30. The pharmaceutical composition of any one of claims 23-29, wherein the therapeutically effective amount is from about 3 mg to about 1500 mg.

31. The pharmaceutical composition of any one of claims 23-30, wherein the therapeutically effective amount is from about 3 mg to about 600 mg.

32. The pharmaceutical composition of any one of claims 23-31, wherein the therapeutically effective amount is from about 5 mg to about 300 mg.

33. The pharmaceutical composition of any one of claims 23-32, wherein the therapeutically effective amount is from about 10 mg to about 200 mg.

34. The pharmaceutical composition of any one of claims 23-33, wherein the therapeutically effective amount is from about 10 mg to about 100 mg.

35. The pharmaceutical composition of any one of claims 23-34, wherein the therapeutically effective amount is from about 25 mg to about 75 mg.

36. The pharmaceutical composition of any one of claims 23-35 formulated for oral, intravenous, intramuscular, or subcutaneous administration.

37. The pharmaceutical composition of any one of claims 23-36 formulated for oral administration.

38. The pharmaceutical composition of claim 37, wherein the therapeutically effective amount is taken with food.

39. The pharmaceutical composition of claim 37, wherein the therapeutically effective amount is taken without food.

40. The pharmaceutical composition of any one of claims 23-39, wherein the therapeutically effective amount is administered once per day.

41. The pharmaceutical composition of any one of claims 23-39, wherein the therapeutically effective amount is administered twice per day.

42. The pharmaceutical composition of any one of claims 23-41, further comprising at least one additional therapeutic agent.

43. The pharmaceutical composition of any one of claims 23-42, further comprising a xanthine oxidase inhibitor.

44. The pharmaceutical composition of claim 43, wherein the xanthine oxidase inhibitor is allopurinol, oxypurinol, febuxostat, topiroxostat, or inositol.

45. The pharmaceutical composition of any one of claims 23-44, further comprising administering an SGLT2 inhibitor.

46. The pharmaceutical composition of claim 45, wherein the SGLT2 inhibitor is canagliflozin, dapagliflozin, empagliflozin, empagliflozin/linagliptin, empagliflozin/metformin, or dapagliflozin/metformin.
